(19) United States
(12) Patent Application Publication
(10) Pub. No.: US 2026/0048049 A1
(43) Pub. Date: Feb. 19, 2026

(54) METHODS FOR TREATING CANCER USING INHALED ANGIOGENESIS INHIBITOR

(71) Applicant: Pulmatrix Operating Company, Inc., Framingham, MA (US)

(72) Inventor: Aidan K. CURRAN, Duxbury, MA (US)

(21) Appl. No.: 19/104,475

(22) PCT Filed: Aug. 17, 2023
(86) PCT No.: PCT/US2023/072395

§ 371 Date: Feb. 18, 2025

Related U.S. Application Data
(60) Provisional application No. 63/399,080, filed on Aug. 18, 2022.

(51) Int. Cl. A61K 31/496; A61K 9/00; A61K 9/16; A61K 45/06

(52) U.S. Cl. CPC A61K 31/496; A61K 9/0075; A61K 9/1611; A61K 9/1617; A61K 9/1623; A61K 45/06

(57) ABSTRACT

The present disclosure relates to methods of treating cancer, such as lung cancer (e.g., non-small cell lung cancer) by administering a dry powder to the respiratory tract of a subject in need thereof (e.g., by oral inhalation), the dry powder comprising respirable dry particles that comprise an angiogenesis inhibitor (e.g., itraconazole), and optionally a stabilizer (e.g., polysorbate 80) and/or one or more excipients (e.g., leucine and a sodium salt, such as sodium sulfate).

### RELATED APPLICATIONS

This application claims the benefit of U.S. Provisional Application No. 63/399,080, filed on Aug. 18, 2022, which is incorporated herein by reference in its entirety.

### BACKGROUND

Lung cancer is the third most common form of cancer, and the leading cause of cancer deaths in the United States. The five-year survival rate for lung cancer is only 56 percent when it is detected while still localized, and only 16 percent of lung cancer cases are diagnosed at this early stage. Once the lung cancer spreads to other organs the five-year survival rate drops to only 5 percent. As such, more than half of lung cancer patients will die within one year of being diagnosed. (See e.g., U.S. National Institute Of Health, National Cancer Institute. SEER Cancer Statistics Review, 1975-2015). Thus, new and effective lung cancer treatments are urgently needed.

Because non-small cell lung cancer (NSCLC) is a highly vascularized tumor, angiogenesis inhibitors can be used to treat subjects with NSCLC, either alone or in addition to another cancer treatment (e.g., as a neoadjuvant). However, there are only three angiogenesis inhibitors approved by the FDA for use in NSCLC, and those drugs provide only modest overall survival benefits, in addition to potentially causing serious side-effects and mortality rates, and are expensive. (See, e.g., Aftab et al. Cancer Res. (2011) 71:6764-6772: Daum et al. Front. Cell Dev. Biol. (2021) 9:1-17).

Itraconazole was recently identified as an angiogenesis inhibitor and an antagonist of the hedgehog signaling pathway, and it has been studied in the treatment of NSCLC. Specifically, clinical trial subjects with NSCLC were administered large amounts of oral itraconazole, and it was found that tissue concentrations of itraconazole were significantly associated with reduction in tumor volume and tumor perfusion, decrease in the proangiogenic cytokines IL1b and GM-CSF, and reduction in tumor microvessel density (Gerber et al. Clin. Cancer Res. (2020) 26:6017-6027).

Itraconazole is a well-known small-molecule drug that has been available for over three decades and is typically used for treating fungal infections, and is the active ingredient in the antifungal drug SPORANOX® (itraconazole; Janssen Pharmaceuticals). Itraconazole can be synthesized using a variety of methods that are well known in the art. Therefore, itraconazole appears to be a suitable alternative to angiogenesis inhibitors currently approved for treating NSCLC. However, itraconazole has a low aqueous solubility and poor oral bioavailability, with unpredictable and heterogenous pharmacokinetic parameters, and extensive drug-drug interactions. Consequently, itraconazole commonly leads to side effects and toxicity in patients, and obtaining pharmaceutical formulations that provide safe and therapeutic levels of itraconazole has been challenging. For example, in the clinical study of high dose oral itraconazole in NSCLC patients, pharmacokinetic parameters varied more than 6-fold across the patient population, and only two subjects achieved the expected plasma concentrations of itraconazole (Gerber, supra). Considering the significant association between itraconazole tissue concentration and tumor volume reduction, the unpredictable pharmacokinetics of oral itraconazole is a significant obstacle for its application in the treatment of cancer, and the poor pharmacokinetics and significant side effects of oral itraconazole further limits its use in therapy.

A need exists for new methods of treating cancer, particularly lung cancer, using a formulation of an angiogenesis inhibitor that can achieve therapeutically effective concentrations in the cancer tissue consistently, while avoiding high plasma concentrations and toxicity.

### SUMMARY OF THE INVENTION

The present disclosure relates to methods of treating cancer, such as lung cancer, e.g., non-small cell lung cancer (NSCLC), by administering a respirable dry powder to a subject in need thereof by inhalation (e.g., oral inhalation), wherein the dry powder comprises homogenous respirable dry particles that comprise an angiogenesis inhibitor (e.g., itraconazole), and optionally a stabilizer (e.g., polysorbate 80) and/or one or more excipients (e.g., a sodium salt and leucine).

A cancer treated by a method disclosed herein may be a lung cancer, such as NSCLC. The cancer (e.g., lung cancer) may be a locally advanced cancer. In some embodiments, the cancer (e.g., lung cancer) is a metastatic cancer (or stage IV cancer). The cancer (e.g., lung cancer) may be refractory, recurrent, or both.

A method disclosed herein may comprise administering to a subject the dry powder with an additional therapeutic agent. The additional therapeutic agent may be a therapeutic agent disclosed herein, such as a chemotherapeutic drug, a targeted cancer therapy drug, an immunotherapy drug, or a combination thereof. In some embodiments, the additional therapeutic agent is a chemotherapeutic drug. In some embodiments, the additional therapeutic agent is an immunotherapy drug. In some embodiments, the additional therapeutic agent is a targeted cancer therapy drug.

In some aspects, a method of treating cancer (e.g., lung cancer) disclosed herein comprises: (i) administering to the respiratory tract of a subject in need thereof a dry powder, wherein the dry powder comprises homogenous respirable dry particles that comprise an angiogenesis inhibitor (e.g., itraconazole), and optionally a stabilizer (e.g., polysorbate 80) and/or one or more excipients (e.g., leucine and a sodium salt); and (ii) administering an additional therapeutic agent, e.g., a chemotherapeutic drug, a targeted cancer therapy drug, an immunotherapy drug, or a combination thereof, to the subject.

In some aspects, a method of treating cancer (e.g., lung cancer) disclosed herein comprises: (i) administering to the respiratory tract of a subject in need thereof a dry powder, wherein the dry powder comprises homogenous respirable dry particles that comprise an angiogenesis inhibitor (e.g., itraconazole), and optionally a stabilizer (e.g., polysorbate 80) and/or one or more excipients (e.g., leucine and a sodium salt); (ii) orally administering itraconazole to the subject; and (iii) optionally, administering an additional therapeutic agent (e.g., a chemotherapeutic drug, a targeted cancer therapy drug, an immunotherapy drug, or a combination thereof) to the subject.

In a method disclosed herein comprising a step of administering an additional therapeutic agent and/or a step of orally administering itraconazole, in addition to the dry powder, the dry powder may be administered at any time and in any order in relation to the additional therapeutic agent and/or oral itraconazole. For example, a dose of the additional therapeutic agent and/or oral itraconazole may be administered no more than 24 hours before or after administering a dose of the dry powder, e.g., within about 12 hours, within about 6 hours, within about 4 hours, within about 3 hours, within about 2 hours, within about 1 hour, or within about 30 minutes, or less, of the dry powder. Alternatively, the dry powder may be administered to the subject more than a day before or a day after administering the additional chemotherapeutic and/or oral itraconazole, e.g., 2 days before or after, 3 days before or after. 4 days before or after, 5 days before or after, 6 days before or after, 1 week before or after, 2 weeks before or after, 3 weeks before or after, 4 weeks before or after, or longer. In some embodiments, the dry powder is administered to the subject between about 1 and about 28 days before administering an additional chemotherapeutic and/or oral itraconazole, e.g., 1 to about 21 days, about 1 to about 18 days, about 1 to about 16 days, about 1 to about 14 days, about 1 to about 12 days, or about 1 to about 10 days, before administering the additional chemotherapeutic and/or oral itraconazole.

In some embodiments, the additional therapeutic agent is used for treating cancer, such as a platinum-based drug, an antimetabolite, an antimicrotubule agent, a topoisomerase inhibitor, an anthracycline, a KRAS (Kirsten rat sarcoma viral oncogene homolog) inhibitor, an ALK (anaplastic lymphoma kinase) inhibitor, an EGFR (epidermal growth factor receptor) inhibitor, a VEGF (vascular endothelial growth factor) inhibitor, a BRAF inhibitor, a MEK inhibitor, a RET inhibitor, a MET inhibitor, or an immunotherapy drug (e.g., an immune checkpoint inhibitor, e.g., a PD-1/PD-L1 inhibitor or CTLA-4 inhibitor). In some embodiments, the additional therapeutic agent is selected from the group consisting of pemetrexed, cisplatin, carboplatin, oxaliplatin, satraplatin, picoplatin, nedaplatin, triplatin, lipoplatin, paclitaxel, docetaxel, doxorubicin, gemcitabine, vinorelbine, etoposide, SN-38, camptothecin, topotecan, exatecan, irinotecan, belotecan, methotrexate, bevacizumab, ranibizumab, aflibercept, ramucirumab, nintedanib, erlotinib, afatinib, axitinib, gefitinib, cabozantinib, osimertinib, dacomitinib, sotorasib, crizotinib, entrectinib, lenvatinib, pazopanib, ceritinib, alectinib, brigatinib, lorlatinib, dabrafenib, regorafenib, sorafenib, vemurafenib, sunitinib, everolimus, thalidomide, lenalidomide, trametinib, vandetanib, selpercatinib, pralsetinib, capmatinib, tepotinib, larotrectinib, amivantamab, mobocertinib, nivolumab, ipilimumab, atezolizumab, pembrolizumab, tremelimumab, cetuximab, cemiplimab, pidilizumab, durvalumab, necitumumab, and combinations thereof.

In some embodiments, the dry powder is administered to the subject before, concurrently with, or after performing surgery, such as a surgery to remove cancerous tissue from the subject (e.g., a segmentectomy, a sleeve resection, a wedge resection, a lobectomy, a pneumonectomy, a lymphadenectomy, or a combination thereof).

In some embodiments, the dry powder is administered to the subject before, concurrently with, or after performing radiation therapy to treat the cancer (e.g., brachytherapy, external beam radiation therapy (EBRT), stereotactic body radiation therapy (SBRT), stereotactic ablative radiotherapy (SABR), three-dimensional conformal radiation therapy (3D-CRT), intensity modulated radiation therapy (IMRT), volumetric modulated arc therapy (VMAT), stereotactic radiosurgery (SRS), radiofrequency ablation, or a combination thereof).

In some embodiments, the angiogenesis inhibitor of the dry powder administered to the subject is at least 50% crystalline, e.g., 60% crystalline, 70% crystalline, 80% crystalline, 90% crystalline, 92% crystalline, 94% crystalline, 95% crystalline, 96% crystalline, 97% crystalline, 98% crystalline, 99% crystalline, 99.5% crystalline, 99.9% crystalline, or more. In some embodiments, the angiogenesis inhibitor is itraconazole. The itraconazole may be crystalline itraconazole. In some embodiments, the itraconazole is not amorphous itraconazole.

The dry powder used in a method disclosed herein may comprise an angiogenesis inhibitor in sub-particle form, wherein the sub-particle is about 50 nm to about 5,000 nm (Dv50), e.g., about 50 nm to about 800 nm (Dv50), about 50 nm to about 300 nm (Dv50), about 50 nm to about 200 nm (Dv50), or about 100 nm to about 300 nm (Dv50). In some embodiments, the angiogenesis inhibitor is a crystalline sub-particle.

The angiogenesis inhibitor may be present in the respirable dry particles in an amount of about 1% to about 95% by weight, e.g., about 40% to about 90% by weight, about 55% to about 85% by weight, about 55% to about 75% by weight, about 65% to about 85% by weight, or about 40% to about 60% by weight.

In some embodiments, the dry powder comprises a stabilizer (e.g., polysorbate 80) and one or more excipients (e.g., sodium sulfate and leucine). The ratio of angiogenesis inhibitor:stabilizer (wt:wt) in the respirable dry particles may be in any desired ratio, for example, from about 1:1 to 50:1; greater than or equal to 10:1; about 10:1; about 20:1; about 5:1 to about 20:1; about 7:1 to about 15:1; or about 9:1 to about 11:1. In some embodiments, the stabilizer is present in the respirable dry particles in an amount of about 0.05% to about 45% by weight, e.g., about 4% to about 10% by weight. The one or more excipients may be present in the respirable dry particles in an amount of about 10% to about 99% by weight. For example, the one or more excipients may be present in the respirable dry particles in an amount of about 5% to about 50% by weight.

The one or more excipients present in the dry powder may comprise a monovalent metal cation salt, a divalent metal cation salt, an amino acid, a sugar alcohol, or combinations thereof. In some embodiments, the one or more excipients comprise a sodium salt and an amino acid. The sodium salt may be selected from the group consisting of sodium chloride and sodium sulfate. The amino acid may be leucine.

In some embodiments, the stabilizer present in the respirable dry particles is polysorbate 80, in an amount of 10 wt % or less, e.g., 7 wt % or less, 5 wt % or less, 4 wt % or less, 3 wt % or less, 2 wt % or less, or 1 wt % or less.

In some embodiments, the stabilizer present in the respirable dry particles is oleic acid or a salt thereof, in an amount of 10 wt % or less, e.g., 7 wt % or less, 5 wt % or less, 4 wt % or less, 3 wt % or less, 2 wt % or less, or 1 wt % or less.

In some embodiments, the respirable dry particles have: (i) a volume median geometric diameter (VMGD) of about 10 microns or less, e.g., about 5 microns or less; (ii) a tap density of about 0.2 g/cc or greater, e.g., a tap density of between 0.2 g/cc and 1.0 g/cc; (iii) a 1 bar/4 bar dispersibility ratio (1/4 bar) of less than about 1.5, as measured by laser diffraction; and/or (iv) a 0.5 bar/4 bar dispersibility ratio (0.5/4 bar) of about 1.5 or less, as measured by laser diffraction.

In some embodiments, the dry powder has: (i) a mass median aerodynamic diameter (MMAD) of between about 1 micron and about 5 microns; and/or (ii) a fine particle fraction (FPF) of the total dose less than 5 microns of about 25% or more.

In some embodiments, the respirable dry particles have a capsule emitted powder mass of at least 80% when emitted from a passive dry powder inhaler that has a resistance of about 0.036 sqrt (kPa)/liters per minute under the following conditions: an inhalation flow rate of 30 LPM for a period of 3 seconds using a size 3 capsule that contains a total mass of 10 mg, said total mass consisting of the respirable dry particles, and wherein the volume median geometric diameter of the respirable dry particles emitted from the inhaler as measured by laser diffraction is 5 microns or less.

A method disclosed herein may comprise administering the dry powder to the respiratory tract of a subject using a passive dry powder inhaler, such as a capsule-based passive dry powder inhaler.

In some embodiments, the dry powder is administered in an amount effective to achieve a lung concentration in sputum (e.g., steady state concentration in sputum) of the angiogenesis inhibitor (e.g., itraconazole) of at least 100 ng/ml, such as about 500 ng/ml, about 800 ng/mL, about 1200 ng/mL, about 1600 ng/mL, about 2000 ng/mL, about 3000 ng/mL, about 4000 ng/mL, about 5000 ng/mL, about 6000 ng/mL, about 7000 ng/mL, about 8000 ng/mL, about 10,000 ng/mL, about 15,000 ng/mL, about 20,000 ng/mL, about 25,000 ng/mL, about 50,000 ng/mL, about 75,000 ng/mL, about 100,000 ng/mL, about 125,000 ng/mL, about 150,000 ng/mL, about 175,000 ng/mL, about 200,000 ng/mL, or more; or between about 500 ng/mL to 400,000 ng/mL, between about 500 ng/mL to 300,000 ng/mL, between about 500 ng/mL to 200,000 ng/mL, between about 500 ng/mL to 100,000 ng/mL, between about 500 ng/mL to 50,000 ng/mL, between about 500 ng/mL to 25,000 ng/mL, between about 500 ng/mL to 10,000 ng/mL, about 2000 ng/mL to 8000 ng/mL, or about 2000 ng/mL to 8100 ng/mL.

In some embodiments, the dry powder is administered in an amount effective to achieve a lung tissue concentration (e.g., steady state concentration in the tissue), e.g., in lung tumor tissue, of the angiogenesis inhibitor (e.g., itraconazole) of at least about 100 ng/g, such as about 500 ng/g, about 800 ng/g, about 1200 ng/g, about 1600 ng/g, about 2000 ng/g, about 3000 ng/g, about 4000 ng/g, about 5000 ng/g, about 6000 ng/g, about 7000 ng/g, about 8000 ng/g, about 9000 ng/g, about 10,000 ng/g, or more, e.g., up to about 1.6 mg/g, up to about 1.4 mg/g, up to about 1.2 mg/g, or up to about 1.0 mg/g; or between about 100 ng/g and about 900,000 ng/g, e.g., between about 100 ng/g and about 800,000 ng/g, between about 100 ng/g and about 700,000 ng/g, between about 100 ng/g and about 600,000 ng/g, between about 100 ng/g and about 500,000 ng/g, between about 100 ng/g and about 400,000 ng/g, between about 100 ng/g and about 300,000 ng/g, between about 100 ng/g and about 200,000 ng/g, or between about 100 ng/g and about 100,000 ng/g; or between about 100 ng/g and about 1.6 mg/g. e.g., about 500 ng/g, about 1000 ng/g, about 1500 ng/g, about 2000 ng/g, about 2500 ng/g, about 5000 ng/g, about 10,000 ng/g, about 15,000 ng/g, about 20,000 ng/g, about 25,000 ng/g, about 30,000 ng/g, about 40,000 ng/g, about 50,000 ng/g, about 60,000 ng/g, about 70,000 ng/g, about 80,000 ng/g, about 90,000 ng/g, about 100,000 ng/g, about 120,000 ng/g, about 140,000 ng/g, about 160,000 ng/g, about 180,000 ng/g, about 200,000 ng/g, about 250,000 ng/g, about 300,000 ng/g, about 350,000 ng/g, about 400,000 ng/g, about 450,000 ng/g, about 500,000 ng/g, about 600,000 ng/g, about 700,000 ng/g, about 800,000 ng/g, about 900,000 ng/g, about 1 mg/g, about 1.2 mg/g, about 1.4 mg/g, or about 1.6 mg/g.

In some embodiments, the dry powder is administered in an amount effective to achieve a plasma concentration (e.g., steady state concentration) of the angiogenesis inhibitor (e.g., itraconazole) of no more than 25 ng/mL.

In some embodiments, the dry powder is administered to the subject as a single dose (e.g., a single dose administered via oral inhalation). In some embodiments, the dry powder is administered as an initial dose followed by one or more subsequent doses.

In other aspects, the present disclosure relates to a dry powder (e.g., a dry powder disclosed herein) for use in treating cancer (e.g., lung cancer, e.g., NSCLC), wherein the dry powder comprises homogenous respirable dry particles that comprise an angiogenesis inhibitor (e.g., itraconazole), and optionally a stabilizer (e.g., polysorbate 80) and/or one or more excipients (e.g., a sodium salt and leucine), and wherein the dry powder is for administration to the respiratory tract of a subject.

In other aspects, the present disclosure relates to use of a dry powder (e.g., a dry powder disclosed herein) in the manufacture of a medicament for the treatment of cancer, (e.g., lung cancer, e.g., NSCLC), wherein the dry powder comprises homogenous respirable dry particles that comprise an angiogenesis inhibitor (e.g., itraconazole), and optionally a stabilizer (e.g., polysorbate 80) and/or one or more excipients (e.g., a sodium salt and leucine), and wherein the medicament is for administration to the respiratory tract of a subject.

### BRIEF DESCRIPTION OF THE DRAWINGS

FIG. 1 is a particle X-ray diffraction plot for Formulations I and II.

FIG. 2 is a particle X-ray diffraction plot for Formulations III and IV.

FIG. 3 is a particle X-ray diffraction plot for Formulations V and VI.

FIG. 4 is a particle X-ray diffraction plot for Formulations VII and VIII.

FIG. 5 is a particle X-ray diffraction plot for Formulation XI.

FIG. 6 is a particle X-ray diffraction plot for Formulation XII.

FIG. 7 is a particle X-ray diffraction plot for Formulation XIII.

FIG. 8 is a particle X-ray diffraction plot for Formulation XIV.

FIG. 9 is a particle X-ray diffraction plot for Formulation XV.

FIG. 10 is a particle X-ray diffraction plot for Formulation XVI.

FIG. 11 is a particle X-ray diffraction plot for Formulation XIX.

FIG. 12 is a plot providing cumulative mass dissolution of the impactor stage mass (ISM) collected post-aerosolization of different dry powders comprising the angiogenesis inhibitor itraconazole from an RS01 dry powder inhaler (DPI) at 60 L/min in the UniDose and then paddle over disk (POD) dissolution testing in a USP Apparatus II set-up.

FIG. 13 is a plot providing cumulative percentage mass dissolution of the ISM collected post-aerosolization of different dry powders comprising the angiogenesis inhibitor itraconazole from an RS01 DPI at 60 L/min in the UniDose and then POD dissolution testing in a USP Apparatus II set-up.

FIG. 14A is a plot illustrating the relationship between the dissolution half-life and particle size of the angiogenesis inhibitor itraconazole in different dry powders.

FIG. 14B is a plot illustrating the relationship between the dissolution half-life and surface area of the angiogenesis inhibitor itraconazole in different dry powders.

FIG. 15 is a plot illustrating the relationship between the dissolution half-life and Cₘₐₓ of the angiogenesis inhibitor itraconazole in different dry powders.

FIG. 16 is a plot illustrating the relationship between the dissolution half-life and the dose adjusted Cₘₐₓ in different dry powders comprising the angiogenesis inhibitor itraconazole, expressed as a ratio to Formulation XIX.

FIG. 17 is a plot illustrating the cumulative percentage mass dissolution of the ISM collected post-aerosolization of itraconazole suspension formulations from a Micro Mist nebulizer at 15 L/min in the UniDose and then POD dissolution testing in a USP Apparatus II set-up.

FIG. 18 is a plot illustrating the cumulative mass percent of the recovered dose from different dry powders comprising the angiogenesis inhibitor itraconazole deposited on stage 4 of the cNGI.

FIG. 19 is a plot illustrating the relationship between the dissolution half-life and Cₘₐₓ of the angiogenesis inhibitor itraconazole in different dry powders.

FIG. 20 is a plot illustrating the relationship between the rate of diffusion and the dose adjusted Cₘₐₓ of different dry powders comprising the angiogenesis inhibitor itraconazole expressed as a ratio to Formulation XIX.

FIG. 21 is a plot illustrating the cumulative mass percent of the recovered dose from nebulized suspension formulations of the angiogenesis inhibitor itraconazole deposited on stage 4 of the cNGI.

FIGS. 22A and 22B are graphs showing the simulated kinetics of Formulation XIX, Formulation XII, and SPORANOX® in terms of Plasma Exposure (FIG. 22A) and Lung Exposure (FIG. 22B) using a model established from animal pharmacokinetic (PK) data and human data for SPORANOX®. In both simulations. 5 mg was inhaled once daily (Formulations XIX and XII), while 200 mg SPORANOX® oral solution dose was administered twice a day. The concentration of the angiogenesis inhibitor itraconazole was measured over seven days of dosing.

FIGS. 23A and 23B are graphs showing the simulated kinetics of Formulation XIX, Formulation XII, and SPORANOX® in terms of Plasma Exposure (FIG. 23A) and Lung Exposure (FIG. 23B) using a model established from animal PK data and human data for SPORANOX®. In both instances simulations 20 mg was inhaled once daily (Formulations XII and XIX), while 200 mg SPORANOX® oral solution dose was administered twice a day. The concentration of the angiogenesis inhibitor itraconazole was measured over seven days of dosing.

FIG. 24 is a graph showing the Single Dose Formulation XII plasma pharmacokinetic profile over 96 hours in healthy volunteers. Details of the study are provided in Example 21.

FIG. 25 is a graph showing the Formulation XII plasma pharmacokinetic profile over 24 hours after a single dose or after 14 daily doses in healthy volunteers. Details of the study are provided in Example 21.

FIGS. 26A and 26B are graphs showing summary data for systemic pharmacokinetics after a single inhaled or oral dose in asthma patients. Pharmacokinetic profiles of the angiogenesis inhibitor itraconazole in sputum (FIG. 26A) and plasma (FIG. 26B) following single doses of Formulation XII (▴) or oral SPORANOX® (Δ) administered to asthmatics.

### DETAILED DESCRIPTION OF THE INVENTION

The present disclosure relates to methods of treating cancer (e.g., lung cancer, e.g., NSCLC) by administering a respirable dry powder to a subject in need thereof by inhalation (e.g., oral inhalation), wherein the dry powder comprises homogenous respirable dry particles that comprise an angiogenesis inhibitor (e.g., itraconazole). In preferred aspects, the angiogenesis inhibitor is itraconazole (e.g., crystalline itraconazole). The dry powder may further comprise a stabilizer. In preferred aspects, the stabilizer is polysorbate 80 (PS80). The dry powder may further comprise one or more excipients. In preferred aspects, the one or more excipients are leucine and a sodium salt (e.g., sodium sulfate).

The inventors have discovered that administration of a dry powder disclosed herein can achieve a lung concentration of the angiogenesis inhibitor (e.g., itraconazole) that is substantially greater than those achievable by oral dosing. Without wishing to be bound by theory, it is believed that by achieving greater lung concentration of the angiogenesis inhibitor using the dry powder disclosed herein, the administration can be effective for treating cancer (e.g., lung cancer), while minimizing the systemic concentration of the angiogenesis inhibitor which can prevent side-effects and toxicity. It is further believed that a therapeutic concentration of the angiogenesis inhibitor in the lung can be achieved with a relatively low amount of total dose administered, e.g., relative to conventional methods of administration. For example, studies involving the treatment of NSCLC with oral itraconazole required doses of 600 mg/day, which is far greater than the standard recommended dose of itraconazole (about 200 mg/day).

Administering a dry powder disclosed herein can obtain a relatively high ratio of lung concentration:systemic concentration of the angiogenesis inhibitor (e.g., itraconazole). Without wishing to be bound by any particular theory, it is believed that a relatively high ratio of lung concentration:systemic concentration can provide effective treatment of the cancer while minimizing off-target effects and/or toxicity. Moreover, the ability to obtain a high ratio of drug in the lung provides the ability to administer relatively low amounts of the angiogenesis inhibitor to the subject, compared to the large amounts required for oral or intravenous dosing. Additionally, the inventors have discovered that the dry powders disclosed herein can be administered to achieve more consistent exposure of the angiogenesis inhibitor (e.g., over multiple doses, or in a group of subjects), relative to other formulations or routes of administration of angiogenesis inhibitor (e.g., as compared to an oral dose of itraconazole). As such, the methods disclosed herein provide an advantage over current applications of angiogenesis inhibitors, which are typically administered orally in large amounts, and which cannot achieve predictable or consistent concentrations in the cancerous tissue (e.g., lung tissue).

The dry powders disclosed herein may be administered to a subject by inhalation, such as oral inhalation. To achieve oral inhalation, a dry powder inhaler may be used, such as a passive dry powder inhaler. The dry powders disclosed herein can be used to treat cancer in a subject, such as lung cancer (e.g., NSCLC). An inhaled formulation of an angiogenesis inhibitor (e.g., itraconazole) minimizes many of the downsides of oral or intravenous (IV) formulations in treating these patients.

The respirable dry powders disclosed herein comprise an angiogenesis inhibitor. The angiogenesis inhibitor may be in crystalline particulate form. In preferred embodiments, the angiogenesis inhibitor is itraconazole in crystalline particulate form. Respirable dry powders comprising itraconazole for use in treating a fungal infection have been described in WO 2018/071757, WO 2019/204583, and WO 2019/204597, the entire contents of which are incorporated herein by reference in their entireties.

The inventors have discovered that dry powders that comprise itraconazole in amorphous form have shorter lung residence times, reduced lung to plasma exposure ratios and undesirable toxic effects on lung tissue when inhaled at therapeutic doses. Without wishing to be bound by any particular theory, it is believed that the dry powders disclosed herein comprising crystalline forms (e.g., nanocrystalline forms) of an angiogenesis inhibitor (e.g., itraconazole) have a slower dissolution rate in the lung relative to the amorphous form, providing more continuous exposure over a 24 hour period after administration and minimizing systemic exposure.

In addition, the observed local toxicity in lung tissue without amorphous dosing is not related to the total exposure of the lung tissue to itraconazole, in terms of total dose or duration of exposure. For example, itraconazole has no known activity against human or animal lung cells and so increasing local concentration has no local pharmacological activity to explain the local toxicity. Instead, the toxicity of the amorphous form appears related to the increased solubility secondary to the amorphous nature of the itraconazole, resulting in supersaturation of the itraconazole in the interstitial space and the resultant recrystallization in the tissue leading to local, granulomatous inflammation. Surprisingly, the inventors discovered that the dry powders disclosed herein that comprise itraconazole in crystalline particulate form are less toxic to lung tissue, relative to more rapidly dissolving formulations such as those comprising amorphous itraconazole. This was surprising because crystalline particulate itraconazole formulations have a lower aqueous solubility, and consequently a slower dissolution rate, in comparison to the formulations comprising amorphous itraconazole, and remain in the lung longer than a corresponding dose of itraconazole in amorphous form. Furthermore, the crystalline particulate itraconazole also results in higher lung exposure after a single dose and over 28 days than a corresponding dose of the itraconazole in amorphous form.

The crystallinity of the angiogenesis inhibitor, as well as the size of the angiogenesis inhibitor crystalline sub-particles, and the identity and amount of excipients and stabilizers, all appear to be important factors for effective therapy and for reduced toxicity in the lung. Without wishing to be bound by any particular theory, it is believed that smaller crystalline particles of the angiogenesis inhibitor (e.g., nano-crystalline or micro-crystalline angiogenesis inhibitor) will dissolve in the airway lining fluid more rapidly than larger crystalline particles—in part due to the larger total amount of surface area. It is also believed that crystalline angiogenesis inhibitor will dissolve more slowly in the airway lining fluid than the amorphous angiogenesis inhibitor, in part due to the lower aqueous solubility. Accordingly, the dry powders described herein can be formulated using angiogenesis inhibitor (e.g., itraconazole) in crystalline particulate form that provides for an angiogenesis inhibitor in a desired crystalline size or range of crystalline sizes within the dry powders, and optionally with suitable excipients and stabilizers in a suitable ratio with the angiogenesis inhibitor, each of which can be tailored to affect, e.g., dissolution rate, and achieve desired pharmacokinetic properties while avoiding unacceptable toxicity in the lungs.

The respirable dry powders used in the methods disclosed herein may include homogenous respirable dry particles that comprise 1) an angiogenesis inhibitor (e.g., itraconazole, such as itraconazole in crystalline particulate form), 2) a stabilizer, and optionally 3) one or more excipients. Such respirable dry particles can be prepared using any suitable method, such as by preparing a feedstock in which an angiogenesis inhibitor (e.g., itraconazole, such as itraconazole in crystalline particulate form) is suspended in an aqueous solution of excipients, and spray drying the feedstock.

The dry powders may be administered to a subject by inhalation, such as oral inhalation. To achieve oral inhalation, a dry powder inhaler may be used, such as a passive dry powder inhaler. The dry powders can be used to treat cancer in a subject, such as lung cancer, or more specifically, non-small cell lung cancer (NSCLC). The inhaled formulations of angiogenesis inhibitor (e.g., itraconazole) disclosed herein minimize many of the downsides of oral or intravenous (IV) formulations in treating these subjects. For example, oral or intravenously administered formulations of angiogenesis inhibitor may not consistently achieve therapeutic concentrations at the site of the cancer cells or tumor (e.g., in the lung), or may require a relatively high dose to achieve therapeutic lung concentrations, resulting in toxicity or side-effects. The high doses required by oral administration also increase the risk of drug-drug interactions (DDIs). For example, administering oral angiogenesis inhibitor (e.g., itraconazole) in combination with a standard of care protocol for treating cancer may be contraindicated due to DDIs, such as when the standard of care involves administering a cancer drug that is a substrate for the same enzyme as the angiogenesis inhibitor, e.g., a cytochrome P450, such as CYP3A4. Thus, the high doses required for oral administration of the angiogenesis inhibitor can limit its use in combination with other cancer drugs or its use as a neoadjuvant therapy. The dry powders disclosed herein can reliably achieve therapeutic concentrations of an angiogenesis inhibitor (e.g., itraconazole) in a subject (e.g., in lung tissue), which can lower the total dose of angiogenesis inhibitor needed to be administered to the subject, and also achieve relatively low systemic concentrations (e.g., in plasma) of the angiogenesis inhibitor, which can reduce side-effects and toxicity whilst having efficacy (e.g., in the treatment of a cancer, such as lung cancer). Advantageously, the relatively lower amount of angiogenesis inhibitor that is needed using a dry powder disclosed herein, compared to the large amounts required for oral dosing, can reduce the risk of DDIs, which provides the opportunity to combine the dry powder comprising an angiogenesis inhibitor with another drug, e.g., a standard of care treatment for NSCLC, such as using a dry powder disclosed herein as a neoadjuvant therapy, with a lower risk of DDIs.

An inhaled formulation of angiogenesis inhibitor (e.g., itraconazole) disclosed herein may provide synergistic effects when combined with another chemotherapeutic. As such, the present disclosure outlines methods comprising a combination therapy. For example, when a respirable dry powder disclosed herein is administered to a subject in addition to another therapy (e.g., an additional therapeutic agent, and/or radiation), the extent of tumor volume reduction in the subject may be improved relative to the extent of tumor volume reduction when administering the same treatment without the dry powder.

### Definitions

As used herein, the term “about” refers to a relative range of plus or minus 5% of a stated value, e.g., “about 20 mg” would be “20 mg plus or minus 1 mg”.

As used herein, the terms “administration” or “administering” refer to the introduction of a composition comprising a therapeutic agent to a subject. For example, administering may refer to introducing respirable dry particles disclosed herein to the respiratory tract of a subject.

As used herein, the term “amorphous” indicates lack of significant crystallinity when analyzed via powder X-ray diffraction (XRD).

The term “capsule emitted powder mass” or “CEPM” as used herein refers to the amount of dry powder emitted from a capsule or dose unit container during actuation from the dry powder inhaler, such as during an inhalation maneuver. CEPM is measured gravimetrically, typically by weighing a capsule before and after the emission event to determine the mass of powder removed. CEPM can be expressed either as the mass of powder removed, in milligrams, or as a percentage of the initial filled powder mass in the capsule prior to the emission event.

The term “crystalline particulate form” as used herein refers to an angiogenesis inhibitor (e.g., itraconazole), including pharmaceutically acceptable forms thereof including salts, hydrates, enantiomers as the like, that is in the form of a particle (i.e., sub-particle that is smaller than the respirable dry particles that comprise the dry powders disclosed herein) and in which the angiogenesis inhibitor is at least about 50% crystalline. The percent crystallinity of an angiogenesis inhibitor refers to the percentage of the compound that is in crystalline form relative to the total amount of compound present in the sub-particle. If desired, the angiogenesis inhibitor can be at least about 60%, at least about 70%, at least about 80%, at least about 90%, at least about 95%, or about 100% crystalline. An angiogenesis inhibitor in crystalline particulate form is in the form of a particle that is about 50 nanometers (nm) to about 5,000 nm volume median diameter (Dv50), preferably 80 nm to 1750 nm Dv50, or preferably 50 nm to 800 nm Dv50.

The term “dispersible” is a term of art that describes the characteristic of a dry powder or respirable dry particles to be dispelled into a respirable aerosol. Dispersibility of a dry powder or respirable dry particles is expressed herein, in one aspect, as the quotient of the volumetric median geometric diameter (VMGD) measured at a dispersion (i.e., regulator) pressure of 1 bar divided by the VMGD measured at a dispersion (i.e., regulator) pressure of 4 bar, or VMGD at 0.5 bar divided by the VMGD at 4 bar as measured by laser diffraction, such as with a HELOS/RODOS. These quotients are referred to herein as “1 bar/4 bar dispersibility ratio” and “0.5 bar/4 bar dispersibility ratio”, respectively, and dispersibility correlates with a low quotient. For example, 1 bar/4 bar dispersibility ratio refers to the VMGD of a dry powder or respirable dry particles emitted from the orifice of a RODOS dry powder disperser (or equivalent technique) at about 1 bar, as measured by a HELOS or other laser diffraction system, divided by the VMGD of the same dry powder or respirable dry particles measured at 4 bar by HELOS/RODOS. Thus, a highly dispersible dry powder or respirable dry particles will have a 1 bar/4 bar dispersibility ratio or 0.5 bar/4 bar dispersibility ratio that is close to 1.0. Highly dispersible powders have a low tendency to agglomerate, aggregate or clump together and/or, if agglomerated, aggregated or clumped together, are easily dispersed or de-agglomerated as they emit from an inhaler and are breathed in by a subject. In another aspect, dispersibility is assessed by measuring the particle size emitted from an inhaler as a function of flowrate. As the flow rate through the inhaler decreases, the amount of energy in the airflow available to be transferred to the powder to disperse it decreases. A highly dispersible powder will have a size distribution such as is characterized aerodynamically by its mass median aerodynamic diameter (MMAD) or geometrically by its VMGD that does not substantially increase over a range of flow rates typical of inhalation by humans, such as about 15 to about 60 liters per minute (LPM), about 20 to about 60 LPM, or about 30 LPM to about 60 LPM. A highly dispersible powder will also have an emitted powder mass or dose, or a capsule emitted powder mass or dose, of about 80% or greater even at the lower inhalation flow rates. VMGD may also be called the volume median diameter (VMD), ×50, or Dv50.

The term “dry particles” as used herein refers to respirable particles that may comprise up to about 15% total of water and/or another solvent. Preferably, the dry particles comprise water and/or another solvent up to about 10% total, up to about 5% total, up to about 1% total, or between 0.01% and 1% total, by weight of the dry particles, or can be substantially free of water and/or other solvent.

The term “dry powder” as used herein refers to compositions that comprise respirable dry particles. A dry powder may comprise up to about 15% total of water and/or another solvent. Preferably the dry powder comprise water and/or another solvent up to about 10% total, up to about 5% total, up to about 1% total, or between 0.01% and 1% total, by weight of the dry powder, or can be substantially free of water and/or other solvent. In one aspect, the dry powder is a respirable dry powder.

The term “effective amount,” as used herein, refers to the amount of agent needed to achieve the desired effect: such as treating a cancer, e.g., lung cancer, such as non-small cell lung cancer. The actual effective amount for a particular use can vary according to the particular dry powder or respirable dry particle, the mode of administration, and the age, weight, general health of the subject, and severity of the symptoms or condition being treated. Suitable amounts of dry powders and dry particles to be administered, and dosage schedules for a particular patient can be determined by a clinician of ordinary skill based on these and other considerations.

As used herein, the term “emitted dose” or “ED” refers to an indication of the delivery of a drug formulation from a suitable inhaler device after a firing or dispersion event. More specifically, for dry powders, the ED is a measure of the percentage of powder that is drawn out of a unit dose package and that exits the mouthpiece of an inhaler device. The ED is defined as the ratio of the drug or powder delivered by an inhaler device to the nominal dose (i.e., the mass of drug or powder per unit dose placed into a suitable inhaler device prior to firing). The ED is an experimentally-measured parameter, and can be determined using the method of USP Section 601 Aerosols, Metered-Dose Inhalers and Dry Powder Inhalers. Delivered-Dose Uniformity, Sampling the Delivered Dose from Dry Powder Inhalers, United States Pharmacopeia convention, Rockville, MD, 13^{th} Revision. 222-225, 2007. This method utilizes an in vitro device set up to mimic patient dosing. It can also be calculated from the results generated by Next Generation Impactor (NGI) experiments, through summation of all of the drug or powder assayed from the mouthpiece adapter, NGI induction port, and all of the stages within the NGI. The results generated through ED testing per USP 601 and the results generated via the NGI are typically in good agreement.

The term “lung to plasma ratio” or “lung:plasma ratio” refers to the ratio of a concentration of an angiogenesis inhibitor (e.g., itraconazole) in the lung versus the concentration of the angiogenesis inhibitor in the plasma at either a specific point in time or over a specific range of time. For example, the lung:plasma ratio may be calculated based on concurrent measurements at the maximum concentration (i.e., the “Cₘₐₓ”) of the angiogenesis inhibitor in the lung or in the serum, or at any point in time. The lung:plasma ratio may also be calculated for a total exposure over a certain period of time (i.e., the “area under the curve” or “AUC”) such as over a 24 hour period. The lung concentrations of the angiogenesis inhibitor may be assessed by measuring the levels in the sputum, by lung lavage, by biopsy or by some other method. The lung:plasma ratio may be calculated based on concurrent measurements at any point in the dosing cycle and may be calculated based on concurrent measurements before or at steady state.

The term “nominal dose” as used herein refers to an individual dose of an angiogenesis inhibitor (e.g., itraconazole). The nominal dose is the total dose of the angiogenesis inhibitor (e.g., itraconazole) within one receptacle, e.g., capsule, blister, or ampule.

The terms “FPF (<X),” “FPF (<X microns),” and “fine particle fraction of less than X microns” as used herein, wherein X equals, for example, 3.4 microns, 4.4 microns, 5.0) microns or 5.6 microns, refer to the fraction of a sample of dry particles that have an aerodynamic diameter of less than X microns. For example, FPF (<X) can be determined by dividing the mass of respirable dry particles deposited on stage two and on the final collection filter of a two-stage collapsed Andersen Cascade Impactor (ACI) by the mass of respirable dry particles weighed into a capsule for delivery to the instrument. This parameter may also be identified as “FPF_TD(<X),” where TD means total dose. A similar measurement can be conducted using an eight-stage ACI. An eight-stage ACI cutoffs are different at the standard 60 L/min flowrate, but the FPF_TD(<X) can be extrapolated from the eight-stage complete data set. The eight-stage ACI result can also be calculated by the USP method of using the dose collected in the ACI instead of what was in the capsule to determine FPF. Similarly, a seven-stage Next Generation Impactor (NGI) can be used.

The terms “FPD (<X)”, “FPD <X microns”, FPD(<X microns)” and “fine particle dose of less than X microns” as used herein, wherein X equals, for example, 3.4 microns, 4.4 microns, 5.0 microns or 5.6 microns, refer to the mass of a therapeutic agent delivered by respirable dry particles that have an aerodynamic diameter of less than X micrometers. FPD <X microns can be determined by using an eight-stage Andersen Cascade Impactor (ACI) or a Next Generation Impactor (NGI) at the standard 60 L/min flowrate and summing the mass deposited on the final collection filter, and either directly calculating or extrapolating the FPD value. Similarly, a seven-stage Next Generation Impactor (NGI) can be used.

The term “respirable” as used herein refers to dry particles or dry powders that are suitable for delivery to the respiratory tract (e.g., pulmonary delivery) in a subject by inhalation. Respirable dry powders or dry particles have a mass median aerodynamic diameter (MMAD) of less than about 10 microns, preferably about 5 microns or less.

As used herein, the term “respiratory tract” includes the upper respiratory tract (e.g., nasal passages, nasal cavity, throat, pharynx, and larynx), respiratory airways (e.g., trachea, bronchi, and bronchioles) and lungs (e.g., respiratory bronchioles, alveolar ducts, alveolar sacs, and alveoli).

As used herein, the term “lower respiratory tract” includes the respiratory airways and lungs.

The term “small” as used herein to describe respirable dry particles refers to particles that have a volume median geometric diameter (VMGD) of about 10 microns or less, preferably about 5 microns or less, or less than 5 microns.

The term “stabilizer” as used herein refers to a compound that improves the physical stability of angiogenesis inhibitor in crystalline particulate form when suspended in a liquid in which the angiogenesis inhibitor is poorly soluble (e.g., reduces the aggregation, agglomeration, Ostwald ripening and/or flocculation of the particulates). Suitable stabilizers are surfactants and amphiphilic materials and include Polysorbates (PS: polyoxyethylated sorbitan fatty acid esters), such as polysorbate 20 (PS20), polysorbate 40 (PS40), polysorbate 60 (PS60), and polysorbate 80 (PS80); fatty acids such as lauric acid, palmitic acid, myristic acid, oleic acid and stearic acid; sorbitan fatty acid esters, such as Span20, Span40, Span60, Span80, and Span 85; phospholipids such as dipalmitoylphosphosphatidylcholine (DPPC), 1,2-dipalmitoyl-sn-glycero-3-phospho-L-serine (DPPS), 1,2-dipalmitoyl-sn-glycero-3-phosphocholine (DSPC), 1-palmitoyl-2-oleoylphosphatidylcholine (POPC), and 1,2-dioleoyl-sn-glycero-3-phosphocholine (DOPC); phosphatidylglycerols (PGs) such as diphosphatidyl glycerol (DPPG), DSPG, DPPG, POPG, etc.; 1,2-distearoyl-sn-glycero-3-phosphoethanolamine (DSPE); fatty alcohols; benzyl alcohol, polyoxyethylene-9-lauryl ether; glycocholate; surfactin; poloxomers; polyvinylpyrrolidone (PVP); PEG/PPG block co-polymers (Pluronics/Poloxamers); polyoxyethyene chloresteryl ethers; POE alky ethers; tyloxapol; lecithin; and the like. Preferred stabilizers are polysorbates and fatty acids. A particularly preferred stabilizer is polysorbate 80 (PS80). Another preferred stabilizer is oleic acid, or a salt thereof.

The term “homogenous dry particles” as used herein refers to particles that are compositionally homogenous. Homogenous dry particles disclosed herein are substantially the same in their composition of angiogenesis inhibitor (e.g., itraconazole, such as crystalline itraconazole), stabilizer, and optionally one or more excipients, and exclude a blend of two or more particles.

### Therapeutic Use and Methods

The present disclosure relates to a method of treating cancer (e.g., lung cancer, such as NSCLC), the method comprising administering to the respiratory tract of a subject in need thereof a dry powder, wherein the dry powder comprises homogenous respirable dry particles that comprise an angiogenesis inhibitor (e.g., itraconazole), and optionally a stabilizer (e.g., polysorbate 80) and/or one or more excipients (e.g., leucine and sodium sulfate).

The methods disclosed herein are especially useful in treating a subject with lung cancer, such as non-small cell lung cancer (NSCLC). The NSCLC can comprise an adenocarcinoma, squamous cell carcinoma, or large cell carcinoma. The subject may be chemotherapy-naïve (e.g., a subject that has not previously received any chemotherapy). Alternatively, the subject may have previously received one or more cancer therapies (e.g., chemotherapy), and may be resistant or refractory to the therapy. In other words, the methods disclosed herein may be used to treat resistant or refractory lung cancer. For example, a method disclosed herein may comprise administering a dry powder to the respiratory tract of a subject with a cancer that has not responded to one or more prior therapies, or has become resistant to one or more prior therapies. A cancer treated using a method disclosed herein may be a relapsed or recurrent cancer, e.g., a cancer that has returned after a period of remission following a prior treatment of the cancer. In preferred aspects, the subject is a human.

The methods disclosed herein can be used to treat a primary cancer or a metastatic cancer. The cancer (e.g., lung cancer) may be an early-stage cancer, a locally advanced cancer, or advanced cancer. The cancer may be at any stage, including stage 0, stage I, stage II, stage III (including stage IIIA and IIIB), or stage IV (including stage IVA and IVB). For example, a method disclosed herein may be used to treat a subject with stage 0 NSCLC, stage I NSCLC, stage II NSCLC, stage IIIA NSCLC, stage IIIB NSCLC, stage IVA NSCLC, or stage IVB NSCLC. In some embodiments, the cancer is advanced, metastatic, and/or refractory cancer (e.g., advanced, metastatic, and/or refractory NSCLC). In some embodiments, the method is to treat only a primary cancer (e.g., where the method is not intended to treat a metastatic cancer).

A cancer treated using a method disclosed herein may be linked to one or more of the following: EGFR mutations (e.g., sensitizing EGFR mutations). KRAS mutations, ALK rearrangement, ROS1 rearrangement, BRAF V600E mutation, NTRK gene fusion, MET exon 14 skipping, RET rearrangement, or PD-L1.

A method of treatment disclosed herein can inhibit cancer cell growth or tumor growth. e.g., of a lung cancer such as NSCLC. For example, administering a therapeutically effective amount of a dry powder disclosed herein to the respiratory tract of a subject (optionally in combination with an additional therapeutic agent and/or radiation) can inhibit cell growth or tumor growth by at least about 20%, e.g., about 30%, about 40%, about 50%, about 60%, about 70%, about 80%, about 90%, or more, relative to an untreated subject, or a subject that is not treated with a dry powder disclosed herein. A method of treatment disclosed herein can result in tumor regression that can be observed for a period of at least about 10 days, about 20 days, about 30 days, about 2 months, about 4 months, about 6 months, about 1 year, or longer.

A method disclosed herein may increase the duration of survival of the subject treated. For example, administering a therapeutically effective amount of a dry powder disclosed herein to the respiratory tract of a subject (optionally in combination with an additional therapeutic agent and/or radiation) can increase the duration of survival of the subject by at least 1 month when compared to another subject that did not receive treatment, or when compared to a subject that received treatment not including a dry powder disclosed herein. In some embodiments, duration of the survival is increased by at least 2 months, at least 3 months, at least 4 months, at least 5 months, at least 6 months, or longer.

A method disclosed herein may increase the progression free of survival of the subject treated. For example, administering a therapeutically effective amount of a dry powder disclosed herein to the respiratory tract of a subject (optionally in combination with an additional therapeutic agent and/or radiation) can increase the progression free of survival of the subject by at least 1 month when compared to another subject that did not receive treatment, or when compared to a subject that received treatment not including a dry powder disclosed herein. In some embodiments, progression free of survival is increased by at least 2 months, at least 3 months, at least 4 months, at least 5 months, at least 6 months, or longer.

A method disclosed herein may increase the response rate in a group of subjects, when compared to another group that did not receive treatment, or a group that received treatment not including a dry powder disclosed herein. In some embodiments, the response rate is increased by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 50%, or greater.

Methods disclosed herein may improve a symptom of cancer (e.g., lung cancer) in a subject with cancer, e.g., lung cancer such as NSCLC. For example, a method disclosed herein may improve one or more of the following symptoms in a subject: breathing difficulty or shortness of breath, coughing (e.g., persistent cough or chronic cough), blood in sputum, pain (e.g., chest pain or back pain), lung infection, jaundice, bloating or the feeling of a full stomach, headaches, dizziness, seizures, weakness or numbness (such as weakness or numbness in limbs), fatigue, and unexplained weight loss.

Methods disclosed herein may comprise administration in the form of an induction treatment, a neoadjuvant treatment, or adjuvant treatment, such as being carried out prior to, or following, a surgery to treat the cancer. For example, a method disclosed herein may comprise administering the dry powder to the respiratory tract of the subject before or after performing surgery to remove cancer tissue from the subject. The surgery may be any surgery typically performed to treat cancer, particularly lung cancer, such as a segmentectomy, a sleeve resection, a wedge resection, a lobectomy, a pneumonectomy, a lymphadenectomy, or a combination thereof. For example, prior to undergoing surgery to remove cancer tissue from the lung, the subject may receive one or more doses of a dry powder disclosed herein (either alone or as a combination therapy disclosed herein) at least 1 day before the surgery, e.g., at least 3 days, at least 1 week, at least 2 weeks, at least 3 weeks, at least 4 weeks, at least 1 month, at least 2 months, at least 3 months, or at least 4 months, or longer, before the surgery. The subject may receive multiple doses of the dry powder, e.g., over a period of time ending immediately prior to surgery. Alternatively, or in addition to the foregoing, after undergoing surgery to remove cancer tissue from the lung, the subject may receive one or more doses of a dry powder disclosed herein (either alone or as a combination therapy disclosed herein) at least 1 day after the surgery, e.g., at least 3 days, at least 1 week, at least 2 weeks, at least 3 weeks, at least 4 weeks, at least 1 month, at least 2 months, at least 3 months, or at least 4 months, or longer, after the surgery. In particular, the methods disclosed herein may be used as maintenance therapy, and/or to prevent recurrence of disease.

### Combination Therapies

A method disclosed herein can comprise a combination therapy. In particular, if desired or indicated, the dry powders described herein can be administered with one or more other therapeutic agents, e.g., an additional therapeutic agent disclosed herein. The other therapeutic agent can be administered by any suitable route, described in more detail below. The dry powder can be administered before, substantially concurrently with, or subsequent to administration of the other therapeutic agent. Preferably, the dry powder and the additional therapeutic agent are administered so as to provide substantial overlap of their pharmacologic activities.

For example, the method may comprise administering a dry powder disclosed herein to a subject, in addition to one or more therapeutic agents and/or radiation. The additional therapeutic agent may be any suitable agent used in the treatment of a cancer, e.g., any chemotherapy used to treat lung cancer that is known in the art. For example, a dry powder disclosed herein may be administered to a subject that is treated with a standard-of-care treatment outlined in the National Comprehensive Cancer Network (NCCN) Clinical Practice Guidelines in Oncology. The combination therapy may comprise administering the dry powder in addition to chemoradiation. The combination therapy may comprise administering the dry powder in addition to platinum-doublet chemotherapy. The additional therapeutic agent can be a chemotherapeutic drug, a targeted cancer therapy drug, an immunotherapy drug, or a combination thereof.

The combination therapy may comprise administering a dry powder disclosed herein, in addition to an oral dose of itraconazole, and optionally one or more additional therapeutic agents. For example, a method disclosed herein may comprise administering to the subject a dry powder comprising itraconazole, in addition to an oral dose of itraconazole, and optionally one or more additional therapeutic agents (e.g., a chemotherapy), such as a standard-of-care treatment for lung cancer.

The additional therapeutic agent and/or oral itraconazole may be administered to the subject at or about the time the dry powder is administered to the subject. For example, a dose of the additional therapeutic agent and/or oral itraconazole may be administered no more than 24 hours before or after administering a dose of the dry powder, e.g., within about 12 hours, within about 6 hours, within about 4 hours, within about 3 hours, within about 2 hours, within about 1 hour, or within about 30 minutes, or less, of administering the dry powder. However, the additional therapeutic agent and/or oral itraconazole does not need to be administered at or about the time as the dry powder is administered. For example, the dry powder may be administered to the subject more than a day before or a day after administering the additional chemotherapeutic and/or oral itraconazole, e.g., 2 days before or after, 3 days before or after, 4 days before or after, 5 days before or after, 6 days before or after, 1 week before or after, 2 weeks before or after, 3 weeks before or after, 4 weeks before or after, or longer, depending, e.g., on the desired outcome of treatment, or needs of the subject. In some embodiments, the dry powder is administered to the subject between about 1 and about 28 days before administering an additional chemotherapeutic and/or oral itraconazole, e.g., 1 to about 21 days, about 1 to about 18 days, about 1 to about 16 days, about 1 to about 14 days, about 1 to about 12 days, or about 1 to about 10 days, before administering the additional chemotherapeutic and/or oral itraconazole.

An additional therapeutic agent used in a combination therapy disclosed herein may be any suitable agent known in the art for treating cancer, e.g., lung cancer. For example, the therapeutic agent may be selected from the group consisting of platinum-based drugs, antimetabolites, antimicrotubule agents, topoisomerase inhibitors, anthracyclines, KRAS inhibitors, ALK inhibitors, EGFR inhibitors, VEGF inhibitors, BRAF inhibitors, MEK inhibitors, RET inhibitors, MET inhibitors, and immunotherapy drugs (e.g., immune checkpoint inhibitors, e.g., PD-1/PD-L1 inhibitors or CTLA-4 inhibitors), or the like.

For example, the additional therapeutic agent may be selected from the group consisting of pemetrexed, cisplatin, carboplatin, oxaliplatin, satraplatin, picoplatin, nedaplatin, triplatin, lipoplatin, paclitaxel (including albumin-bound paclitaxel), docetaxel, doxorubicin, gemcitabine, vinblastine, vinorelbine, etoposide, SN-38, camptothecin, topotecan, exatecan, irinotecan, belotecan, methotrexate, bevacizumab (AVASTIN®), ranibizumab, ramucirumab (CYRAMZA®), nintedanib, aflibercept, erlotinib (TARCEVA®), afatinib (GILOTRIF®), axitinib, gefitinib (IRESSA®), cabozantinib (COMETRIQ®, CABOMETYX®), osimertinib (TAGRISSO®), dacomitinib (VIZIMPRO®), sotorasib, crizotinib (XALKORI®), entrectinib (ROZLYTREK®), lenvatinib, pazopanib, ceritinib (ZYKADIA®), alectinib (ALECENSA®), brigatinib (ALUNBRIG®), lorlatinib (LORBRENA®), dabrafenib (TAFINLAR®), regorafenib, vemurafenib (ZELBORAF®), sorafenib, sunitinib, everolimus, thalidomide, lenalidomide, trametinib (MEKINIST®), vandetanib (CAPRELSA®), selpercatinib (RETEVMO™), pralsetinib (GAVRETO™), capmatinib (TABRECTA™), tepotinib (TEPMETKO®), larotrectinib (VITRAKVI®), amivantamab, mobocertinib, nivolumab (OPDIVO®), ipilimumab (YERVOY®), atezolizumab (TECENTRIQ®), cetuximab (ERBITUX®), pembrolizumab (KEYTRUDA®), tremelimumab, cemiplimab (LIBTAYO®), pidilizumab, durvalumab (IMFINZI®), necitumumab, oral or intravenous itraconazole, and combinations thereof.

The additional therapeutic agent may be a compound disclosed in any one of U.S. Pat. Nos. 5,997,318, 6,051,227, 6,682,736, 6,808,710, 6,984,720, 7,034,121, 7,169,901, 7,297,334, 7,423,125, 7,488,802, 7,605,238, 7,943,743, 8,008,449, 8,034,905, 8,168,757, 8,354,509, 8,609,089, 8,686,119, 8,779,105, 8,779,108, 8,900,587; U.S. Patent Publication Nos. US 2012/263677, US 2014/0356353 or US 2021/0324106; or PCT Publication Nos. WO 2007/113648, WO 2012/145493, WO 2012/122444, WO 2013/014668, WO 2013/173223, each of which are incorporated herein by reference in their entireties.

Non-limiting examples of standard-of-care treatment regimens that may be used in a combination therapy disclosed herein (e.g., combined with a dry powder) include the following; carboplatin or cisplatin, pemetrexed, and pembrolizumab; carboplatin, paclitaxel, and atezolizumab (optionally with bevacizumab); carboplatin, albumin-bound paclitaxel, and atezolizumab; nivolumab and ipilimumab; carboplatin or cisplatin, pemetrexed, nivolumab, and ipilimumab; carboplatin, paclitaxel, and optionally bevacizumab; carboplatin or cisplatin, pemetrexed, and optionally bevacizumab; cisplatin and another chemotherapy; carboplatin and another chemotherapy; gemcitabine and docetaxel or vinorelbine; a single agent chemotherapy; carboplatin, paclitaxel, and pembrolizumab; carboplatin, albumin-bound paclitaxel, and pembrolizumab; or carboplatin, paclitaxel, nivolumab, and ipilimumab. It will be understood by a person of skill in the art that one or more of the aforementioned therapeutic agents may be substituted with another, or excluded. e.g., depending on the subject's condition, disease status, or response to the therapy.

The additional therapeutic agent can be administered to the subject by any suitable route of administration. Non-limiting examples of suitable routes of administration include oral, intravenous, intramuscular, inhalation (e.g., intrabronchial, intranasal, oral inhalation, intranasal drops), intraarterial, intrathecal, intralymphatic, intralesional, intracapsular, intraorbital, intracardiac, intradermal, transtracheal, subcuticular, intraarticular, subcapsular, subarachnoid, subcutaneous, intraperitoneal, intraspinal, epidural, intrasternal, intratumoral, topical, epidermal, mucosal, intranasal, vaginal, rectal, or sublingual.

The additional therapeutic agent and/or oral itraconazole can be administered to the subject in any suitable dose, which may be the approved dose of that therapeutic agent and/or for the particular type of cancer or stage of cancer. However, it will be understood that the dose of the compounds or compositions of the present disclosure will be decided by the attending physician within the scope of sound medical judgment. The specific therapeutically effective dose level for any particular subject or organism will depend upon a variety of factors including, without limitation; the disease or disorder being treated, and the severity of the disease or disorder being treated; the activity of the specific compound or composition being administered; the specific compound or composition being administered; the age, body weight, general heath, sex, and/or diet of the subject; the time of administration, route of administration, and rate of excretion or metabolism of the specific compound or composition being administered; the duration of treatment, drugs used in combination or coincidental with the specific compound or composition being administered; and like factors known in the art. It will also be understood that the attending physician can determine or adjust the dose, such as lowering the dose of additional therapeutic agent and/or oral itraconazole when combining with a dry powder disclosed herein. Similarly, the total amount of dry powder administered, or total drug load in the dry powder administered, may be adjusted (e.g., lowered) when combined with one or more additional therapeutic agents and/or oral itraconazole.

Methods of treating a cancer disclosed herein may also comprise radiation therapy. For example, the method can comprise administering a dry powder disclosed herein to the respiratory tract of a subject with cancer (optionally in combination with one or more additional therapeutic agents disclosed herein) before or after performing radiation therapy to treat the cancer. The radiation therapy may be any suitable radiation therapy for treating cancer, particularly lung cancer, such as brachytherapy, external beam radiation therapy (EBRT), stereotactic body radiation therapy (SBRT), stereotactic ablative radiotherapy (SABR), three-dimensional conformal radiation therapy (3D-CRT), intensity modulated radiation therapy (IMRT), proton therapy, volumetric modulated arc therapy (VMAT), stereotactic radiosurgery (SRS), radiofrequency ablation, or a combination thereof.

### Pharmacokinetics, Dosing, and Other Considerations

The amount of dry powder administered to the subject may be sufficient to maintain a steady state concentration. As used herein, steady state concentration (Css) refers to the concentration of a drug, in for example lung or plasma, at the time a “steady state” has been achieved, and rates of drug administration and drug elimination are equal. Steady state concentration is a value approached as a limit and is achieved, theoretically, following the last of an infinite number of equal doses given at equal intervals. The maximum value under such conditions (Css,max) is given by Css,max=C0/(1−f), for a drug eliminated by first-order kinetics from a single compartment system. The ratio Css, max/C0 indicates the extent to which drug accumulates under the conditions of a particular dose regimen of, theoretically, an infinitely long duration; the corresponding ratio 1/(1−f) is sometimes called the Accumulation Ratio, R. Css is also the limit achieved, theoretically, at the “end” of an infusion of infinite duration, at a constant rate.

In some aspects, about 2 mg, about 3 mg, about 4 mg, 5 mg, about 10 mg, about 15 mg, about 20 mg, about 25 mg, about 30 mg, about 35 mg, about 40 mg, about 50 mg, about 2 mg to about 35 mg, about 5 mg to about 50 mg, about 10 mg to about 50 mg, about 15 mg to about 50 mg, nominal doses (of the angiogenesis inhibitor, e.g., itraconazole) may be administered. The dose and dosing regimen may be selected to achieve a certain lung; plasma ratio of the angiogenesis inhibitor, or to achieve certain steady state concentrations of the angiogenesis inhibitor in the lung and/or plasma.

The lung; plasma ratio may be at least about 100:1, at least about 200:1, at least about 300:1, at least about 400:1, at least about 500:1, at least about 600:1, at least about 700:1, at least about 800:1, at least about 1000:1, at least about 1300:1, at least about 1600:1, at least about 1900:1, at least about 2200:1, at least about 2500:1, at least about 2800:1, at least about 3000:1, at least about 3200:1, at least about 3400:1, at least about 3600:1, between 3000:1 to 4000:1, between 3500:1 to 4000:1, or between 3600:1 to 3700:1. Additionally, the lung:plasma ratio may be at least about 2:1, at least 3:1, at least 4:1, at least 5:1, at least 6:1, at least 7:1, at least 8:1, at least 9:1, at least 10:1, at least 15:1, at least 20:1, at least 25:1, at least 50:1, or at least 75:1. The lung:plasma ratio may be calculated based on concurrent measurements at the maximum concentration (i.e., the “Cₘₐₓ”) of the angiogenesis inhibitor in the lung or in the serum, or at any point in time. The lung; plasma ratio may also be calculated for a total exposure over a certain period of time (i.e., the “area under the curve” or “AUC”) such as over a 24 hour period. The lung; plasma ratio may be calculated based on concurrent measurements at any point in the dosing cycle and may be calculated before or at steady state.

At steady state, the lung:plasma ratio may be at least about 20:1, at least about 25:1, at least 50:1, at least 75:1, at least about 100:1, at least about 200:1, at least about 300:1, at least about 400:1, at least about 500:1, at least about 600:1, at least about 700:1, at least about 800:1, at least about 1000:1, at least about 1300:1, at least about 1600:1, at least about 1900:1, at least about 2200:1, at least about 2500:1, at least about 2800:1, at least about 3000:1, at least about 3200:1, at least about 3400:1, at least about 3600:1, between 3000:1 to 4000:1, between 3500:1 to 4000:1, or between 3600:1 to 3700:1.

A dry powder disclosed herein may be administered to achieve a certain plasma concentration of the angiogenesis inhibitor (e.g., itraconazole). The plasma concentration may be less than 40 ng/ml, less than 35 ng/ml, less than 30 ng/ml, less than 25 ng/ml, less than 20 ng/mL, less than 15 ng/ml, less than 12 ng/ml, less than 10 ng/ml, less than 8 ng/ml, less than 6 ng/ml, less than 4 ng/mL, less than 2 ng/ml, less than 1.5 ng/mL, less than 1.0 ng/mL, less than 0.5 ng/ml, less than 0.3 ng/mL, or less than 0.2 ng/mL.

A dry powder disclosed herein may be administered to achieve a certain steady state plasma concentration of the angiogenesis inhibitor (e.g., itraconazole). At steady state, the plasma concentration may be less than 25 ng/ml, less than 20 ng/ml, less than 15 ng/ml, less than 12 ng/ml, less than 10 ng/ml, less than 8 ng/ml, less than 6 ng/ml, less than 4 ng/ml, less than 2 ng/mL, less than 1.5 ng/ml, less than 1.0 ng/ml, less than 0.5 ng/ml, less than 0.3 ng/ml, or less than 0.2 ng/ml. Additionally, the steady state plasma concentration may be less than 40 ng/mL, less than 35 ng/mL, or less than 30 ng/mL.

Lung concentration of an angiogenesis inhibitor may be determined, for example, based on the concentration of the angiogenesis inhibitor in sputum or in lung tissue (e.g., lung tumor tissue). The lung concentration may be measured at the maximum concentration (i.e., the “Cₘₐₓ”) of the angiogenesis inhibitor (e.g., itraconazole) in the lung tissue or sputum, or at any point in time. The lung concentration may be measured at any point in the dosing cycle and may be calculated before or at steady state.

For example, a dry powder disclosed herein may be administered in one or more doses to achieve a lung concentration (sputum) of an angiogenesis inhibitor (e.g., itraconazole) of at least 100 ng/mL, such as about 500 ng/mL, about 800 ng/mL, about 1200 ng/mL, about 1600 ng/mL, about 2000 ng/mL, about 3000 ng/ml, about 4000 ng/ml, about 5000 ng/mL, about 6000 ng/ml, about 7000 ng/mL, about 8000 ng/ml, about 10,000 ng/ml, about 15,000 ng/mL, about 20,000 ng/mL, about 25,000 ng/mL, about 50,000 ng/mL, about 75,000 ng/mL, about 100,000 ng/mL, about 125,000 ng/mL, about 150,000 ng/mL, about 175,000 ng/mL, about 200,000 ng/mL, or more; or between about 500 ng/mL to 400,000 ng/mL, between about 500 ng/mL to 300,000 ng/mL, between about 500 ng/mL to 200,000 ng/ml, between about 500 ng/mL to 100,000 ng/mL, between about 500 ng/mL to 50,000 ng/mL, between about 500 ng/mL to 25,000 ng/mL, between about 500 ng/mL to 10,000 ng/mL, about 2000 ng/mL to 8000 ng/mL, or about 2000 ng/mL to 8100 ng/mL.

A dry powder disclosed herein may be administered in one or more doses to achieve a lung concentration (tissue, e.g., tumor tissue) of an angiogenesis inhibitor (e.g., itraconazole) of at least about 100 ng/g, such as about 500 ng/g, about 800 ng/g, about 1200 ng/g, about 1600 ng/g, about 2000 ng/g, about 3000 ng/g, about 4000 ng/g, about 5000 ng/g, about 6000 ng/g, about 7000 ng/g, about 8000 ng/g, about 9000 ng/g, about 10.000 ng/g, or more. In some embodiments, a dry powder disclosed herein may be administered in one or more doses to achieve a lung concentration (tissue, e.g., tumor tissue) of an angiogenesis inhibitor (e.g., itraconazole) of up to about 1.6 mg/g, up to about 1.4 mg/g, up to about 1.2 mg/g, or up to about 1.0 mg/g. In some embodiments, a dry powder disclosed herein may be administered in one or more doses to achieve a lung concentration (tissue, e.g., tumor tissue) of an angiogenesis inhibitor (e.g., itraconazole) of between about 100 ng/g and about 900,000 ng/g, e.g., between about 100 ng/g and about 800,000 ng/g, between about 100 ng/g and about 700,000 ng/g, between about 100 ng/g and about 600.000 ng/g, between about 100 ng/g and about 500,000 ng/g, between about 100 ng/g and about 400,000 ng/g, between about 100 ng/g and about 300,000 ng/g, between about 100 ng/g and about 200,000 ng/g, or between about 100 ng/g and about 100,000 ng/g.

In some embodiments, a dry powder disclosed herein may be administered in one or more doses to achieve a lung concentration (tissue, e.g., tumor tissue) of an angiogenesis inhibitor (e.g., itraconazole) of between about 100 ng/g and about 1.6 mg/g, e.g., about 500 ng/g, about 1000 ng/g, about 1500 ng/g, about 2000 ng/g, about 2500 ng/g, about 5000 ng/g, about 10,000 ng/g, about 15,000 ng/g, about 20,000 ng/g, about 25,000 ng/g, about 30,000 ng/g, about 40.000 ng/g, about 50.000 ng/g, about 60.000 ng/g, about 70.000 ng/g, about 80,000 ng/g, about 90,000 ng/g, about 100,000 ng/g, about 120,000 ng/g, about 140,000 ng/g, about 160,000 ng/g, about 180,000 ng/g, about 200,000 ng/g, about 250,000 ng/g, about 300,000 ng/g, about 350,000 ng/g, about 400,000 ng/g, about 450,000 ng/g, about 500,000 ng/g, about 600,000 ng/g, about 700,000 ng/g, about 800,000 ng/g, about 900,000 ng/g, about 1 mg/g, about 1.2 mg/g, about 1.4 mg/g, or about 1.6 mg/g.

Lung concentration of a metabolite of the angiogenesis inhibitor may be determined using similar methods. For example, a dry powder disclosed herein comprising itraconazole may be administered in one or more doses to achieve a lung concentration (sputum) of hydroxyitraconazole of at least 10 ng/mL, such as about 15 ng/mL, about 25 ng/mL, about 50 ng/mL, about 75 ng/mL, about 100 ng/mL, about 200 ng/mL, about 300 ng/mL, about 400 ng/mL, about 500 ng/mL, about 800 ng/mL, about 1200 ng/mL, about 1600 ng/mL, about 2000 ng/mL, about 3000 ng/mL, about 4000 ng/mL, about 5000 ng/mL, about 6000 ng/mL, about 7000 ng/mL, about 8000 ng/mL, or more; or between about 500 ng/mL to 8000 ng/mL, about 2000 ng/mL to 8000 ng/mL, or about 2000 ng/mL to 8100 ng/mL.

A dry powder disclosed herein comprising itraconazole may be administered in one or more doses to achieve a lung concentration (tissue, e.g., tumor tissue) of hydroxyitraconazole of at least about 10 ng/g, such as about 25 ng/g, about 50 ng/g, about 75 ng/g, about 100 ng/g, about 200 ng/g, about 300 ng/g, about 400 ng/g, about 500 ng/g, about 800 ng/g, about 1200 ng/g, about 1600 ng/g, about 2000 ng/g, about 3000 ng/g, about 4000 ng/g, about 5000 ng/g, about 6000 ng/g, about 7000 ng/g, about 8000 ng/g, about 9000 ng/g, about 10,000 ng/g or more.

A dry powder disclosed herein may be administered in one or more doses to achieve a steady state lung concentration of an angiogenesis inhibitor (e.g., itraconazole) in sputum of about 500 ng/mL, about 800 ng/mL, about 1200 ng/mL, about 1600 ng/mL, about 2000 ng/mL, about 3000 ng/mL, about 4000 ng/mL, about 5000 ng/mL, about 5000 ng/mL, about 6000 ng/mL, about 7000 ng/mL, about 8000 ng/mL, about 10,000 ng/mL, about 15,000 ng/mL, about 20,000 ng/mL, about 25,000 ng/mL, about 50,000 ng/mL, about 75,000 ng/mL, or more, e.g., between 2000 ng/mL to 100,000 ng/mL, between 2000 ng/mL to 50,000 ng/mL, between 2000 ng/mL to 10,000 ng/mL, between 2000 ng/mL to 8000 ng/mL, or between 2000 ng/mL to 8100 ng/mL.

A dry powder disclosed herein may be administered in one or more doses to achieve a steady state lung concentration of an angiogenesis inhibitor (e.g., itraconazole) in tissue (e.g., tumor tissue) of between about 100 ng/g and about 1.6 mg/g, e.g., about 500 ng/g, about 1000 ng/g, about 1500 ng/g, about 2000 ng/g, about 2500 ng/g, about 5000 ng/g, about 10,000 ng/g, about 15,000 ng/g, about 20,000 ng/g, about 25,000 ng/g, about 30,000 ng/g, about 40,000 ng/g, about 50,000 ng/g, about 60,000 ng/g, about 70,000 ng/g, about 80,000 ng/g, about 90,000 ng/g, about 100,000 ng/g, about 120,000 ng/g, about 140,000 ng/g, about 160,000 ng/g, about 180,000 ng/g, about 200,000 ng/g, about 250,000 ng/g, about 300,000 ng/g, about 350,000 ng/g, about 400,000 ng/g, about 450,000 ng/g, about 500,000 ng/g, about 600,000 ng/g, about 700,000 ng/g, about 800,000 ng/g, about 900,000 ng/g, about 1 mg/g, about 1.2 mg/g, about 1.4 mg/g, or about 1.6 mg/g.

A dry powder disclosed herein may be administered in one or more doses to achieve a steady state lung concentration of a metabolite of an angiogenesis inhibitor (e.g., hydroxyitraconazole) in sputum of about 500 ng/mL, about 800 ng/mL, about 1200 ng/mL, about 1600 ng/mL, about 2000 ng/mL, about 3000 ng/mL, about 4000 ng/mL, about 5000 ng/mL, about 5000 ng/mL, about 6000 ng/mL, about 7000 ng/mL, about 8000 ng/mL, between 2000 ng/mL to 8000 ng/mL, or about 2000 ng/mL to 8100 ng/mL. A dry powder disclosed herein may be administered in one or more doses to achieve a steady state lung concentration of a metabolite of an angiogenesis inhibitor (e.g., hydroxyitraconazole) in tissue (e.g., tumor tissue) of about 10 ng/g, such as about 25 ng/g, about 50 ng/g, about 75 ng/g, about 100 ng/g, about 200 ng/g, about 300 ng/g, about 400 ng/g, about 500 ng/g, about 800 ng/g, about 1200 ng/g, about 1600 ng/g, about 2000 ng/g, about 3000 ng/g, about 4000 ng/g, about 5000 ng/g, about 6000 ng/g, about 7000 ng/g, about 8000 ng/g, about 9000 ng/g, about 10,000 ng/g or more.

A dry powder disclosed herein may be administered once a day, twice a day, thrice a day, once every other day, or once every three days, once weekly, for approximately 7 days, 14 days, 21 days, 28 days, 1 month, 2 months, 3 months, 4 months, 5 months, 6 months, 1 year, or continuously. In some embodiments, the dry powder is dosed once a day until steady state is achieved, and then less frequently thereafter for up to six months. In some embodiments, one or more doses needed to achieve a therapeutic concentration of angiogenesis inhibitor (e.g., itraconazole) in the lung is administered daily until steady state is reached, followed by one or more doses, e.g., at a lower dose, or with less frequently administered doses. However, it will be understood that the total daily usage of the compounds or compositions of the present disclosure will be decided by the attending physician within the scope of sound medical judgment.

Various methods and assays for determining lung and plasma concentrations are known in the art and may be used to measure the lung and plasma concentrations during and after administration of the dry powders. For example, bioassays or HPLC may be used to measure the amount of angiogenesis inhibitor in the lung (e.g., using induced sputum, bronchial lavage, spontaneous sputum, biopsy), or the amount of angiogenesis inhibitor in a tumor (e.g., using a biopsy), after the subject has been administered the dry powder, e.g., for at least 7 days, at least 14 days, at least 21 days, or at least 28 days.

The dry powders and/or respirable dry particles can be administered to the respiratory tract of a subject in need thereof using any suitable method, such as instillation techniques, and/or an inhalation device, such as a dry powder inhaler (DPI) or metered dose inhaler (MDI). A number of DPIs are available, such as, the inhalers disclosed is U.S. Pat. Nos. 4,995,385 and 4,069,819, Spinhaler® (Fisons, Loughborough, U.K.), Rotahalers®, Diskhaler® and Diskus® (GlaxoSmithKline, Research Triangle Technology Park, North Carolina), FlowCaps® (Hovione, Loures, Portugal), Inhalators® (Boehringer-Ingelheim, Germany), Aerolizer® (Novartis, Switzerland), high-resistance, ultrahigh-resistance and low-resistance RS-01™ (a well-known type of unit-dose capsule-based dry powder inhaler produced by Plastiape, Italy, and described in U.S. Pat. No. 7,284,552, and US 2018/0369513) and others known to those skilled in the art.

The following scientific journal articles are incorporated by reference for their thorough overview of the following dry powder inhaler (DPI) configurations; 1) Single-dose Capsule DPI. 2) Multi-dose Blister DPI, and 3) Multi-dose Reservoir DPI. N. Islam, E. Gladki, “Dry powder inhalers (DPIs)—A review of device reliability and innovation”, International Journal of Pharmaceuticals, 360(2008):1-11. H. Chystyn, “Diskus Review”, International Journal of Clinical Practice, June 2007, 61, 6, 1022-1036. H. Steckel, B. Muller, “In vitro evaluation of dry powder inhalers I; drug deposition of commonly used devices”, International Journal of Pharmaceuticals, 154(1997):19-29. Some representative capsule-based DPI units are RS-01™ (Plastiape, Italy), Turbospin® (PH&T, Italy), Brezhaler® (Novartis, Switzerland), Aerolizer (Novartis, Switzerland), Podhaler® (Novartis, Switzerland). HandiHaler® (Boehringer Ingelheim, Germany), AIR® (Civitas, Massachusetts), Dose One® (Dose One, Maine), and Eclipse® (Rhone Poulenc Rorer). Some representative unit dose DPIs are Conix® (3M, Minnesota), Cricket® (Mannkind, California), Dreamboat® (Mannkind, California), Occoris® (Team Consulting, Cambridge, UK), Solis® (Sandoz), Trivair® (Trimel Biopharma, Canada), Twincaps® (Hovione, Loures, Portugal). Some representative blister-based DPI units are Diskus® (GlaxoSmithKline (GSK), UK), Diskhaler® (GSK), Taper Dry® (3M, Minnesota), Gemini® (GSK), Twincer® (University of Groningen, Netherlands), Aspirair® (Vectura, UK), Acu-Breathe® (Respirics, Minnesota, USA), Exubra® (Novartis, Switzerland), Gyrohaler® (Vectura, UK), Omnihaler® (Vectura, UK), Microdose® (Microdose Therapeutix, USA), Multihaler® (Cipla, India) Prohaler® (Aptar), Technohaler® (Vectura, UK), and Xcelovair® (Mylan, Pennsylvania). Some representative reservoir-based DPI units are Clickhaler® (Vectura), Next DPI® (Chiesi), Easyhaler® (Orion), Novolizer® (Meda), Pulmojet® (sanofi-aventis), Pulvinal® (Chiesi), Skyehaler® (Skyepharma), Duohaler® (Vectura), Taifun® (Akela), Flexhaler® (AstraZeneca, Sweden), Turbuhaler® (AstraZeneca, Sweden), and Twisthaler® (Merck), and others known to those skilled in the art.

Generally, inhalation devices (e.g., DPIs) are able to deliver a maximum amount of dry powder or dry particles in a single inhalation, which is related to the capacity of the blisters, capsules (e.g., size 000, 00, 0E, 0, 1, 2, 3 and 4, with respective volumetric capacities of 1.37 mL, 950 μL, 770 μL, 680 μL, 480 μL, 360 μL, 270 μL and 200 μL) or other means that contain the dry powders and/or respirable dry particles within the inhaler. Preferably, the blister has a volume of about 360 microliters or less, about 270 microliters or less, or more preferably, about 200 microliters or less, about 150 microliters or less, or about 100 microliters or less. Preferably, the capsule is a size 2 capsule, or a size 4 capsule. More preferably, the capsule is a size 3 capsule. Accordingly, delivery of a desired dose or effective amount may require two or more inhalations. Preferably, each dose that is administered to a subject in need thereof comprises an effective amount of respirable dry particles or dry powder and is administered using no more than about 4 inhalations. For example, each dose of dry powder or respirable dry particles can be administered in a single inhalation or 2, 3, or 4 inhalations. The dry powders and/or respirable dry particles are preferably administered in a single, breath-activated step using a passive DPI. When this type of device is used, the energy of the subject's inhalation both disperses the respirable dry particles and draws them into the respiratory tract.

Dry powders and/or respirable dry particles suitable for use in the methods of the invention can travel through the upper airways (i.e., the oropharynx and larynx), the lower airways, which include the trachea followed by bifurcations into the bronchi and bronchioli, and through the terminal bronchioli which in turn divide into respiratory bronchioli leading then to the ultimate respiratory zone, the alveoli or the deep lung. In one embodiment of the invention, most of the mass of respirable dry particles deposit in the deep lung. In another embodiment of the invention, delivery is primarily to the central airways. In another embodiment, delivery is to the upper airways. In a preferred embodiment, most of the mass of the respirable dry particles deposit in the conducting airways.

The dry powders and respirable dry particles described herein are intended to be inhaled as such, and the present disclosure excludes the use of the dry powder in making an extemporaneous dispersion. An extemporaneous dispersion is known by those skilled in the art as a preparation completed just before use, which means right before the administration of the drug to the patient.

### Dry Powders and Dry Particles

The present disclosure relates to methods of treatment comprising administering to a subject in need thereof a dry powder comprising respirable dry particles that comprise an angiogenesis inhibitor. The dry powder may also comprise a stabilizer and/or one or more excipients.

The angiogenesis inhibitor may be a compound that comprises one or more 1,2,4-triazole rings, such as itraconazole. In some embodiments, the angiogenesis inhibitor is an inhibitor of hedgehog signaling. In some embodiments, the angiogenesis inhibitor is a CYP51 inhibitor. In some embodiments, the dry powder comprises an agent that is commonly used as an antifungal agent, for example, itraconazole, fluconazole, fosfluconazole, voriconazole, posaconazole, albaconazole, efinaconazole, ravuconazole, fosravuconazole, isavuconazole, or a salt thereof. In preferred aspects, the dry powder comprises itraconazole (e.g., crystalline itraconazole).

Alternatively, the dry powder may comprise an angiogenesis inhibitor selected from the group consisting of bevacizumab (AVASTIN®), axitinib (INLYTA®), cabozantinib (COMETRIQ®, CABOMETYX®), everolimus (AFINITOR®), lenalidomide (REVLIMID®), lenvatinib mesylate (LENVIMA®), nintedanib (VARGATEF®, OFEV®), pazopanib (VOTRIENT®), ramucirumab (CYRAMZA®), regorafenib (STIVARGA®), sorafenib (NEXAVAR®), sunitinib (SUTENT®), thalidomide (SYNOVIR, THALOMID↔), vandetanib (CAPRELSA®), or ziv-aflibercept (ZALTRAP®). Alternatively, the dry powder comprises a compound disclosed in one of U.S. Pat. Nos. 8,653,083, 9,095,589, 9,346,791, 11,028,078; or International Applications WO 2013/155218, WO 2015/116947, or WO 2020/072830, each of which are incorporated herein by reference in their entireties. In preferred aspects, the angiogenesis inhibitor is itraconazole.

The crystallinity of the angiogenesis inhibitor (e.g., itraconazole), as well as the size of the angiogenesis inhibitor sub-particles, may be important for effective therapy and for reduced toxicity in the lung. For example, and without wishing to be bound by any particular theory, it is believed that smaller sub-particles of itraconazole in crystalline form will dissolve in the airway lining fluid more rapidly than larger particles of itraconazole in the same crystalline form—in part due to the larger amount of surface area. It is also believed that crystalline itraconazole will dissolve more slowly in the airway lining fluid than amorphous itraconazole. Accordingly, the dry powders described herein can be formulated using an angiogenesis inhibitor (e.g., itraconazole) in crystalline particulate form, that provide for a desired degree of crystallinity and sub-particle size, and can be tailored to achieve desired pharmacokinetic properties while avoiding unacceptable toxicity in the lungs.

The respirable dry particles may comprise an angiogenesis inhibitor (e.g., itraconazole) in an amount of about 1% to about 95% by weight (wt %). It is preferred that the respirable dry particle comprises an amount of angiogenesis inhibitor so that a therapeutically effective dose can be administered and maintained without the need to inhale large volumes of dry powder, and also without the need to inhale the dry powder too frequently, e.g., more than three time a day. For example, it is preferred that the respirable dry particles comprise about 1% to 95%, about 10% to 75%, about 15% to 75%, about 25% to 75%, about 30% to 70%, about 40% to 60%, about 50% to 95%, about 50% to 90%, about 50% to 70%, about 70% to 90%, about 60% to 80%, about 20%, about 30%, about 40%, about 50%, about 60%, or about 70% angiogenesis inhibitor (e.g., itraconazole) by weight (wt %). The respirable dry particles may comprise about 75%, about 80%, about 85%, about 90%, or about 95% angiogenesis inhibitor (e.g., itraconazole) by weight (wt %). In particular embodiments, the range of angiogenesis inhibitor in the respirable dry particles is about 40% to about 90%, about 55% to about 85%, about 55% to about 75%, or about 65% to about 85%, by weight (wt %). For example, the respirable dry particles may comprise about 40% by weight; about 41% by weight; about 42% by weight; about 43% by weight; about 44% by weight; about 45% by weight; about 46% by weight; about 47% by weight; about 48% by weight; about 49% by weight; about 50% by weight; about 51% by weight; about 52% by weight; about 53% by weight; about 54% by weight; about 55% by weight; about 56% by weight; about 57% by weight; about 58% by weight; about 59% by weight; or about 60% by weight of the angiogenesis inhibitor (e.g., itraconazole). The amount of angiogenesis inhibitor present in the respirable dry particles by weight may also be referred to as the “drug load.”

The angiogenesis inhibitor (e.g., itraconazole) can be present in the respirable dry particles in crystalline particulate form (e.g., nano-crystalline). More specifically, in the form of a sub-particle that is about 50 nm to about 5,000 nm (Dv50), preferably, with the angiogenesis inhibitor being at least 50% crystalline. For example, for any desired load of the angiogenesis inhibitor (sometimes referred to as “drug load”), the sub-particle size can be about 100 nm, about 300 nm, about 1500 nm, about 80 nm to about 300 nm, about 80 nm to about 250 nm, about 80 nm to about 200 nm, about 100 nm to about 150 nm, about 1200 nm to about 1500 nm, about 1500 nm to about 1750 nm, about 1200 nm to about 1400 nm, or about 1200 nm to about 1350 nm (Dv50). In particular embodiments, the sub-particle is between about 50 nm to about 2500 nm, between about 80 and 1750 nm, between about 50 nm and 1000 nm, between about 50 nm and 800 nm, between about 50 nm and 600 nm, between about 50 nm and 500 nm, between about 50 nm and 400 nm, between about 50 nm and 300 nm, between about 50 nm and 200 nm, or between about 100 nm and 300 nm. In addition, for any desired drug load and sub-particle size, the degree of angiogenesis inhibitor crystallinity can be at least about 50%, at least about 60%, at least about 70%, at least about 80%, at least about 90%, at least about 95%, or about 100% crystalline. Preferably, the angiogenesis inhibitor is about 100% crystalline. In some embodiments, the dry powder administered comprises homogenous respirable dry particles that comprise an angiogenesis inhibitor (e.g., itraconazole) that is at least 50% crystalline, e.g., 55% crystalline, 60% crystalline, 65% crystalline, 70% crystalline, 75% crystalline, 80% crystalline, 85% crystalline, 90% crystalline, 95% crystalline, 96% crystalline, 97% crystalline, 98% crystalline, 99% crystalline, or more than 99% crystalline

The angiogenesis inhibitor (e.g., itraconazole) in crystalline particulate form can be prepared in any desired sub-particle size using a suitable method, including a stabilizer if desired, such as by wet milling, jet milling or other suitable method.

The respirable dry particles also include a stabilizer. The stabilizer helps maintain the desired size of the angiogenesis inhibitor (e.g., itraconazole) in crystalline particulate form during wet milling, in spray drying feedstock, and aids in wetting and dispersing and maintaining the physical stability of the angiogenesis inhibitor crystalline particulate suspension. It is preferred to use as little stabilizer as is needed to achieve the aforementioned benefits. The amount of stabilizer is typically in a fixed ratio to the amount of angiogenesis inhibitor present in the dry particle and can range from about 1:1 (angiogenesis inhibitor:stabilizer (wt:wt)) to about 50:1 (wt:wt), with ≥ (greater than or equal to) 10:1 being preferred. For example, the ratio of angiogenesis inhibitor:stabilizer (wt:wt) in the dry particles can be ≥ (greater than or equal to) 10:1, about 10:1, about 20:1, about 1:1 to about 50:1, about 10:1 to about 15:1, or about 10:1 to about 20:1. In particular embodiments, the ratio is about 5:1 to about 20:1, about 7:1 to about 15:1, or about 9:1 to about 11:1. Alternatively, the ratio of angiogenesis inhibitor:stabilizer (wt:wt) in the dry particles may be greater than 10:1 to 25:1, 11:1 to 35:1, 10.5:1 to 14.5:1, 11:1 to 31:1, greater than 12:1, 11:1 to 15:1, 11.5:1 to 14:1, 13:1 to 16:1, or 15:1 to 19.5:1, 19:1 to 25:1, 20.5:1 to 23:1, 22:1 to 32:1. Alternatively, the ratio of angiogenesis inhibitor:stabilizer (wt:wt) in the dry particles can be greater than or equal to 11.5:1, greater than or equal to 12:1, greater than or equal to 14:1, greater than or equal to 15:1, greater than or equal to 16:1, greater than or equal to 17:1, greater than or equal to 18:1, greater than or equal to 19:1; about 11:1, about 12:1, about 13:1, about 14:1, about 15:1, about 18:1, about 19.5:1, or about 22:1.

In addition, the amount of stabilizer that is present in the dry particles can be in a range of about 0.05% to about 45% by weight (wt %). In particular embodiments, the range is about 1% to about 15%, about 4% to about 10%, or about 5% to about 8% by weight (wt %). It is generally preferred that the respirable dry particles comprise less than about 10% stabilizer by weight (wt %), such as 9 wt % or less, 8 wt % or less, 7 wt % or less, 5 wt % or less, 4 wt % or less, 3 wt % or less, 2 wt % or less, or 1 wt % or less. Alternatively, the respirable dry particles comprise about 5 wt %, about 6 wt %, about 7 wt %, about 7.5 wt %, about 8 wt %, or about 10% stabilizer. A particularly preferred stabilizer for use in the dry powders described herein is polysorbate 80. Another preferred stabilizer is oleic acid, or a salt thereof. In contrast to conventional dry powders which use surfactant to prevent the onset of crystallization in the dry powder, the surfactant in the present invention is added to stabilize a colloidal suspension of the crystalline compound (e.g., angiogenesis inhibitor) in an anti-solvent.

In some embodiments, the dry powder administered comprises homogenous respirable dry particles that comprise an angiogenesis inhibitor (e.g., itraconazole) and a stabilizer (e.g., polysorbate 80, or oleic acid or a salt thereof), wherein the ratio of angiogenesis inhibitor:stabilizer (wt:wt) is from about 1:1 to 50:1; greater than or equal to 10:1; about 10:1; about 20:1; about 5:1 to about 20:1; about 7:1 to about 15:1; or about 9:1 to about 11:1. In some embodiments, the stabilizer is present in an amount of about 0.05% to about 45% by weight; about 4% to about 10% by weight

The respirable dry particles also include any suitable and desired amount of one or more excipients. The dry particles can comprise a total excipient content of about 10 wt % to about 99 wt %, with about 25 wt % to about 85 wt %, or about 40 wt % to about 55 wt % being more typical. The dry particles can comprise a total excipient content of about 1 wt %, about 2 wt %, about 4 wt %, about 6 wt %, about 8 wt %, or less than about 10 wt %. In particular embodiments, the range is about 5% to about 50%, about 15% to about 50%, about 25% to about 50%, about 5% to about 40%, about 5% to about 30%, about 5% to about 20%, or about 5% to about 15%. In other embodiments, the range of excipient is about 1% to about 9%, about 2% to about 9%, about 3% to about 9%, about 4% to about 9%, about 5% to about 9%, about 1% to about 8%, about 2% to about 8%, about 3% to about 8%, about 4% to about 8%, about 5% to about 8%, about 1% to about 7%, about 2% to about 7%, about 3% to about 7%, about 4% to about 7%, about 5% to about 7%, about 1% to about 6%, about 2% to about 6%, about 3% to about 6%, or about 1% to about 5%.

In some embodiments, the dry powder administered comprises one or more excipients that are present in an amount of about 10% to about 99% by weight, e.g., 10%, 15%, 20%, 25%, 30%, 35%, 40%, 45%, 50%, 55%, 60%, 65%, 70%, 75%, 80%, 85%, 90%, 95%, 96%, 97%, 98% or 99%. In some embodiments, the one or more excipients are present in an amount of about 5% to about 50% by weight.

Many excipients are well-known in the art and can be included in the dry powders and dry particles described herein. Pharmaceutically acceptable excipients that are particularly preferred for the dry powders and dry particles described herein include monovalent and divalent metal cation salts, carbohydrates, sugar alcohols and amino acids.

Suitable monovalent metal cation salts, include, for example, sodium salts and potassium salts. Suitable sodium salts that can be present in the respirable dry particles of the invention include, for example, sodium chloride, sodium citrate, sodium sulfate, sodium lactate, sodium acetate, sodium bicarbonate, sodium carbonate, sodium stearate, sodium ascorbate, sodium benzoate, sodium biphosphate, sodium phosphate, sodium bisulfite, sodium borate, sodium gluconate, sodium metasilicate and the like.

Suitable potassium salts include, for example, potassium chloride, potassium bromide, potassium iodide, potassium bicarbonate, potassium nitrite, potassium persulfate, potassium sulfite, potassium bisulfite, potassium phosphate, potassium acetate, potassium citrate, potassium glutamate, dipotassium guanylate, potassium gluconate, potassium malate, potassium ascorbate, potassium sorbate, potassium succinate, potassium sodium tartrate, and any combination thereof.

Suitable divalent metal cation salts, include magnesium salts and calcium salts. Suitable magnesium salts include, for example, magnesium lactate, magnesium fluoride, magnesium chloride, magnesium bromide, magnesium iodide, magnesium phosphate, magnesium sulfate, magnesium sulfite, magnesium carbonate, magnesium oxide, magnesium nitrate, magnesium borate, magnesium acetate, magnesium citrate, magnesium gluconate, magnesium maleate, magnesium succinate, magnesium malate, magnesium taurate, magnesium orotate, magnesium glycinate, magnesium naphthenate, magnesium acetylacetonate, magnesium formate, magnesium hydroxide, magnesium stearate, magnesium hexafluorsilicate, magnesium salicylate or any combination thereof.

Suitable calcium salts include, for example, calcium chloride, calcium sulfate, calcium lactate, calcium citrate, calcium carbonate, calcium acetate, calcium phosphate, calcium alginate, calcium stearate, calcium sorbate, calcium gluconate and the like.

A preferred sodium salt is sodium sulfate. A preferred sodium salt is sodium chloride. A preferred sodium salt is sodium citrate. A preferred magnesium salt is magnesium lactate.

Carbohydrate excipients that are useful in this regard include the mono- and polysaccharides, sugar alcohols, dextrans, dextrins, and cyclodextrins, amongst others. Representative monosaccharides include dextrose (anhydrous and the monohydrate; also referred to as glucose and glucose monohydrate), galactose, D-mannose, sorbose and the like. Representative disaccharides include lactose, maltose, sucrose, trehalose and the like. Representative trisaccharides include raffinose and the like. Other carbohydrate excipients including dextran, maltodextrin and cyclodextrins, such as 2-hydroxypropyl-beta-cyclodextrin can be used as desired. Representative sugar alcohols include mannitol, sorbitol and the like. A preferred sugar alcohol is mannitol.

Suitable amino acid excipients include any of the naturally occurring amino acids that form a powder under standard pharmaceutical processing techniques and include the non-polar (hydrophobic) amino acids and polar (uncharged, positively charged and negatively charged) amino acids, such amino acids are of pharmaceutical grade and are generally regarded as safe (GRAS) by the U.S. Food and Drug Administration. Representative examples of non-polar amino acids include alanine, isoleucine, leucine, methionine, phenylalanine, proline, tryptophan and valine. Representative examples of polar, uncharged amino acids include cysteine, glycine, glutamine, serine, threonine, and tyrosine. Representative examples of polar, positively charged amino acids include arginine, histidine and lysine. Representative examples of negatively charged amino acids include aspartic acid and glutamic acid. A preferred amino acid is leucine.

In one aspect, the respirable dry particles comprise leucine as one of the one or more excipients in an amount of about 1% to about 9%, about 2% to about 9%, about 3% to about 9%, about 4% to about 9%, about 5% to about 9%, about 1% to about 8%, about 2% to about 8%, about 3% to about 8%, about 4% to about 8%, about 5% to about 8%, about 1% to about 7%, about 2% to about 7%, about 3% to about 7%, about 4% to about 7%, about 5% to about 7%, about 1% to about 6%, about 2% to about 6%, about 3% to about 6%, about 1% to about 5%, about 1%, about 2%, about 3%, about 4%, about 5%, about 6%, about 7%, about 9%, or about 10%. On another aspect, the respirable dry particles comprise leucine as one of the one or more excipients in an amount of 10% or greater.

Without wishing to be bound by theory, it is believed that combining the angiogenesis inhibitor in a dry powder with an amino acid, such as leucine, and optionally one or more excipients, such as a monovalent metal cation (e.g., a sodium salt, e.g., sodium chloride or sodium sulfate), and optionally a stabilizer (e.g., polysorbate 80, oleic acid, or a salt thereof) can provide optimal dissolution rates for obtaining effective therapeutic levels of the angiogenesis inhibitor in the lungs without unacceptable toxicity. Additionally, maintaining a relatively high drug load (e.g., 40%, 50%, or more) of the angiogenesis inhibitor may prevent rapid dissolution of the dry powder in the lungs. For example, the dry powders disclosed herein may dissolve in the lungs more slowly, compared to a formulation combining relatively low amounts of itraconazole (e.g., less than 40 wt %) with a hydrophilic excipient such as mannitol.

The dissolution of dry powders used in methods disclosed herein may be measured in terms of the dissolution half-life. In some embodiments, the dry powders used in a method disclosed herein have a dissolution half-life that is at least about 2 minutes, e.g., between about 2 minutes and about 60 minutes, between about 2 minutes and about 40 minutes, between about 2 minutes and about 30 minutes, between about 3 minutes and about 25 minutes, between about 4 minutes and about 20 minutes, between about 4 minutes and about 18 minutes, between about 2 and about 10 minutes, between about 10 and about 20 minutes, between about 4 and about 5 minutes, between about 5 and about 6 minutes, between about 7 and about 8 minutes, e.g., about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 16 minutes, about 18 minutes, about 20 minutes, about 30 minutes, or about 40 minutes. In some embodiments, the dissolution half-life is about 4.1 minutes, about 4.2 minutes, about 4.3 minutes, or about 4.4 minutes. In some embodiments the dissolution half-life is about 7.2 minutes, about 7.3 minutes, about 7.4 minutes, or about 7.5 minutes. In some embodiments, the dissolution half-life is about 16.6 minutes, about 16.7 minutes, about 16.8 minutes, or about 16.9 minutes. In some embodiments the dissolution half-life is between about 4.13 minutes and about 16.84 minutes.

The dry particles described herein comprise an angiogenesis inhibitor (e.g., itraconazole, such as itraconazole in crystalline particulate form), and optionally a stabilizer and/or one or more excipients. In some aspects, the dry particles comprise a first excipient that is a monovalent or divalent metal cation salt, and a second excipient that is an amino acid, carbohydrate or sugar alcohol. For example, the first excipient can be a sodium salt or a magnesium salt, and the second excipient can be an amino acid (such as leucine). In more particular examples, the first excipient can be sodium sulfate, sodium chloride or magnesium lactate, and the second excipient can be leucine. Even more particularly, the first excipient can be sodium sulfate and the second excipient can be leucine. In another example, the first excipient can be a sodium salt or a magnesium salt, and the second excipient can be a sugar alcohol (such as mannitol). In more particular examples, the first excipient can be sodium sulfate, sodium chloride or magnesium lactate, and the second excipient can be mannitol. In another example, the first excipient can be a sodium salt or a magnesium salt, and the second excipient can be a carbohydrate (such as maltodextrin). In other examples, the dry particles include an angiogenesis inhibitor in crystalline particulate form, a stabilizer and one excipient, for example a sodium salt, a magnesium salt or an amino acid (e.g. leucine).

In one aspect, the dry powder comprises respirable dry particles comprising 1) an angiogenesis inhibitor in crystalline particulate form. 2) a stabilizer, and 3) one or more excipients.

In one preferred aspect, the dry powder comprises respirable dry particles comprising 1) itraconazole in crystalline particulate form, 2) a stabilizer, and 3) one or more excipients.

In one aspect, the dry powder comprises respirable dry particles comprising: (i) about 50% to about 80% of an angiogenesis inhibitor (e.g., itraconazole) in crystalline particulate form, about 4% to about 40% of a stabilizer, and about 1% to about 9% of one or more excipients; (ii) about 45% to about 85% of an angiogenesis inhibitor (e.g., itraconazole) in crystalline particulate form, about 3% to about 15% of a stabilizer, about 3% to about 40% sodium salt, and about 1% to about 9% of one or more amino acids; (iii) about 45% to about 85% of an angiogenesis inhibitor (e.g., itraconazole) in crystalline particulate form, about 3% to about 15% of a stabilizer, about 3% to about 50% sodium sulfate, and about 1% to 9% of leucine; (iv) about 45% to about 85% of an angiogenesis inhibitor (e.g., itraconazole) in crystalline particulate form, about 3% to about 15% of a stabilizer, about 3% to about 50% sodium salt, and about 1% to about 8% of one or more amino acids; or (v) about 45% to about 85% of an angiogenesis inhibitor (e.g., itraconazole) in crystalline particulate form, about 3% to about 15% of a stabilizer, about 3% to about 40% sodium sulfate, and about 1% to about 8% of leucine; where all percentages are weight percentages, and all formulations add up to 100% on a dry basis.

In a particularly preferred aspect, the dry powder comprises respirable dry particles comprising 1) itraconazole in crystalline particulate form, 2) a stabilizer, and 3) one or more excipients. In this particularly preferred aspect, the dry powder does not comprise lactose. Specific formulations of this particularly preferred embodiment are below. In Tables 1 and 1A below, these examples are further specified for itraconazole in crystalline particulate form at specific itraconazole crystalline sizes, also referred to as itraconazole subparticles.

In one aspect, the dry powder comprises 50% itraconazole, 35% sodium sulfate, 10% leucine, and 5% polysorbate 80.

In one aspect, the dry powder comprises 50% itraconazole, 37% sodium sulfate, 8% leucine, and 5% polysorbate 80.

In another aspect, the dry powder comprises 60% itraconazole, 26% sodium sulfate, 8% leucine, and 6% polysorbate 80.

In another aspect, the dry powder comprises 70% itraconazole, 15% sodium, 8% leucine, and 7% polysorbate 80.

In another aspect, the dry powder comprises 75% itraconazole, 9.5% sodium sulfate, 8% leucine, and 7.5% polysorbate 80.

In another aspect, the dry powder comprises 80% itraconazole, 4% sodium sulfate, 8% leucine, and 8% polysorbate 80.

In another aspect, the dry powder comprises 80% itraconazole, 10% sodium sulfate, 2% leucine, and 8% polysorbate 80.

In another aspect, the dry powder comprises 80% itraconazole, 11% sodium sulfate, 1% leucine, and 8% polysorbate 80.

In one aspect, the dry powder comprises 50% itraconazole, 35% sodium chloride, 10% leucine, and 5% polysorbate 80.

In one aspect, the dry powder comprises 50% itraconazole, 37% sodium chloride, 8% leucine, and 5% polysorbate 80.

In another aspect, the dry powder comprises 60% Itraconazole, 26% sodium chloride, 8% leucine, and 6% polysorbate 80.

In another aspect, the dry powder comprises 70% itraconazole, 15% sodium chloride, 8% leucine, and 7% polysorbate 80.

In another aspect, the dry powder comprises 75% itraconazole, 9.5% sodium chloride, 8% leucine, and 7.5% polysorbate 80.

In another aspect, the dry powder comprises 80% itraconazole, 4% sodium chloride, 8% leucine, and 8% polysorbate 80.

In another aspect, the dry powder comprises 80% itraconazole, 10% sodium chloride, 2% leucine, and 8% polysorbate 80.

In another aspect, the dry powder comprises 80% itraconazole, 11% sodium chloride, 1% leucine, and 8% polysorbate 80.

The dry powders and/or respirable dry particles are preferably small, mass dense, and dispersible. To measure volumetric median geometric diameter (VMGD), a laser diffraction system may be used, e.g., a Spraytec system (particle size analysis instrument, Malvern Instruments) and a HELOS/RODOS system (laser diffraction sensor with dry dispensing unit, Sympatec GmbH). The respirable dry particles have a VMGD as measured by laser diffraction at the dispersion pressure setting (also called regulator pressure) of 1.0 bar at a maximum orifice ring pressure using a HELOS/RODOS system of about 10 microns or less, about 5 microns or less, about 4 μm or less, about 3 μm or less, about 1 μm to about 5 μm, about 1 μm to about 4 μm, about 1.5 μm to about 3.5 μm, about 2 μm to about 5 μm, about 2 μm to about 4 μm, or about 2 μm to about 3 μm. Preferably, the VMGD is about 5 microns or less or about 4 μm or less. In one aspect, the dry powders and/or respirable dry particles have a minimum VMGD of about 0.5 microns or about 1.0 micron.

The dry powders and/or respirable dry particles preferably have 1 bar/4 bar dispersibility ratio and/or 0.5 bar/4 bar dispersibility ratio of less than about 2.0 (e.g., about 0.9 to less than about 2), about 1.7 or less (e.g., about 0.9 to about 1.7) about 1.5 or less (e.g., about 0.9 to about 1.5), about 1.4 or less (e.g., about 0.9 to about 1.4), or about 1.3 or less (e.g., about 0.9 to about 1.3), and preferably have a 1 bar/4 bar and/or a 0.5 bar/4 bar of about 1.5 or less (e.g., about 1.0 to about 1.5), and/or about 1.4 or less (e.g., about 1.0 to about 1.4).

The dry powders and/or respirable dry particles preferably have a tap density of at least about 0.2 g/cm³, of at least about 0.25 g/cm³, a tap density of at least about 0.3 g/cm³, of at least about 0.35 g/cm³, a tap density of at least 0.4 g/cm³. For example, the dry powders and/or respirable dry particles have a tap density of greater than 0.4 g/cm³ (e.g., greater than 0.4 g/cm³ to about 1.2 g/cm³), a tap density of at least about 0.45 g/cm³ (e.g., about 0.45 g/cm³ to about 1.2 g/cm³), at least about 0.5 g/cm³ (e.g., about 0.5 g/cm³ to about 1.2 g/cm³), at least about 0.55 g/cm³ (e.g., about 0.55 g/cm³ to about 1.2 g/cm³), at least about 0.6 g/cm³ (e.g., about 0.6 g/cm³ to about 1.2 g/cm³) or at least about 0.6 g/cm³ to about 1.0 g/cm³. Alternatively, the dry powders and/or respirable dry particles preferably have a tap density of about 0.01 g/cm³ to about 0.5 g/cm³, about 0.05 g/cm³ to about 0.5 g/cm³, about 0.1 g/cm³ to about 0.5 g/cm³, about 0.1 g/cm³ to about 0.4 g/cm³, or about 0.1 g/cm³ to about 0.4 g/cm³. Alternatively, the dry powders and/or respirable dry particles have a tap density of about 0.15 g/cm³ to about 1.0 g/cm³. Alternatively, the dry powders and/or respirable dry particles have a tap density of about 0.2 g/cm³ to about 0.8 g/cm³.

The dry powders and/or respirable dry particles have a bulk density of at least about 0.1 g/cm³, or at least about 0.8 g/cm³. For example, the dry powders and/or respirable dry particles have a bulk density of about 0.1 g/cm³ to about 0.6 g/cm³, about 0.2 g/cm³ to about 0.7 g/cm³, about 0.3 g/cm³ to about 0.8 g/cm³.

The respirable dry particles, and the dry powders when the dry powders are respirable dry powders, preferably have an MMAD of less than 10 microns, preferably an MMAD of about 5 microns or less, or about 4 microns or less. In one aspect, the respirable dry powders and/or respirable dry particles preferably have a minimum MMAD of about 0.5 microns, or about 1.0 micron. In one aspect, the respirable dry powders and/or respirable dry particles preferably have a minimum MMAD of about 2.0 microns, about 3.0 microns, or about 4.0 microns.

The dry powders and/or respirable dry particles preferably have a FPF of less than about 5.6 microns (FPF<5.6 μm) of the total dose of at least about 35%, preferably at least about 45%, at least about 60%, between about 45% to about 80%, or between about 60% and about 80%.

The dry powders and/or respirable dry particles preferably have a FPF of less than about 3.4 microns (FPF<3.4 μm) of the total dose of at least about 20%, preferably at least about 25%, at least about 30%, at least about 40%, between about 25% and about 60%, or between about 40% and about 60%.

The dry powders and/or respirable dry particles preferably have a total water and/or solvent content of up to about 15% by weight, up to about 10% by weight, up to about 5% by weight, up to about 1%, or between about 0.01% and about 1%, or may be substantially free of water or other solvent.

The dry powders and/or respirable dry particles preferably may be administered with low inhalation energy. In order to relate the dispersion of powder at different inhalation flow rates, volumes, and from inhalers of different resistances, the energy required to perform the inhalation maneuver may be calculated. Inhalation energy can be calculated from the equation E=R²Q²V where E is the inhalation energy in Joules, R is the inhaler resistance in kPa^{1/2}/LPM, Q is the steady flow rate in L/min and V is the inhaled air volume in L.

Healthy adult populations are predicted to be able to achieve inhalation energies ranging from 2.9 Joules for comfortable inhalations to 22 Joules for maximum inhalations by using values of peak inspiratory flow rate (PIFR) measured by Clarke et al. (Journal of Aerosol Med, 6(2), p.99-110, 1993) for the flow rate Q from two inhaler resistances of 0.02 and 0.055 kPa^{1/2}/LPM, with an inhalation volume of 2 L based on both FDA guidance documents for dry powder inhalers and on the work of Tiddens et al. (Journal of Aerosol Med, 19(4), p.456-465, 2006) who found adults averaging 2.2 L inhaled volume through a variety of DPIs.

Mild, moderate and severe adult COPD patients are predicted to be able to achieve maximum inhalation energies of 5.1 to 21 Joules, 5.2 to 19 Joules, and 2.3 to 18 Joules respectively. This is again based on using measured PIFR values for the flow rate Q in the equation for inhalation energy. The PIFR achievable for each group is a function of the inhaler resistance that is being inhaled through. The work of Broeders et al. (Eur Respir J, 18, p.780-783, 2001) was used to predict maximum and minimum achievable PIFR through two dry powder inhalers of resistances 0.021 and 0.032 kPa^{1/2}/LPM for each.

Similarly, adult asthmatic patients are predicted to be able to achieve maximum inhalation energies of 7.4 to 21 Joules based on the same assumptions as the COPD population and PIFR data from Broeders et al.

Healthy adults and children, for example, are capable of providing sufficient inhalation energy to disperse a dry powder of the present disclosure, e.g., from a suitable inhalation device (e.g., dry powder inhaler). It is also expected that most cancer patients, e.g., patients with a lung cancer such as NSCLC, are capable of providing sufficient inhalation energy to disperse a dry powder of the present disclosure, e.g., from a suitable inhalation device.

The dry powders and/or respirable dry particles useful in a method disclosed herein are preferably characterized by a high emitted dose, such as a CEPM of at least 75%, at least 80%, at least 85%, at least 90%, at least 95%, from a passive dry powder inhaler subject to a total inhalation energy of about 5 Joules, about 3.5 Joules, about 2.4 Joules, about 2 Joules, about 1 Joule, about 0.8 Joules, about 0.5 Joules, or about 0.3 Joules is applied to the dry powder inhaler. The receptacle holding the dry powders and/or respirable dry particles may comprise about 5 mg, about 7.5 mg, about 10 mg, about 15 mg, about 20 mg, or about 30 mg. In one aspect, the dry powders and/or respirable dry particles are characterized by a CEPM of 80% or greater and a VMGD of 5 microns or less when emitted from a passive dry powder inhaler having a resistance of about 0.036 sqrt (kPa)/liters per minute under the following conditions; an air flow rate of 30 LPM, run for 3 seconds using a size 3 capsule that comprises a total mass of 10 mg. In another aspect, the dry powders and/or respirable dry particles are characterized by a CEPM of 80% or greater and a VMGD of 5 microns or less when emitted from a passive dry powder inhaler having a resistance of about 0.036 sqrt (kPa)/liters per minute under the following conditions; an air flow rate of 20 LPM, run for 3 seconds using a size 3 capsule that comprises a total mass of 10 mg. In a further aspect, the dry powders and/or respirable dry particles are characterized by a CEPM of 80% or greater and a VMGD of 5 microns or less when emitted from a passive dry powder inhaler having a resistance of about 0.036 sqrt (kPa)/liters per minute under the following conditions; an air flow rate of 15 LPM, run for 4 seconds using a size 3 capsule that comprises a total mass of 10 mg.

The dry powder can fill the unit dose container, or the unit dose container can be at least 2% full, at least 5% full, at least 10% full, at least 20% full, at least 30% full, at least 40% full, at least 50% full, at least 60% full, at least 70% full, at least 80% full, or at least 90% full. The unit dose container can be a capsule (e.g., size 000, 00, 0E, 0, 1, 2, 3, and 4, with respective volumetric capacities of 1.37 mL, 950 μL, 770 μL, 680 μL, 480 μL, 360 μL, 270 μL, and 200 μL). The capsule can be at least about 2% full, at least about 5% full, at least about 10% full, at least about 20% full, at least about 30% full, at least about 40% full, or at least about 50% full. The unit dose container can be a blister. The blister can be packaged as a single blister or as part of a set of blisters, for example, 7 blisters, 14 blisters, 28 blisters or 30 blisters. The one or more blister can be preferably at least 30% full, at least 50% full or at least 70% full.

An advantage of the dry powders disclosed herein is that they disperse well across a wide range of flow rates and are relatively flowrate independent. The dry powders and/or respirable dry particles enable the use of a simple, passive DPI for a wide patient population.

In particular aspects, the dry powders and/or respirable dry particles that comprise an angiogenesis inhibitor in crystalline particulate form also referred to as angiogenesis inhibitor crystalline sub-particles (e.g., sub-particle size of about 80 nm to about 1750 nm, such as about 60 nm to about 175 nm, about 150 nm to about 400 nm or about 1200 nm to about 1750 nm), a stabilizer, and optionally one or more excipients. Particular dry powders and respirable dry particles have the following formulations shown in Table 1.

The dry powders and/or respirable dry particles useful in a method described herein are preferably characterized by; 1) a VMGD at 1 bar as measured using a HELOS/RODOS system of about 10 microns or less, preferably about 5 microns or less; 2) a 1 bar/4 bar dispersibility ratio and/or a 0.5 bar/4 bar dispersibility ratio of about 1.5 or less, about 1.4 or less or about 1.3 or less; 3) a MMAD of about 10 microns or less, preferably about 5 microns or less; 4) a FPF<5.6 μm of the total dose of at least about 45% or at least about 60%; and/or 5) a FPF<3.4 μm of the total dose of at least about 25% or at least about 40%. If desired, the dry powders and/or respirable dry particles are further characterized by a tap density of about 0.2 g/cm³ or greater, about 0.3 g/cm³ or greater, about 0.4 g/cm³ or greater, greater than 0.4 g/cm³, about 0.45 g/cm³ or greater or about 0.5 g/cm³ or greater.

Exemplary dry powders that can be used in a method disclosed herein are provided below in Table 1.

TABLE 1
Exemplary dry powders.

| | Angiogenesis | | | Angiogenesis inhibitor subparticle | |
|---|---|---|---|---|---|
| | Inhibitor | Excipients | Stabilizer | size (left) and range (right) | |
| Formulation | (wt %) | (wt %) | (wt %) | (Dv50, nm) | |
| A (I) | Itraconazole | Sodium sulfate 39% | PS80 2% | 124 | 60-175 |
| | 20% | Mannitol 39% | | | |
| B (II) | Itraconazole | Sodium sulfate | PS80 5% | 124 | 60-175 |
| | 50% | 22.5% | | | |
| | | Mannitol 22.5% | | | |
| C (III) | Itraconazole | Sodium chloride | PS80 2% | 124 | 60-175 |
| | 20% | 62.4% | | | |
| | | Leucine 15.6% | | | |
| D (IV) | Itraconazole | Sodium chloride | PS80 5% | 124 | 60-175 |
| | 50% | 36% | | | |
| | | Leucine 9% | | | |
| E (V) | Itraconazole | Magnesium lactate | PS80 2% | 124 | 60-175 |
| | 20% | 66.3% | | | |
| | | Leucine 11.7% | | | |
| F (VI) | Itraconazole | Magnesium lactate | PS80 5% | 124 | 60-175 |
| | 50% | 38.25% | | | |
| | | Leucine 6.75% | | | |
| G (VII) | Itraconazole | Sodium sulfate | Oleic | 120 | 60-175 |
| | 50% | 33.25% | acid 2.5% | | |
| | | Leucine 14.25% | | | |
| H (VIII) | Itraconazole | Sodium sulfate | Oleic | 120 | 60-175 |
| | 70% | 13.25% | acid 3.5% | | |
| | | Leucine 13.25% | | | |
| I | Itraconazole | Magnesium lactate | Oleic | 120 | 60-175 |
| | 50% | 33.25% | acid 2.5% | | |
| | | Leucine 14.25% | | | |
| J | Itraconazole | Magnesium lactate | Oleic | 120 | 60-175 |
| | 70% | 13.25% | acid 3.5% | | |
| | | Leucine 13.25% | | | |
| K (XI) | Itraconazole | Sodium sulfate 35% | Oleic | 126 | 60-175 |
| | 50% | Leucine 12. 5% | acid 2.5% | | |
| L (XII) | Itraconazole | Sodium sulfate 35% | PS80 5% | 132 | 60-175 |
| | 50% | Leucine 10% | | | |
| M (XIII) | Itraconazole | Sodium sulfate 35% | PS80 5% | 198 | 150-250 |
| | 50% | Leucine 10% | | | |
| N (XIV) | Itraconazole | Sodium sulfate 35% | PS80 5% | 258 | 200-325 |
| | 50% | Leucine 10% | | | |
| O (XV) | Itraconazole | Sodium sulfate 35% | PS80 5% | 1600 | 1200-1500 |
| | 50% | Leucine 10% | | | |
| P (XVI) | Itraconazole | Sodium sulfate 35% | PS80 <5% | 1510 | 1200-1500 |
| | 50% | Leucine 10% | | | |
| S (XIX) | Itraconazole | Sodium sulfate 35% | N/A | N/A | N/A |
| | 50% | Leucine 15% | | | Itraconazole in |
| | | | | | amorphous |
| | | | | | form |
| XX | Itraconazole | Sodium sulfate 35% | N/A | N/A | N/A |
| | 50% | Leucine 15% | | | Itraconazole in |
| | | | | | amorphous |
| | | | | | form |
| XXI | Itraconazole | Sodium sulfate 35%, | PS80 5% | 130 | 60-175 |
| | 50% | Leucine 10% | | | |
| XXII | Itraconazole | Sodium sulfate 35%, | Oleic | 115 | 60-175 |
| | 50% | Leucine 11.57% | acid | | |
| | | | 3.43% | | |
| XXIII | Itraconazole | Sodium sulfate 35%, | PS80 | 1640 | 1200-1650 |
| | 50% | Leucine 13.75% | 1.25% | | |
| XXIV | Itraconazole | Sodium sulfate 37%, | PS80 | 130 | 60-175 |
| | 50% | Leucine 8% | 5% | | |
| XXV | Itraconazole | Sodium sulfate 26%, | PS80 | 130 | 60-175 |
| | 60% | Leucine 8% | 6% | | |
| XXVI | Itraconazole | Sodium sulfate 15%, | PS80 | 130 | 60-175 |
| | 70% | Leucine 8% | 7% | | |
| XXVII | Itraconazole | Sodium sulfate | PS80 | 130 | 60-175 |
| | 75% | 9.5%, | 7.5% | | |
| | | Leucine 8% | | | |
| XXVIII | Itraconazole | Sodium sulfate 4%, | PS80 | 130 | 60-175 |
| | 80% | Leucine 8% | 8% | | |
| XXIX | Itraconazole | Sodium sulfate 10%, | PS80 | 130 | 60-175 |
| | 80% | Leucine 2% | 8% | | |
| XXX | Itraconazole | Sodium sulfate 11%, | PS80 | 130 | 60-175 |
| | 80% | Leucine 1% | 8% | | |

PS80 = polysorbate 80.

In a particular aspect, Formulation XII has an FPF less than 5 microns of the total dose of 57%, leading to a fine particle dose less than 5 microns of 2.8 mg for a 10.0 mg total dry powder capsule fill.

The dry powders and/or respirable dry particles described by any of the ranges or specifically disclosed formulations, characterized in the previous paragraph, may be filled into a receptacle, for example a capsule or a blister. When the receptacle is a capsule, the capsule is, for example, a size 2 or a size 3 capsule, and is preferably a size 3 capsule. The capsule material may be, for example, gelatin or HPMC (hydroxypropyl methylcellulose), and is preferably HPMC.

The dry powder and/or respirable dry particles described and characterized above may be contained in a dry powder inhaler (DPI). The DPI may be a capsule-based DPI or a blister-based DPI, and is preferably a capsule-based DPI. More preferably, the dry powder inhaler is selected from the RS01™ family of dry powder inhalers (Plastiape S.p.A., Italy). More preferably, the dry powder inhaler is selected from the RS01™ HR or the RS01™ UHR2. Most preferably, the dry powder inhaler is the RS01™ HR.

A dry powder for use in a method disclosed herein may comprise homogenous respirable dry particles that comprise itraconazole in crystalline particulate form, polysorbate 80, and or more excipients (e.g., a monovalent metal cation salt, e.g., a sodium salt), wherein the ratio of itraconazole to polysorbate 80 (wt:wt) in the dry powder is greater than 10:1, with the proviso that the dry powder does not comprise; 20% itraconazole, 39% sodium sulfate, 39% mannitol, and 2% polysorbate 80; 50% itraconazole, 22.5% sodium sulfate, 22.5% mannitol, and 5% polysorbate 80; 20% itraconazole, 62.4% sodium chloride, 15.6% leucine, and 2% polysorbate 80; 50% itraconazole, 36% sodium sulfate, 9% leucine, and 5% polysorbate 80; 20% itraconazole, 66.3% magnesium lactate, 11.7% leucine, and 2% polysorbate 80; 50% itraconazole, 38.25% magnesium lactate, 6.75% leucine, and 5% polysorbate 80; 50% itraconazole, 35% sodium sulfate, 10% leucine, and 5% polysorbate 80; 50% itraconazole, 35% sodium sulfate, 10% leucine, and less than 5% polysorbate 80; 50% itraconazole, 35% sodium sulfate, 13.75% leucine, and 1.25% polysorbate 80; 50% itraconazole, 37% sodium sulfate, 8% leucine, and 5% polysorbate 80; 60% itraconazole, 26% sodium sulfate, 8% leucine, and 6% polysorbate 80; 70% itraconazole, 15% sodium, 8% leucine, and 7% polysorbate 80; 75% itraconazole, 9.5% sodium sulfate, 8% leucine, and 7.5% polysorbate 80; 80% itraconazole, 4% sodium sulfate, 8% leucine, and 8% polysorbate 80; 80% itraconazole, 10% sodium sulfate, 2% leucine, and 8% polysorbate 80; or 80% itraconazole, 11% sodium sulfate, 1% leucine, and 8% polysorbate 80.

A dry powder useful in a method disclosed herein may comprise homogenous respirable dry particles that comprise itraconazole in crystalline particulate form, polysorbate 80, and one or more excipients (e.g., a monovalent metal cation salt, e.g., a sodium salt), wherein the ratio of itraconazole to polysorbate 80 (wt:wt) in the feedstock solution used for preparing the dry powder is greater than 10:1, with the proviso that the dry powder does not comprise; 20% itraconazole, 39% sodium sulfate, 39% mannitol, and 2% polysorbate 80; 50% itraconazole, 22.5% sodium sulfate, 22.5% mannitol, and 5% polysorbate 80; 20% itraconazole, 62.4% sodium chloride, 15.6% leucine, and 2% polysorbate 80; 50% itraconazole, 36% sodium sulfate, 9% leucine, and 5% polysorbate 80; 20% itraconazole, 66.3% magnesium lactate, 11.7% leucine, and 2% polysorbate 80; 50% itraconazole, 38.25% magnesium lactate, 6.75% leucine, and 5% polysorbate 80; 50% itraconazole, 35% sodium sulfate, 10% leucine, and 5% polysorbate 80; 50% itraconazole, 35% sodium sulfate, 10% leucine, and less than 5% polysorbate 80; 50% itraconazole, 35% sodium sulfate, 13.75% leucine, and 1.25% polysorbate 80; 50% itraconazole, 37% sodium sulfate, 8% leucine, and 5% polysorbate 80; 60% itraconazole, 26% sodium sulfate, 8% leucine, and 6% polysorbate 80; 70% itraconazole, 15% sodium, 8% leucine, and 7% polysorbate 80; 75% itraconazole, 9.5% sodium sulfate, 8% leucine, and 7.5% polysorbate 80; 80% itraconazole, 4% sodium sulfate, 8% leucine, and 8% polysorbate 80; 80% itraconazole, 10% sodium sulfate, 2% leucine, and 8% polysorbate 80; or 80% itraconazole, 11% sodium sulfate, 1% leucine, and 8% polysorbate 80.

Exemplary formulations that may be used in the methods described herein, include, but are not limited to, the following:

TABLE 1A
Additional exemplary dry powders comprising itraconazole.

| | | | Polysorbate | | Itraconazole |
|---|---|---|---|---|---|
| | | | 80 | Itraconazole: | subparticle |
| | Itraconazole | Excipients | (PS 80) | PS 80 | size range |
| Formulation | (wt %) | (wt %) | (wt %) | ratio | (Dv50 nm) |
| XXXI | Itraconazole | Sodium | PS 80 1.66% | 12:1 | 60-175 |
| | 20.0% | Sulfate | | | |
| | | 39.2%, | | | |
| | | Mannitol | | | |
| | | 39.2% | | | |
| XXXII | Itraconazole | Sodium | PS 80 4.17% | 12:1 | 60-175 |
| | 50.0% | Sulfate | | | |
| | | 22.9%, | | | |
| | | Mannitol | | | |
| | | 22.9% | | | |
| XXXIII | Itraconazole | Sodium | PS 80 4.17% | 12:1 | 60-175 |
| | 50.0% | Sulfate | | | |
| | | 45.8% | | | |
| XXXIV | Itraconazole | Sodium | PS 80 6.67% | 12:1 | 60-175 |
| | 80.0% | Sulfate | | | |
| | | 6.66%, | | | |
| | | Mannitol | | | |
| | | 6.67% | | | |
| XXXV | Itraconazole | Sodium | PS 80 6.67% | 12:1 | 60-175 |
| | 80.0% | Sulfate | | | |
| | | 13.3% | | | |
| XXXVI | Itraconazole | N/A | PS 80 7.69% | 12:1 | 60-175 |
| | 92.3% | | | | |
| XXXVII | Itraconazole | Sodium | PS 80 1.00% | 20:1 | 60-175 |
| | 20.0% | Sulfate | | | |
| | | 39.5%, | | | |
| | | Mannitol | | | |
| | | 39.5% | | | |
| XXXVIII | Itraconazole | Sodium | PS 80 2.50% | 20:1 | 60-175 |
| | 50.0% | Sulfate | | | |
| | | 23.8%, | | | |
| | | Mannitol | | | |
| | | 23.8% | | | |
| XXXIX | Itraconazole | Sodium | PS 80 4.00% | 20:1 | 60-175 |
| | 80.0% | Sulfate | | | |
| | | 8.00%, | | | |
| | | Mannitol | | | |
| | | 8.00% | | | |
| XXXX | Itraconazole | Sodium | PS 80 1.66% | 12:1 | 60-175 |
| | 20.0% | Sulfate | | | |
| | | 60.9%, | | | |
| | | Leucine | | | |
| | | 17.4% | | | |
| XXXXI | Itraconazole | Sodium | PS 80 4.16% | 12:1 | 60-175 |
| | 50.0% | Sulfate | | | |
| | | 35.7%, | | | |
| | | Leucine | | | |
| | | 10.2% | | | |
| XXXXII | Itraconazole | Sodium | PS 80 5.00% | 12:1 | 60-175 |
| | 60.0% | Sulfate | | | |
| | | 27.2%, | | | |
| | | Leucine | | | |
| | | 7.78% | | | |
| XXXXIII | Itraconazole | Sodium | PS 80 5.83% | 12:1 | 60-175 |
| | 70.0% | Sulfate | | | |
| | | 18.8%, | | | |
| | | Leucine | | | |
| | | 5.37% | | | |
| XXXXIV | Itraconazole | Sodium | PS 80 6.67% | 12:1 | 60-175 |
| | 80.0% | Sulfate | | | |
| | | 10.4%, | | | |
| | | Leucine | | | |
| | | 2.96% | | | |
| XXXXV | Itraconazole | Sodium | PS 80 6.67% | 12:1 | 60-175 |
| | 80.0% | Sulfate | | | |
| | | 6.67%, | | | |
| | | Leucine | | | |
| | | 6.66% | | | |
| XXXXVI | Itraconazole | Sodium | PS 80 6.67% | 12:1 | 60-175 |
| | 80.0% | Sulfate | | | |
| | | 2.96%, | | | |
| | | Leucine | | | |
| | | 10.4% | | | |
| XXXXVII | Itraconazole | Sodium | PS 80 1.00% | 20:1 | 60-175 |
| | 20.0% | Sulfate | | | |
| | | 61.4%, | | | |
| | | Leucine | | | |
| | | 17.6% | | | |
| XXXXVIII | Itraconazole | Sodium | PS 80 2.50% | 20:1 | 60-175 |
| | 50.0% | Sulfate | | | |
| | | 36.9%, | | | |
| | | Leucine | | | |
| | | 10.6% | | | |
| XLIX | Itraconazole | Sodium | PS 80 2.50% | 20:1 | 60-175 |
| | 50.0% | Sulfate | | | |
| | | 47.5% | | | |
| L | Itraconazole | Sodium | PS 80 3.00% | 20:1 | 60-175 |
| | 60.0% | Sulfate | | | |
| | | 28.8%, | | | |
| | | Leucine | | | |
| | | 8.20% | | | |
| LI | Itraconazole | Sodium | PS 80 3.50% | 20:1 | 60-175 |
| | 70.0% | Sulfate | | | |
| | | 20.6%, | | | |
| | | Leucine | | | |
| | | 5.89% | | | |
| LII | Itraconazole | Sodium | PS 80 4.00% | 20:1 | 60-175 |
| | 80.0% | Sulfate | | | |
| | | 12.4%, | | | |
| | | Leucine | | | |
| | | 3.56% | | | |
| LIII | Itraconazole | N/A | PS 80 4.76% | 20:1 | 60-175 |
| | 95.2% | | | | |
| LIV | Itraconazole | Sodium | PS 80 | 30:1 | 60-175 |
| | 20.0% | Sulfate | 0.667% | | |
| | | 61.7%, | | | |
| | | Leucine | | | |
| | | 17.6% | | | |
| LV | Itraconazole | Sodium | PS 80 1.67% | 30:1 | 60-175 |
| | 50.0% | Sulfate | | | |
| | | 37.6%, | | | |
| | | Leucine | | | |
| | | 10.7% | | | |
| LVI | Itraconazole | Sodium | PS 80 2.00% | 30:1 | 60-175 |
| | 60% | Sulfate | | | |
| | | 29.6%, | | | |
| | | Leucine | | | |
| | | 8.44% | | | |
| LVII | Itraconazole | Sodium | PS 80 2.33% | 30:1 | 60-175 |
| | 70% | Sulfate | | | |
| | | 21.5%, | | | |
| | | Leucine | | | |
| | | 6.15% | | | |
| LVIII | Itraconazole | Sodium | PS 80 2.67% | 30:1 | 60-175 |
| | 80% | Sulfate | | | |
| | | 13.5%, | | | |
| | | Leucine | | | |
| | | 3.85% | | | |

PS 80 = polysorbate 80.

### Methods for Preparing Dry Powders and Dry Particles

The respirable dry particles and dry powders used in a method disclosed herein can be prepared using any suitable method, with the proviso that the dry powders cannot be an extemporaneous dispersion. Many suitable methods for preparing dry powders and/or respirable dry particles are conventional in the art, and include single and double emulsion solvent evaporation, spray drying, spray-freeze drying, milling (e.g., jet milling), blending, solvent extraction, solvent evaporation, phase separation, simple and complex coacervation, interfacial polymerization, suitable methods that involve the use of supercritical carbon dioxide (CO₂), sonocrystalliztion, nanoparticle aggregate formation and other suitable methods, including combinations thereof. Respirable dry particles can be made using methods for making microspheres or microcapsules known in the art. These methods can be employed under conditions that result in the formation of respirable dry particles with desired aerodynamic properties (e.g., aerodynamic diameter and geometric diameter). If desired, respirable dry particles with desired properties, such as size and density, can be selected using suitable methods, such as sieving.

Suitable methods for selecting respirable dry particles with desired properties, such as size and density, include wet sieving, dry sieving, and aerodynamic classifiers (such as cyclones).

The respirable dry particles are preferably spray dried. Suitable spray-drying techniques are described, for example, by K. Masters in “Spray Drying Handbook”, John Wiley & Sons, New York (1984). Generally, during spray-drying, heat from a hot gas such as heated air or nitrogen is used to evaporate a solvent from droplets formed by atomizing a continuous liquid feed. When hot air is used, the moisture in the air is at least partially removed before its use. When nitrogen is used, the nitrogen gas can be run “dry”, meaning that no additional water vapor is combined with the gas. If desired the moisture level of the nitrogen or air can be set before the beginning of spray dry run at a fixed value above “dry” nitrogen. If desired, the spray drying or other instruments, e.g., jet milling instrument, used to prepare the dry particles can include an inline geometric particle sizer that determines a geometric diameter of the respirable dry particles as they are being produced, and/or an inline aerodynamic particle sizer that determines the aerodynamic diameter of the respirable dry particles as they are being produced.

For spray drying, solutions, emulsions or suspensions that contain the components of the dry particles to be produced in a suitable solvent (e.g., aqueous solvent, organic solvent, aqueous-organic mixture or emulsion) are distributed to a drying vessel via an atomization device. For example, a nozzle or a rotary atomizer may be used to distribute the solution or suspension to the drying vessel. The nozzle can be a two-fluid nozzle, which can be in an internal mixing setup or an external mixing setup. Alternatively, a rotary atomizer having a 4- or 24-vaned wheel may be used. Examples of suitable spray dryers that can be outfitted with a rotary atomizer and/or a nozzle, include, a Mobile Minor Spray Dryer or the Model PSD-1, both manufactured by GEA Niro, Inc. (Denmark), Büchi B-290 Mini Spray Dryer (BÜCHI Labortechnik AG, Flawil, Switzerland), ProCepT Formatrix R&D spray dryer (ProCepT nv, Zelzate, Belgium), among several other spray dryer options. Actual spray drying conditions will vary depending, in part, on the composition of the spray drying solution or suspension and material flow rates. The person of ordinary skill will be able to determine appropriate conditions based on the compositions of the solution, emulsion or suspension to be spray dried, the desired particle properties and other factors. In general, the inlet temperature to the spray dryer is about 90° C. to about 300° C. The spray dryer outlet temperature will vary depending upon such factors as the feed temperature and the properties of the materials being dried. Generally, the outlet temperature is about 50° C. to about 150° C. If desired, the respirable dry particles that are produced can be fractionated by volumetric size, for example, using a sieve, or fractioned by aerodynamic size, for example, using a cyclone, and/or further separated according to density using techniques known to those of skill in the art.

To prepare the respirable dry particles, generally, an emulsion or suspension that contains the desired components of the dry powder (i.e., a feedstock) is prepared and spray dried under suitable conditions. Preferably, the dissolved or suspended solids concentration in the feedstock is at least about 1 g/L, at least about 2 g/L, at least about 5 g/L, at least about 10 g/L, at least about 15 g/L, at least about 20 g/L, at least about 30 g/L, at least about 40 g/L, at least about 50 g/L, at least about 60 g/L, at least about 70 g/L, at least about 80 g/L, at least about 90 g/L or at least about 100 g/L. The feedstock can be provided by preparing a single solution, suspension or emulsion by dissolving, suspending, or emulsifying suitable components (e.g., salts, excipients, other active ingredients) in a suitable solvent. The solution, emulsion or suspension can be prepared using any suitable methods, such as bulk mixing of dry and/or liquid components or static mixing of liquid components to form a combination. For example, a hydrophilic component (e.g., an aqueous solution) and a hydrophobic component (e.g., an organic solution) can be combined using a static mixer to form a combination. The combination can then be atomized to produce droplets, which are dried to form respirable dry particles. Preferably, the atomizing step is performed immediately after the components are combined in the static mixer. Alternatively, the atomizing step is performed on a bulk mixed solution.

The feedstock can be prepared using any solvent in which the angiogenesis inhibitor in particulate form has low solubility, such as an organic solvent, an aqueous solvent or mixtures thereof. Suitable organic solvents that can be employed include but are not limited to alcohols such as, for example, ethanol, methanol, propanol, isopropanol, butanols, and others. Other organic solvents include but are not limited to tetrahydrofuran (THF), perfluorocarbons, dichloromethane, chloroform, ether, ethyl acetate, methyl tert-butyl ether and others. Co-solvents that can be employed include an aqueous solvent and an organic solvent, such as, but not limited to, the organic solvents as described above. Aqueous solvents include water and buffered solutions. A preferred solvent is water.

Various methods (e.g., static mixing, bulk mixing) can be used for mixing the solutes and solvents to prepare feedstocks, which are known in the art. If desired, other suitable methods of mixing may be used. For example, additional components that cause or facilitate the mixing can be included in the feedstock. For example, carbon dioxide produces fizzing or effervescence and thus can serve to promote physical mixing of the solute and solvents.

The feedstock or components of the feedstock can have any desired pH, viscosity or other properties. If desired, a pH buffer can be added to the solvent or co-solvent or to the formed mixture. Generally, the pH of the mixture ranges from about 3 to about 8.

Dry powder and/or respirable dry particles can be fabricated and then separated, for example, by filtration or centrifugation by means of a cyclone, to provide a particle sample with a preselected size distribution. For example, greater than about 30%, greater than about 40%, greater than about 50%, greater than about 60%, greater than about 70%, greater than about 80%, or greater than about 90% of the respirable dry particles in a sample can have a diameter within a selected range. The selected range within which a certain percentage of the respirable dry particles fall can be, for example, any of the size ranges described herein, such as between about 0.1 to about 3 microns VMGD.

The suspension may be a nano-suspension, similar to an intermediate for making dry powder comprising nano-crystalline compound.

The dry powder may be a drug embedded in a matrix material, such as sodium sulfate and leucine. Optionally, the dry powder may be spray dried such that the dry particles are small, dense, and dispersible.

The dry powders can consist solely of the respirable dry particles described herein without other carrier or excipient particles (referred to as “neat powders”). If desired the dry powders can comprise blends of the respirable dry particles described herein and other carrier or excipient particles, such as lactose carrier particles that are greater than 10 microns, 20 microns to 500 microns, and preferably between 25 microns and 250 microns. In some embodiments, dry powders comprising carrier particles (blended powders) are excluded.

In a preferred embodiment, the dry powders do not comprise carrier particles. In one aspect, the angiogenesis inhibitor is embedded in a matrix comprising excipient and/or stabilizer. The dry powder may comprise respirable dry particles of uniform content, wherein each particle comprises the angiogenesis inhibitor. Thus, as used herein, “uniform content” means that every respirable particle comprises some amount of angiogenesis inhibitor, with the optional stabilizer and/or excipient.

The dry powders can comprise respirable dry particles wherein at least 98%, at least 99%, or substantially all of the particles (by weight) comprise an angiogenesis inhibitor.

The dry powders can comprise angiogenesis inhibitor distributed throughout a matrix comprising one or more excipients. The excipients can comprise any number of salts, sugars, lipids, amino acids, surfactants, polymers, or other components suitable for pharmaceutical use. Preferred excipients include sodium sulfate and leucine. The dry powders are typically manufactured by first processing the angiogenesis inhibitor (e.g., itraconazole, such as itraconazole in crystalline form) to adjust the particle size using any number of techniques that are familiar to those of skill in the art (e.g., wet milling, jet milling). For example, a crystalline angiogenesis inhibitor may be processed in an antisolvent with a stabilizer to form a suspension. Preferred stabilizers include polysorbates (also known as TWEEN®), such as polysorbate 80 (PS80). Another preferred stabilizer is oleic acid, or a salt thereof. The stabilized suspension of crystalline angiogenesis inhibitor is then spray dried with the one or more additional excipients. The resulting dry particles comprise crystalline angiogenesis inhibitor dispersed throughout an excipient matrix with each dry particle having a homogenous composition.

In a particular embodiment, a dry powder of the present invention is made by starting with crystalline angiogenesis inhibitor (e.g., itraconazole), which is usually obtainable in a micro-crystalline size range. The particle size of the micro-crystalline angiogenesis inhibitor is reduced into the nano-crystalline size using any of a number of techniques familiar to those of skill in the art, including but not limited to, high-pressure homogenization, high-shear homogenization, jet-milling, pin milling, microfluidization, or wet milling (also known as ball milling, pearl milling or bead milling). Wet milling is often preferred, as it is able to achieve a wide range of particle size distributions, including those in the nanometer (<1 μm) size domain. What becomes especially important in the sub-micron size domain is the use of surface stabilizing components, such as surfactants (e.g., polysorbate 80, also known as TWEEN® 80). Surfactants enable the creation of submicron particles during milling and the formation of physically stable suspensions, as they sequester the many high energy surfaces created during milling preventing aggregation and sedimentation. Thus, the presence of the surfactant is important to spray drying homogenous micro-particles as the surfactant allows for the formation of a uniform and stable suspension ensuring compositional homogeneity across particles. The use of surfactant allows for formation of micro-suspension or nano-suspensions. With the surfactant, the nano-crystalline angiogenesis inhibitor (e.g., itraconazole) particles are suspended in a stable colloidal suspension in the anti-solvent. The anti-solvent for the drug can utilize water, or a combination of water and other miscible solvents such as alcohols or ketones as the continuous anti-solvent phase for the colloidal suspension. A spray drying feedstock may be prepared by dissolving the soluble components in a desired solvent(s) followed by dispersing the surfactant-stabilized crystalline angiogenesis inhibitor nanosuspension in the resulting feedstock while mixing, although the process is not limited to this specific order of operations.

Methods for analyzing the dry powders and/or respirable dry particles are found in the Exemplification section below.

### Liquid Formulations

Liquid formulations for delivery with a pressurized metered dose inhaler (pMDI) or with a soft mist inhaler (SMI) can be prepared using any suitable method. For example, for use with a pMDI, a feedstock may be prepared inside a pressurized canister in which an angiogenesis inhibitor (e.g., itraconazole) in crystalline particulate form is suspended in a propellant such as a HFA propellant or a CFC propellant, optionally stabilized with a stabilizer such as polysorbate 80. The pressurized suspension may then be delivered into the respiratory tract of a subject by actuating the pMDI. Table 1B contains various embodiments for delivery of itraconazole in crystalline particulate form by use of the pMDI. The nanoparticle solids concentration may vary from about 5%, about 10%, about 15%, about 20%, about 25%, about 30%, about 35%, about 40%, or about 50%. The dose volume of the pMDI may vary from about 20 μL to about 110 μL. The amount of itraconazole in the dose volume may be about 15%, 20%, 25%, 30% or 40%. The remainder of the volume may comprise propellant and optionally a surfactant. The pMDI delivery efficiency may be about 15%, 20%, 25%, 30% or 40%. Nominal doses of itraconazole in a pMDI may be varied from about 0.50 mg to about 12 mg. For example, the nominal dose may be about 2 mg, about 3 mg, about 4 mg, about 5 mg, about 6 mg, about 7 mg, about 8 mg, about 9 mg, about 10 mg or about 12 mg. The calculated delivery dose may range from about 0.1 mg to about 5 mg.

TABLE 1B
Pressurized Metered Dose Inhaler (pMDI)

| | Dose | Drug | | Nominal | Delivered |
|---|---|---|---|---|---|
| Nanoparticle | volume | amount | pMDI | dose | dose |
| solids | from | in dose | delivery | from | from |
| concentration | pMDI | volume | efficiency | pMDI | PMDI |
| (%) | (uL) | (%) | (%) | (mg) | (mg) |
| 10 | 25 | 20 | 30 | 0.50 | 0.15 |
| 10 | 25 | 30 | 20 | 0.75 | 0.15 |
| 10 | 25 | 30 | 30 | 0.75 | 0.23 |
| 10 | 100 | 20 | 30 | 2.00 | 0.60 |
| 10 | 100 | 30 | 20 | 3.00 | 0.60 |
| 10 | 100 | 30 | 30 | 3.00 | 0.90 |
| 25 | 25 | 20 | 30 | 1.25 | 0.38 |
| 25 | 25 | 30 | 20 | 1.88 | 0.38 |
| 25 | 25 | 30 | 30 | 1.88 | 0.56 |
| 25 | 100 | 20 | 30 | 5.00 | 1.50 |
| 25 | 100 | 30 | 20 | 7.50 | 1.50 |
| 25 | 100 | 30 | 30 | 7.50 | 2.25 |
| 35 | 25 | 20 | 30 | 1.75 | 0.53 |
| 35 | 25 | 30 | 20 | 2.63 | 0.53 |
| 35 | 25 | 30 | 30 | 2.63 | 0.79 |
| 35 | 100 | 20 | 30 | 7.00 | 2.10 |
| 35 | 100 | 30 | 20 | 10.50 | 2.10 |
| 35 | 100 | 30 | 30 | 10.50 | 3.15 |

| | | |
|---|---|---|
| density of water: | 1 | g/mL |
| Unit conversion: | 1000 | mg/g |
| Unit conversion: | 1000 | uL/mL |

For use with a soft mist inhaler (SMI), for example, a feedstock may be prepared in which an angiogenesis inhibitor (e.g., itraconazole) in crystalline particulate form is suspended in a solvent such as water in which the angiogenesis inhibitor is poorly soluble, and stabilized with a stabilizer, such as polysorbate 80. The suspension may be stored in a collapsible bag inside a cartridge which is loaded inside the device. A forced metered volume of suspension proceeds through a capillary tube into a micropump. Upon actuation of the SMI, a dose may be delivered to a patient. Table IC contains various embodiments for delivery of itraconazole in crystalline particulate form by use of the SMI. The nanoparticle solids concentration vary from about 5%, about 10%, about 15%, about 20%, about 25%, about 30%, about 35%, about 40%, or about 50%. The dose volume of the SMI may vary from about 10 uL to about 25 uL. The formulation may comprise itraconazole in crystalline particulate form and surfactant. The SMI delivery efficiency may be about 65%, 70%, 75%, 80%, or 85%. Nominal doses of itraconazole in a pMDI may vary from about 1.0 mg to about 8 mg. For example, the nominal dose may be about 2 mg, about 3 mg, about 4 mg, about 5 mg, about 6 mg, about 7 mg, or about 8 mg. The calculated delivery dose may range from about 0.5 mg to about 5 mg.

TABLE 1C
Soft Mist Inhaler

| | Dose | | Nominal | Delivered |
|---|---|---|---|---|
| Nanoparticle | volume | pMDI | dose | dose |
| solids | from | delivery | from | from |
| concentration | SMI | efficiency | pMDI | pMDI |
| (%) | (uL) | (%) | (mg) | (mg) |
| 10 | 15 | 75 | 1.50 | 1.13 |
| 25 | 15 | 75 | 3.75 | 2.81 |
| 35 | 15 | 75 | 5.25 | 3.94 |

| | | |
|---|---|---|
| density of water: | 1 | g/mL |
| Unit conversion: | 1000 | mg/g |
| Unit conversion: | 1000 | uL/mL |

### EXAMPLES

Materials used in the following Examples and their sources are listed below. Sodium chloride, sodium sulfate, polysorbate 80, oleic acid, ammonium hydroxide, mannitol, magnesium lactate, and L-leucine were obtained from Sigma-Aldrich Co. (St. Louis, MO), Spectrum Chemicals (Gardena, CA), Applichem (Maryland Heights, MO), Alfa Aesar (Tewksbury, MA), Thermo Fisher (Waltham, MA), Croda Chemicals (East Yorkshire, United Kingdom) or Merck (Darmstadt, Germany). Itraconazole was obtained from Neuland (Princeton, NJ) or SMS Pharmaceutical ltd (Telengana State, India). Ultrapure (Type II ASTM) water was from a water purification system (Millipore Corp., Billerica, MA), or equivalent.

### Methods:

Geometric of Volume Diameter of Suspensions. Volume median diameter (×50 or Dv50), which may also be referred to as volume median geometric diameter (VMGD), of the active agent suspensions was determined using a laser diffraction technique. The equipment consisted of a Horiba LA-950 instrument outfitted with an automated recirculation system for sample handling and removal or a fixed-volume sample cuvette. The sample to a dispersion media, consisting of either deionized water or deionized water with less than 0.5% of a surfactant such as polysorbate 80 or sodium dodecyl sulfate. Ultrasonic energy can be applied to aid in dispersion of the suspension. When the laser transmission was in the correct range, the sample was sonicated for 60 seconds at a setting of 5. The sample was then measured and the particle size distribution reported.

Geometric or Volume Diameter of Dry Powders. Volume median diameter (×50 or Dv50), which may also be referred to as volume median geometric diameter (VMGD), of the dry powders was determined using a laser diffraction technique. The equipment consisted of a HELOS diffractometer and a RODOS dry powder disperser (Sympatec, Inc., Princeton, NJ). The RODOS disperser applies a shear force to a sample of particles, controlled by the regulator pressure (typically set at 1.0 bar with maximum orifice ring pressure) of the incoming compressed dry air. The pressure settings may be varied to vary the amount of energy used to disperse the powder. For example, the dispersion energy may be modulated by changing the regulator pressure from 0.2 bar to 4.0 bar. Powder sample is dispensed from a microspatula into the RODOS funnel. The dispersed particles travel through a laser beam where the resulting diffracted light pattern produced is collected, typically using an R1 lens, by a series of detectors. The ensemble diffraction pattern is then translated into a volume-based particle size distribution using the Fraunhofer diffraction model, on the basis that smaller particles diffract light at larger angles. Using this method, the span of the distribution was also determined per the formula (Dv[90]−Dv[10)/Dv[50]. The span value gives a relative indication of the polydispersity of the particle size distribution.

Aerodynamic Performance via Andersen Cascade Impactor The aerodynamic properties of the powders dispersed from an inhaler device were assessed with an Mk-II 1 ACFM Andersen Cascade Impactor (Copley Scientific Limited. Nottingham, UK) (ACI). The ACI instrument was run in controlled environmental conditions of 18 to 25° C. and relative humidity (RH) between 25 and 35%. The instrument consists of eight stages that separate aerosol particles based on inertial impaction. At each stage, the aerosol stream passes through a set of nozzles and impinges on a corresponding impaction plate. Particles having small enough inertia will continue with the aerosol stream to the next stage, while the remaining particles will impact upon the plate. At each successive stage, the aerosol passes through nozzles at a higher velocity and aerodynamically smaller particles are collected on the plate. After the aerosol passes through the final stage, a filter collects the smallest particles that remain, called the “final collection filter”. Gravimetric and/or chemical analyses can then be performed to determine the particle size distribution. A short stack cascade impactor, also referred to as a collapsed cascade impactor, is also utilized to allow for reduced labor time to evaluate two aerodynamic particle size cut-points. With this collapsed cascade impactor, stages are eliminated except those required to establish fine and coarse particle fractions. The impaction techniques utilized allowed for the collection of two or eight separate powder fractions. The capsules (HPMC, Size 3; Capsugel Vcaps, Peapack, NJ) were filled with powder to a specific weight and placed in a hand-held, breath-activated dry powder inhaler (DPI) device, the high resistance RS01™ DPI or the ultra-high resistance UHR2 DPI (both by Plastiape, Osnago, Italy). The capsule was punctured and the powder was drawn through the cascade impactor operated at a flow rate of 60.0 L/min for 2.0 s. At this flowrate, the calibrated cut-off diameters for the eight stages are 8.6, 6.5, 4.4, 3.3, 2.0, 1.1, 0.5 and 0.3 microns and for the two stages used with the short stack cascade impactor, based on the Andersen Cascade Impactor, the cut-off diameters are 5.6 microns and 3.4 microns. The fractions were collected by placing filters in the apparatus and determining the amount of powder that impinged on them by gravimetric measurements or chemical measurements on an HPLC.

Aerodynamic Performance via Next Generation Impactor. The aerodynamic properties of the powders dispersed from an inhaler device were assessed with a Next Generation Impactor (Copley Scientific Limited, Nottingham, UK) (NGI). For measurements utilizing the NGI, the NGI instrument was run in controlled environmental conditions of 18 to 25° C. and relative humidity (RH) between 25 and 35%. The instrument consists of seven stages that separate aerosol particles based on inertial impaction and can be operated at a variety of air flow rates. At each stage, the aerosol stream passes through a set of nozzles and impinges on a corresponding impaction surface. Particles having small enough inertia will continue with the aerosol stream to the next stage, while the remaining particles will impact upon the surface. At each successive stage, the aerosol passes through nozzles at a higher velocity and aerodynamically smaller particles are collected on the plate. After the aerosol passes through the final stage, a micro-orifice collector collects the smallest particles that remain. Gravimetric and/or chemical analyses can then be performed to determine the particle size distribution. The capsules (HPMC, Size 3; Capsugel Vcaps, Peapack, NJ) were filled with powder to a specific weight and placed in a hand-held, breath-activated dry powder inhaler (DPI) device, the high resistance RS01 DPI or the ultra-high resistance RS01 DPI (both by Plastiape, Osnago, Italy). The capsule was punctured and the powder was drawn through the cascade impactor operated at a specified flow rate for 2.0 Liters of inhaled air. At the specified flow rate, the cut-off diameters for the stages were calculated. The fractions were collected by placing wetted filters in the apparatus and determining the amount of powder that impinged on them by chemical measurements on an HPLC.

Fine Particle Dose The fine particle dose indicates the mass of one or more therapeutics in a specific size range and can be used to predict the mass which will reach a certain region in the respiratory tract. The fine particle dose can be measured gravimetrically or chemically via either an ACI or NGI. If measured gravimetrically, since the dry particles are assumed to be homogenous, the mass of the powder on each stage and collection filter can be multiplied by the fraction of therapeutic agent in the formulation to determine the mass of therapeutic. If measured chemically, the powder from each stage or filter is collected, separated, and assayed for example on an HPLC to determine the content of the therapeutic. The cumulative mass deposited on each of the stages at the specified flow rate is calculated and the cumulative mass corresponding to a 5.0 micrometer diameter particle is interpolated. This cumulative mass for a single dose of powder, contained in one or more capsules, actuated into the impactor is equal to the fine particle dose less than 5.0 microns (FPD <5.0 microns).

Mass Median Aerodynamic Diameter. Mass median aerodynamic diameter (MMAD) was determined using the information obtained by the Andersen Cascade Impactor (ACI). The cumulative mass under the stage cut-off diameter is calculated for each stage and normalized by the recovered dose of powder. The MMAD of the powder is then calculated by linear interpolation of the stage cut-off diameters that bracket the 50th percentile. An alternative method of measuring the MMAD is with the Next Generation Impactor (NGI). Like the ACI, the MMAD is calculated with the cumulative mass under the stage cut-off diameter is calculated for each stage and normalized by the recovered dose of powder. The MMAD of the powder is then calculated by linear interpolation of the stage cut-off diameters that bracket the 50th percentile.

Emitted Geometric or Volume Diameter. The volume median diameter (Dv50) of the powder after it is emitted from a dry powder inhaler, which may also be referred to as volume median geometric diameter (VMGD), was determined using a laser diffraction technique via the Spraytec diffractometer (Malvern, Inc.). Powder was filled into size 3 capsules (V-Caps, Capsugel) and placed in a capsule based dry powder inhaler (RS01™ Model 7 High resistance. Plastiape, Italy), or DPI, and the DPI sealed inside a cylinder. The cylinder was connected to a positive pressure air source with steady air flow through the system measured with a mass flow meter and its duration controlled with a timer controlled solenoid valve. The exit of the dry powder inhaler was exposed to room pressure and the resulting aerosol jet passed through the laser of the diffraction particle sizer (Spraytec) in its open bench configuration before being captured by a vacuum extractor. The steady air flow rate through the system was initiated using the solenoid valve. A steady air flow rate was drawn through the DPI typically at 60 L/min for a set duration, typically of 2 seconds. Alternatively, the air flow rate drawn through the DPI was sometimes run at 15 L/min, 20 L/min, or 30 L/min. The resulting geometric particle size distribution of the aerosol was calculated from the software based on the measured scatter pattern on the photodetectors with samples typically taken at 1000 Hz for the duration of the inhalation. The Dv50, GSD, FPF<5.0 μm measured were then averaged over the duration of the inhalation.

The Emitted Dose (ED) refers to the mass of therapeutic which exits a suitable inhaler device after a firing or dispersion event. The ED is determined using a method based on USP Section 601 Aerosols, Metered-Dose Inhalers and Dry Powder Inhalers, Delivered-Dose Uniformity, Sampling the Delivered Dose from Dry Powder Inhalers. United States Pharmacopeia convention, Rockville, MD, 13th Revision, 222-225, 2007. Contents of capsules are dispersed using either the RS01 HR inhaler at a pressure drop of 4 kPa and a typical flow rate of 60 LPM or the UHR2 RS01 at a pressure drop of 4 kPa and a typical flow rate of 39 LPM. The emitted powder is collected on a filter in a filter holder sampling apparatus. The sampling apparatus is rinsed with a suitable solvent such as water and analyzed using an HPLC method. For gravimetric analysis a shorter length filter holder sampling apparatus is used to reduce deposition in the apparatus and the filter is weighed before and after to determine the mass of powder delivered from the DPI to the filter. The emitted dose of therapeutic is then calculated based on the content of therapeutic in the delivered powder. Emitted dose can be reported as the mass of therapeutic delivered from the DPI or as a percentage of the filled dose.

Thermogravimetric Analysis; Thermogravimetric analysis (TGA) was performed using either the Q500 model or the Discovery model thermogravimetric analyzer (TA Instruments, New Castle, DE). The samples were either placed into an open aluminum DSC pan or a sealed aluminum DSC pan that was then automatically punched open prior to the time of test. Tare weights were previously recorded by the instrument. The following method was employed; Ramp 5.00° C./min from ambient (˜35° C.) to 200° C. The weight loss was reported as a function of temperature up to 140° C. TGA allows for the calculation of the content of volatile compounds within the dry powder. When utilizing processes with water alone, or water in conjunction with volatile solvents, the weight loss via TGA is a good estimate of water content.

X-Ray Powder Diffraction; The crystalline character of the formulations was assessed via powder X-ray diffraction (PXRD). A 20-30 mg sample of material is analyzed in a powder X-ray diffractometer (D8 Discover with LINXEYE detector; Bruker Corporation, Billerica, MA or equivalent) using a Cu X-ray tube with 1.5418A at a data accumulation time 1.2 second/step over a scan range of 5 to 45° 20 and a step size of 0.02° 20.

Itraconazole Content/Purity using HPLC. A high performance liquid chromatography (HPLC) method utilizing a reverse phase C18 column coupled to an ultraviolet (UV) detector has been developed for the identification, bulk content, assay, CUPMD and impurities analysis of itraconazole dry powders. The reverse phase column is equilibrated to 30° C. and the autosampler is set to 5° C. The mobile phases, 20 mM sodium phosphate monobasic at a pH of 2.0 (mobile phase A) and acetonitrile (mobile phase B) are used in a gradient elution from a ratio of 59:41 (A:B) to 5:95 (A:B), over the course of a 19.5 minute run time. Detection is by UV at 258 nm and the injection volume is 10 μL. Itraconazole content in powders are quantified relative to a standard curve.

Identification of known impurities A, B, C, D, E, F and G (shown in monograph Ph. Eur. 01/2011:1335) is confirmed by comparing the retention time of the impurity peaks in the itraconazole dry powder samples to that of the itraconazole USP impurity mix reference standard spiked with impurity A. Unknown impurities are identified and quantified by relative retention time to that of the itraconazole main peak and with area above the limit of detection (LOD). All impurities are measured by area percent, with respect to the itraconazole peak.

Particle Size Reduction. The particle size distribution of the crystalline active agent can be modulated using a number of techniques familiar to those of skill in the art, including but not limited to, high-pressure homogenization, high-shear homogenization, jet-milling, pin milling, microfluidization, or wet milling (also known as ball milling, pearl milling or bead milling). Wet milling is often preferred, as it is able to achieve a wide range of particle size distributions, including those in the nanometer (<1 μm) size domain.

Particle Size Reduction using Low Energy Wet Milling. One technique for reducing the particle size of the active agent was via low energy wet milling (also known as roller milling, or jar milling). Suspensions of the active agent were prepared in an anti-solvent, which can be water, or any solvent in which the active agent is not appreciably soluble. Stabilizers, which can be, but are not limited to, non-ionic surfactants or amphiphilic polymers, are then added to the suspension along with milling media, which can be, but are not limited to, spherical with high wear resistance and in the size range from 0.03 to 0.70 millimeters in diameter. The vessels containing the suspensions are then rotated using a jar mill (US Stoneware, East Palestine, OH USA) while taking samples periodically to assess particle size (LA-950, HORIBA, Kyoto, Japan). When the particle size is sufficiently reduced, or when a particle size minimum is reached, the suspension is strained through a sieve to remove the milling media, and the product recovered.

Particle Size Reduction using High Energy Wet Milling. Another technique for reducing the particle size of the active agent was via high-energy wet milling using a rotor-stator, or agitated media mill. Suspensions of the active agent were prepared in an anti-solvent, which can be water, or any solvent in which the active agent is not appreciably soluble. Stabilizers, which can be, but are not limited to, non-ionic surfactants or amphiphilic polymers, are then added to the suspension along with milling media, which can be, but are not limited to, spherical with high wear resistance and in the size range from 0.03 to 0.70 millimeters in diameter. The suspensions are then charged into the mill, which can be operated in either batch or recirculation mode. The process consists of the suspension and milling media being agitated within the milling chamber, which increases the energy input to the system and accelerates the particle size reduction process. The milling chamber and recirculation vessel are jacketed and actively cooled to avoid temperature increases in the product. The agitation rate and recirculation rate of the suspension are controlled during the process. Samples are taken periodically to assess particle size (LA-950, HORIBA, Kyoto, Japan). When the particle size is sufficiently reduced, or when a particle size minimum is reached, the suspension is discharged from the mill.

Particle Size Reduction using Microfluidization. Another technique for reducing the particle size distribution of the active agent was via Microfluidization. Microfluidizer-based processing is a high-shear wet-processing unit operation utilized for particle size reduction of liquids and solids. The unit can be configured with various interaction chambers, which are cylindrical modules with specific orifice and channel designs through which fluid is passed at high pressures to control shear rates. Product enters the unit via the inlet reservoir and is forced into the fixed-geometry interaction chamber at speeds up to 400 m/sec by a high-pressure pump. It is then effectively cooled, if required, and collected in the output reservoir. The process can be repeated as necessary (e.g. multiple “passes”) to achieve the particle size targets. Particle size of the active agent is monitored periodically via laser diffraction (LA-950, HORIBA, Kyoto, Japan). When the particle size is sufficiently reduced, or when a particle size minimum is reached, the suspension is recovered from the unit.

Particle Size Reduction using Jet Milling Another technique for reducing the particle size distribution of the active agent was via jet milling. Jet mills utilize fluid energy (compressed air or gas) to grind and classify, in a single chamber with no moving parts. Activated by high pressure air, the particles are accelerated into a high speed rotation in a shallow grinding chamber. As the particles impact on one another their size is reduced. Centrifugal force holds larger particles in the grinding rotation area until they have achieved the desired fine particle size. Centripetal force drags the desired particles towards the static classifier where they are allowed to exit upon achieving the correct particle size. The final particle size is controlled by varying the rate of the feed and propellant pressure.

Liquid Feedstock Preparation for Spray Drying. Spray drying homogenous particles requires that the ingredients of interest be solubilized in solution or suspended in a uniform and stable suspension. The feedstock can utilize water, or a combination of water and other miscible solvents such as alcohols or ketones, as the solvent in the case of solutions, or as the continuous phase in the case of suspensions. Feedstocks of the various formulations were prepared by dissolving the soluble components in the desired solvent(s) followed by dispersing the surfactant-stabilized active agent-containing suspension in the resulting solution while mixing, although the process is not limited to this specific order of operations.

Spray Drying Using Niro Spray Dryer. Dry powders were produced by spray drying utilizing a Niro Mobile Minor spray dryer (GEA Process Engineering Inc., Columbia, MD) with powder collection from a cyclone, a product filter or both. Atomization of the liquid feed was performed using a co-current two-fluid nozzle either from Niro (GEA Process Engineering Inc., Columbia, MD) or a Spraying Systems (Carol Stream, IL) 1/4J two-fluid nozzle with gas cap 67147 and fluid cap 2850SS, although other two-fluid nozzle setups are also possible. In some embodiments, the two-fluid nozzle can be in an internal mixing setup or an external mixing setup. Additional atomization techniques include rotary atomization or a pressure nozzle. The liquid feed was fed using gear pumps (Cole-Parmer Instrument Company, Vernon Hills, IL) directly into the two-fluid nozzle or into a static mixer (Charles Ross & Son Company, Hauppauge, NY) immediately before introduction into the two-fluid nozzle. An additional liquid feed technique includes feeding from a pressurized vessel. Nitrogen or air may be used as the drying gas, provided that moisture in the air is at least partially removed before its use. Pressurized nitrogen or air can be used as the atomization gas feed to the two-fluid nozzle. The drying gas inlet temperature can range from 70° C. to 300° C. and outlet temperature from 30° C. to 120° C. with a liquid feedstock rate of 10 mL/min to 100 mL/min. The gas supplying the two-fluid atomizer can vary depending on nozzle selection and for the Niro co-current two-fluid nozzle can range from 5 kg/hr to 50 kg/hr or for the Spraying Systems 1/4J two-fluid nozzle can range from 30 g/min to 150 g/min. The atomization gas rate can be set to achieve a certain gas to liquid mass ratio, which directly affects the droplet size created. The pressure inside the drying drum can range from +3 “WC to −6 “WC. Spray dried powders can be collected in a container at the outlet of the cyclone, onto a cartridge or baghouse filter, or from both a cyclone and a cartridge or baghouse filter.

Spray Drying Using Buchi Spray Dryer. Dry powders were prepared by spray drying on a Büchi B-290 Mini Spray Dryer (BÜCHI Labortechnik AG, Flawil, Switzerland) with powder collection from either a standard or High Performance cyclone. The system was run either with air or nitrogen as the drying and atomization gas in open-loop (single pass) mode. When run using air, the system used the Büchi B-296 dehumidifier to ensure stable temperature and humidity of the air used to spray dry. Furthermore, when the relative humidity in the room exceeded 30% RH, an external LG dehumidifier (model 49007903, LG Electronics, Englewood Cliffs, NJ) was run constantly. When run using nitrogen, a pressurized source of nitrogen was used. Furthermore, the aspirator of the system was adjusted to maintain the system pressure at −2.0″ water column. Atomization of the liquid feed utilized a Büchi two-fluid nozzle with a 1.5 mm diameter or a Schlick 970-0 atomizer with a 0.5 mm liquid insert (Düsen-Schlick GmbH, Coburg, Germany). Inlet temperature of the process gas can range from 100° C. to 220° C. and outlet temperature from 30° C. to 120° C. with a liquid feedstock flowrate of 3 mL/min to 10 mL/min. The two-fluid atomizing gas ranges from 25 mm to 45 mm (300 LPH to 530 LPH) for the Büchi two-fluid nozzle and for the Schlick atomizer an atomizing air pressure of upwards of 0.3 bar. The aspirator rate ranges from 50% to 100%.

Stability Assessment: The physicochemical stability and aerosol performance of select formulations were assessed at 2-8° C. 25° C./60% RH, and when material quantities permitted, 40° C./75% RH as detailed in the International Conference on Harmonisation (ICH) Q1 guidance. Stability samples were stored in calibrated chambers (Darwin Chambers Company Models PH024 and PH074, St. Louis. MO). Bulk powder samples were weighed into amber glass vials, sealed under 30% RH, and induction-sealed in aluminum pouches (Drishield 3000, 3M, St. Paul, MN) with silica desiccant (2.0 g, Multisorb Technologies, Buffalo, NY). Additionally, to assess the stability of the formulations in capsules, the target mass of powder was weighed by hand into a size 3, HPMC capsule (Capsugel Vcaps, Peapack, NJ) with a +/−0.2 mg tolerance at 30% RH. Filled capsules were then aliquoted into high-density polyethylene (HDPE) bottles and induction sealed in aluminum pouches with silica desiccant.

### Example 1. Dry Powder Formulations of Polysorbate 80-Stabilized Nanocrystalline Itraconazole Comprising Sodium Sulfate/Mannitol

### A. Powder Preparation

The nanocrystalline itraconazole was prepared by compounding 11.662 g of itraconazole (Neuland lot ITI0114005) in 103.789 g of water and 1.1662 g of polysorbate 80 (Spectrum lot 2DI0112). 129.625 g of 500 μm polystyrene milling media (Dow Chemical, Midland MI) was then added to the suspension, and the suspension was milled at 1000 rpm for one hour then 1500 rpm for 30 minutes before being collected. The final median particle size (Dv(50)) of the milled suspension was 124 nm.

Feedstock solutions were prepared and used to manufacture dry powders composed of nanocrystalline itraconazole, polysorbate 80 and other additional excipients. Drug loads of 20 wt % and 50 wt % itraconazole, on a dry basis, were targeted. The feedstock solutions that were used to spray dry particles were made as follows. The required quantity of water was weighed into a suitably sized glass vessel. The excipients were added to the water and the solution allowed to stir until visually clear. The itraconazole-containing suspension was then added to the excipient solution and stirred until visually homogenous. The feedstocks were then spray-dried. Feedstocks were stirred while spray dried. Feedstock masses were 83.3 g, which supported manufacturing campaigns of 15 minutes. Table 2 lists the components of the feedstocks used in preparation of the dry powders.

TABLE 2
Feedstock compositions

| | | | | Sodium | | |
|---|---|---|---|---|---|---|
| | Water | Itraconazole | Polysorbate | sulfate | Mannitol | Total mass |
| Formulation | (g) | (g) | 80 (g) | (g) | (g) | (gm) |
| I | 80.8450 | 0.50027 | 0.05002 | 0.9782 | 0.9783 | 83.3543 |
| II | 80.8375 | 1.24973 | 0.12497 | 0.5672 | 0.5643 | 83.3413 |

Dry powders of Formulations I and II were manufactured from these feedstocks by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Büchi nozzle with 1.5 mm cap and 0.7 liquid tip. The aspirator of the system was adjusted to maintain the system pressure at −2.0″ water column.

The following spray drying conditions were followed to manufacture the dry powders. For Formulations I and II, the liquid feedstock solids concentration was 3.0 wt %, the process gas inlet temperature was 117° C. to 119° C., the process gas outlet temperature was 50° C., the drying gas flowrate was 17.0 kg/hr, the atomization gas flowrate was 30.4 g/min, the atomization gas, and the liquid feedstock flowrate was 6.0 mL/min. The resulting dry powders are reported in Table 3 below.

TABLE 3
Dry Powder Composition (w/w), dry basis
Dry Powder composition

| Formulation | Dry Powder Composition (w/w), dry basis |
|---|---|
| I | 20% itraconazole, 39% sodium sulfate, |
| | 39% mannitol, 2% polysorbate 80 |
| II | 50% itraconazole, 22.5% sodium sulfate, |
| | 22.5% mannitol, 5% polysorbate 80 |

### B. Powder Characterization

The bulk particle size characteristics for the two formulations are found in Table 4. The span at 1 bar of 1.83 and 1.67 for Formulations I and II, respectively, indicates a relatively narrow size distribution. The 1 bar/4 bar dispersibility ratio of 1.07 and 1.12 for Formulations I and II respectively, indicate that they are relatively independent of dispersion energy, a desirable characteristic which allows similar particle dispersion across a range of dispersion energies.

TABLE 4
Bulk particle size

| | 0.5 bar | | 1 bar | | 4 bar | | |
|---|---|---|---|---|---|---|---|
| | Dv[50] | | Dv[50] | | Dv[50] | | 1 bar:4 bar |
| Formulation | (μm) | Span | (μm) | Span | (μm) | Span | Dv[50] ratio |
| I | 2.28 | 1.65 | 1.89 | 1.83 | 1.77 | 1.93 | 1.07 |
| II | 2.42 | 1.64 | 2.05 | 1.67 | 1.84 | 1.83 | 1.12 |

The geometric particle size and capsule emitted powder mass (CEPM) measured and/or calculated at 60 liters per minute (LPM) and 20 LPM simulated patient flow rates were measured for the two formulations and reported in Table 5. The small changes in CEPM and geometric size from 60 LPM to 20 LPM indicates that the dry powders are relatively independent of patient inspiratory flowrate, indicating that patients breathing in at varying flow rates would receive a relatively similar therapeutic dose.

TABLE 5
Emitted particle size

| | |
|---|---|
| 20 LPM | 60 LPM |

| | CEPM | Dv[50] | CEPM | Dv[50] |
|---|---|---|---|---|
| Formulation | (%) | (μm) | (%) | (μm) |
| I | 89.6 | 4.62 | 100.4 | 2.46 |
| II | 42.5 | 8.82 | 97.4 | 2.31 |

The aerodynamic particle size, fine particle fractions and fine particle doses measured and/or calculated with an eight-stage Anderson Cascade Impactor (ACI-8) are reported in Table 6. The fine particle dose for Formulations I and II both indicate a high percentage of the nominal dose which is filled into the capsule reaches the impactor stages (38.8% and 37.1%, respectively) and so would be predicted to be delivered to the lungs. The MMAD of Formulations I and II were 3.59 microns and 3.17 microns, respectively, indicating deposition in the central and conducting airways.

TABLE 6
Aerodynamic particle size

| | MMAD | FPD <5 μm |
|---|---|---|
| Formulation | (μm) | (% nominal dose) |
| I | 3.59 | 38.8 |
| II | 3.17 | 37.1 |

The weight loss of Formulations I and II were measured via TGA and were found to be 0.48% and 0.15%, respectively.

The itraconazole content of Formulations I and II were measured with HPLC-UV and are 102.9% and 103.1%, respectively.

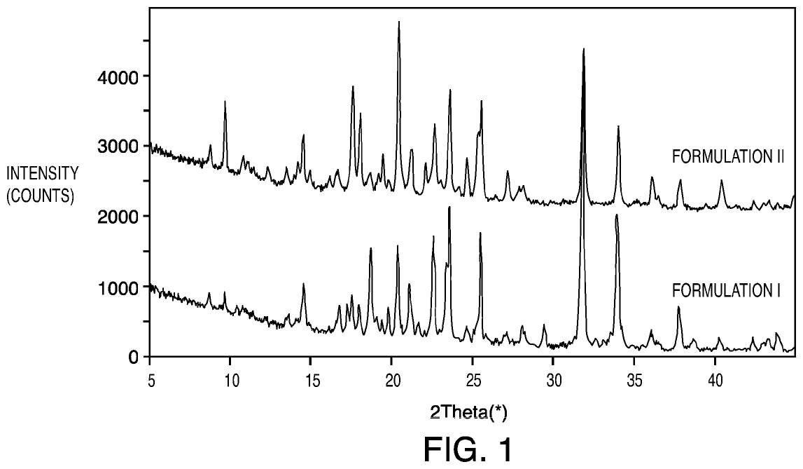

The crystallinity of Formulations I and II were assessed via XRD. The diffraction pattern of itraconazole is observed in both formulations, suggesting the milling or spray drying process does not affect the solid-state of itraconazole. Additional peaks observed in the patterns correspond to the additional excipients in the formulations. (FIG. 1)

Formulations I and II were determined to be stable after being stored for six months at 2-8° C. and 25° C./60% RH.

### Example 2. Dry Powder Formulations of Polysorbate 80-Stabilized Nanocrystalline Itraconazole Comprising Sodium Chloride/Leucine

### A. Powder Preparation

The nanocrystalline itraconazole was prepared by compounding 11.662 g of itraconazole (Neuland lot ITI0114005) in 103.789 g of water and 1.1662 g of polysorbate 80 (Spectrum lot 2DI0112). 129.625 g of 500 μm polystyrene milling media (Dow Chemical, Midland MI) was then added to the suspension, and the suspension was milled at 1000 rpm for one hour then 1500 rpm for 30 minutes before being collected. The final median particle size (Dv(50)) of the milled suspension was 124 nm.

Feedstock solutions were prepared and used to manufacture dry powders composed of nanocrystalline itraconazole, polysorbate 80 and other additional excipients. Drug loads of 20 wt % and 50 wt % itraconazole, on a dry basis, were targeted. The feedstock solutions that were used to spray dry particles were made as follows. The required quantity of water was weighed into a suitably sized glass vessel. The excipients were added to the water and the solution allowed to stir until visually clear. The itraconazole-containing suspension was then added to the excipient solution and stirred until visually homogenous. The feedstocks were then spray-dried. Feedstocks were stirred while spray dried. Feedstock masses were 83.3 g, which supported manufacturing campaigns of 15 minutes. Table 7 lists the components of the feedstocks used in preparation of the dry powders.

TABLE 7
Feedstock compositions

| | | | | Sodium | | |
|---|---|---|---|---|---|---|
| | Water | Itraconazole | Polysorbate | chloride | Leucine | Total mass |
| Formulation | (g) | (g) | 80 (g) | (g) | (g) | (gm) |
| III | 80.806043 | 0.49887 | 0.0489887 | 1.5548 | 0.3881 | 83.2977 |
| IV | 80.829235 | 1.24915 | 0.124915 | 0.8956 | 0.2210 | 83.3199 |

Dry powders of Formulations I and II were manufactured from these feedstocks by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Büchi nozzle with 1.5 mm cap and 0.7 liquid tip. The aspirator of the system was adjusted to maintain the system pressure at −2.0″ water column.

The following spray drying conditions were followed to manufacture the dry powders. For Formulations I and II, the liquid feedstock solids concentration was 3.0%, the process gas inlet temperature was 138° C. to 141° C., the process gas outlet temperature was 60° C., the drying gas flowrate was 17.0 kg/hr, the atomization gas flowrate was 30.4 g/min, the atomization gas, and the liquid feedstock flowrate was 6.0 mL/min. The resulting dry powders are reported in Table 8.

TABLE 8
Dry powder compositions, dry basis

| Formulation | Dry Powder Composition (w/w), dry basis |
|---|---|
| III | 20% itraconazole, 62.4% sodium chloride, |
| | 15.6% leucine, 2% polysorbate 80 |
| IV | 50% itraconazole, 36% sodium chloride, |
| | 9% leucine, 5% polysorbate 80 |

### B. Powder Characterization

The bulk particle size characteristics for the two formulations are found in Table 9. The span at 1 bar of 1.76 and 1.86 for Formulations III and IV, respectively, indicates a relatively narrow size distribution. The 1 bar/4 bar dispersibility ratio of 1.19 and 1.05 for Formulations III and IV respectively, indicate that they are relatively independent of dispersion energy, a desirable characteristic which allows similar particle dispersion across a range of dispersion energies.

TABLE 9
Bulk particle size

| | 0.5 bar | | 1 bar | | 4 bar | | |
|---|---|---|---|---|---|---|---|
| | Dv[50] | | Dv[50] | | Dv[50] | | 1 bar:4 bar |
| Formulation | (μm) | Span | (μm) | Span | (μm) | Span | Dv[50] ratio |
| III | 2.13 | 1.81 | 1.94 | 1.76 | 1.63 | 1.67 | 1.19 |
| IV | 2.07 | 1.81 | 1.93 | 1.86 | 1.85 | 1.81 | 1.05 |

The geometric particle size and capsule emitted powder mass (CEPM) measured and/or calculated at 60 liters per minute (LPM) and 20 LPM simulated patient flow rates were measured for the two formulations and reported in Table 10. The small changes in CEPM and geometric size from 60 LPM to 20 LPM indicates that the dry powders are relatively independent of patient inspiratory flowrate, indicating that patients breathing in at varying flow rates would receive a relatively similar therapeutic dose.

TABLE 10
Emitted particle size

| | |
|---|---|
| 20 LPM | 60 LPM |

| | CEPM | Dv[50] | CEPM | Dv[50] |
|---|---|---|---|---|
| Formulation | (%) | (μm) | (%) | (μm) |
| III | 98.5 | 3.77 | 99.5 | 2.10 |
| IV | 60.6 | 4.75 | 100.1 | 2.31 |

The aerodynamic particle size, fine particle fractions and fine particle doses measured and/or calculated with an eight-stage Anderson Cascade Impactor (ACI-8) are reported in Table 11. The fine particle dose for Formulation III and IV both indicate a high percentage of the nominal dose which is filled into the capsule reaches the impactor stages (55.5% and 49.4%, respectively) and so would be predicted to be delivered to the lungs. The MMAD of Formulation III and IV were 3.14 microns and 3.30 microns, respectively, indicating deposition in the central and conducting airways.

TABLE 11
Aerodynamic particle size

| | MMAD | FPD <5 μm |
|---|---|---|
| Formulation | (μm) | (% nominal dose) |
| III | 3.14 | 55.5 |
| IV | 3.30 | 49.4 |

The weight loss of Formulations III and IV were measured via TGA and were found to be 0.15% and 0.08%, respectively.

The itraconazole content of Formulations III and IV were measured with HPLC-UV and are 103.7% and 104.9%, respectively.

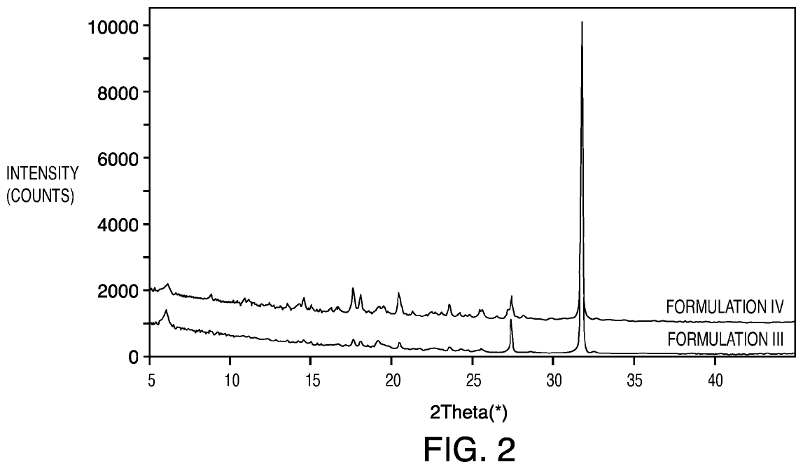

The crystallinity of Formulations III and IV were assessed via XRD. The diffraction pattern of itraconazole is observed in both formulations, suggesting the milling or spray drying process does not affect the solid-state of itraconazole. Additional peaks observed in the patterns correspond to the additional excipients in the formulations. (FIG. 2)

Formulations III and IV were determined to be stable after being stored for six months at 2-8° C. and 25° C./60% RH.

### Example 3. Dry Powder Formulations of Polysorbate 80-Stabilized Nanocrystalline Itraconazole Comprising Magnesium Lactate/Leucine

### A. Powder Preparation

The nanocrystalline itraconazole was prepared by compounding 11.662 g of itraconazole (Neuland lot ITI0114005) in 103.789 g of water and 1.1662 g of polysorbate 80 (Spectrum lot 2DI0112). 129.625 g of 500 μm polystyrene milling media (Dow Chemical, Midland MI) was then added to the suspension, and the suspension was milled at 1000 rpm for one hour then 1500 rpm for 30 minutes before being collected. The final median particle size (Dv(50)) of the milled suspension was 124 nm.

Feedstock solutions were prepared and used to manufacture dry powders composed of nanocrystalline itraconazole, polysorbate 80 and other additional excipients. Drug loads of 20 wt % and 50 wt % itraconazole, on a dry basis, were targeted. The feedstock solutions that were used to spray dry particles were made as follows. The required quantity of water was weighed into a suitably sized glass vessel. The excipients were added to the water and the solution allowed to stir until visually clear. The itraconazole-containing suspension was then added to the excipient solution and stirred until visually homogenous. The feedstocks were then spray-dried. Feedstocks were stirred while spray dried. Feedstock masses were 83.3 g, which supported manufacturing campaigns of 15 minutes. Table 12 lists the components of the feedstocks used in preparation of the dry powders.

TABLE 12
Feedstock compositions

| | | | | Magnesium | | |
|---|---|---|---|---|---|---|
| | Water | Itraconazole | Polysorbate | lactate | Leucine | Total mass |
| Formulation | (g) | (g) | 80 (g) | (g) | (g) | (gm) |
| V | 80.83964 | 0.5006 | 0.05006 | 2.1096 | 0.2923 | 83.7922 |
| VI | 80.83467 | 1.25021 | 0.125021 | 1.2177 | 0.1718 | 83.5994 |

Dry powders of Formulations V and VI were manufactured from these feedstocks by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Buchi nozzle with 1.5 mm cap and 0.7 liquid tip. The aspirator of the system was adjusted to maintain the system pressure at −2.0″ water column.

The following spray drying conditions were followed to manufacture the dry powders. For Formulations V and VI, the liquid feedstock solids concentration was 3.0%, the process gas inlet temperature was 171° C. to 173° C., the process gas outlet temperature was 80° C., the drying gas flowrate was 17.0 kg/hr, the atomization gas flowrate was 30.4 g/min, the atomization gas, and the liquid feedstock flowrate was 6.0 mL/min. The resulting dry powders are reported in Table 13.

TABLE 13
Dry powder compositions, dry basis

| Formulation | Dry Powder Composition (w/w), dry basis |
|---|---|
| V | 20% itraconazole, 66.3% magnesium lactate, |
| | 11.7% leucine, 2% polysorbate 80 |
| VI | 50% itraconazole, 38.25% magnesium lactate, |
| | 6.75% leucine, 5% polysorbate 80 |

### B. Powder Characterization

The bulk particle size characteristics for the two formulations are found in Table 14. The span at 1 bar of 1.70 and 1.83 for Formulations V and VI, respectively, indicates a relatively narrow size distribution. The 1 bar/4 bar dispersibility ratio of 1.02 and 1.05 for Formulations V and VI respectively, indicate that they are relatively independent of dispersion energy, a desirable characteristic which allows similar particle dispersion across a range of dispersion energies.

TABLE 14
Bulk particle size

| | 0.5 bar | | 1 bar | | 4 bar | | |
|---|---|---|---|---|---|---|---|
| | Dv[50] | | Dv[50] | | Dv[50] | | 1 bar:4 bar |
| Formulation | (μm) | Span | (μm) | Span | (μm) | Span | Dv[50] ratio |
| V | 2.75 | 1.64 | 2.60 | 1.70 | 2.55 | 1.67 | 1.02 |
| VI | 2.36 | 1.76 | 2.14 | 1.83 | 2.04 | 1.88 | 1.05 |

The geometric particle size and capsule emitted powder mass (CEPM) measured and/or calculated at 60 liters per minute (LPM) and 20 LPM simulated patient flow rates were measured for the two formulations and reported in Table 15. The small changes in CEPM and geometric size from 60 LPM to 20 LPM indicates that the dry powders are relatively independent of patient inspiratory flowrate, indicating that patients breathing in at varying flow rates would receive a relatively similar therapeutic dose.

TABLE 15
Emitted particle size

| | |
|---|---|
| 20 LPM | 60 LPM |

| | CEPM | Dv[50] | CEPM | Dv[50] |
|---|---|---|---|---|
| Formulation | (%) | (μm) | (%) | (μm) |
| V | 95.2 | 5.21 | 98.5 | 2.77 |
| VI | 93.5 | 3.93 | 98.1 | 2.34 |

The aerodynamic particle size, fine particle fractions and fine particle doses measured and/or calculated with an eight-stage Anderson Cascade Impactor (ACI-8) are reported in Table 16. The fine particle dose for Formulations V and VI both indicate a high percentage of the nominal dose which is filled into the capsule reaches the impactor stages (39.6% and 44.6%, respectively) and so would be predicted to be delivered to the lungs. The MMAD of Formulations V and VI were 3.97 microns and 3.42 microns, respectively, indicating deposition in the central and conducting airways.

TABLE 16
Aerodynamic particle size

| | MMAD | FPD <5 μm |
|---|---|---|
| Formulation | (μm) | (% nominal dose) |
| V | 3.97 | 39.6 |
| VI | 3.42 | 44.6 |

The weight loss of Formulations V and VI were measured via TGA and were found to be 5.157% and 3.087%, respectively.

The itraconazole content of Formulations V and VI were measured with HPLC-UV and are 99.7% and 100.6%, respectively.

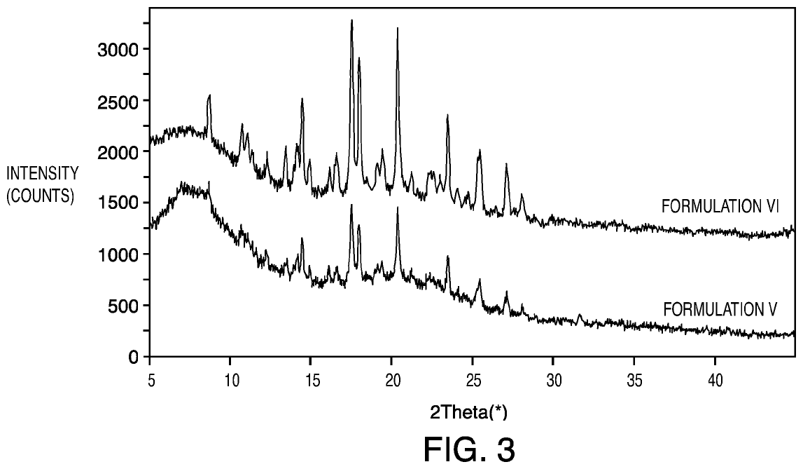

The crystallinity of Formulations V and VI were assessed via XRD. The diffraction pattern of itraconazole is observed in both formulations, suggesting the milling or spray drying process does not affect the solid-state of itraconazole. Additional peaks observed in the patterns correspond to the additional excipients in the formulations. (FIG. 3)

Formulations V and VI were determined to be stable after being stored for six months at 2-8° C. and 25° C./60% RH.

### Example 4. Dry Powder Formulations of Oleic Acid-Stabilized Nanocrystalline Itraconazole Comprising Sodium Sulfate/Leucine

### A. Powder Preparation

The nanocrystalline itraconazole was prepared by compounding 11.646 g of itraconazole (Neuland lot ITI0114005) in 104.233 g of water, 0.582 g of oleic acid (Croda 000705097), and 9.44 g of 10% ammonium hydroxide. 129.625 g of 500 μm polystyrene milling media (Dow Chemical, Midland MI) was then added to the suspension, and the suspension was milled at 1000 rpm for one hour and then 1500 rpm for an additional hour before being collected. The final median particle size (Dv(50)) of the milled suspension was 120 nm.

Feedstock solutions were prepared and used to manufacture dry powders composed of nanocrystalline itraconazole, oleic acid and other additional excipients. Drug loads of 50 wt % and 70 wt % itraconazole, on a dry basis, were targeted. The feedstock solutions that were used to spray dry particles were made as follows. The required quantity of water was weighed into a suitably sized glass vessel. The excipients were added to the water and the solution allowed to stir until visually clear. The itraconazole-containing suspension was then added to the excipient solution and stirred until visually homogenous. The feedstocks were then spray-dried. The feedstocks were stirred while spray dried. Feedstock volumes ranged from 100 to 193.3 g, which supported manufacturing campaigns from 16 to 34 minutes. Table 17 lists the components of the feedstocks used in preparation of the dry powders.

TABLE 17
Feedstock compositions

| | | | Oleic | Sodium | | Ammonium | Total |
|---|---|---|---|---|---|---|---|
| | Water | Itraconazole | acid | sulfate | Leucine | hydroxide | mass |
| Formulation | (g) | (g) | (g) | (g) | (g) | (g) | (gm) |
| VII | VII | 187.5173 | 2.6775 | 0.1338 | 1.9288 | 0.8267 | 0.2170 |
| VIII | VIII | 96.98351 | 1.93610 | 0.0968 | 0.3988 | 0.3971 | 0.1569 |

Dry powders of Formulations VII and VIII were manufactured from these feedstocks by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Büchi nozzle with 1.5 mm cap and 0.7 liquid tip. The aspirator of the system was adjusted to maintain the system pressure at −2.0″ water column.

The following spray drying conditions were followed to manufacture the dry powders. For Formulations VII and VIII, the liquid feedstock solids concentration was 3.0%, the process gas inlet temperature was 131° C. to 133° C., the process gas outlet temperature was 60° C., the drying gas flowrate was 17.0 kg/hr, the atomization gas flowrate was 30.4 g/min (1.824 kg/hr), and the liquid feedstock flowrate was 6.0 mL/min. The resulting dry powders are reported in Table 18.

TABLE 18
Dry powder compositions, dry basis

| Formulation | Dry Powder Composition (w/w), dry basis |
|---|---|
| VII | 50% itraconazole, 33.25% sodium sulfate, |
| | 14.25% leucine, 2.5% oleic acid |
| VIII | 70% itraconazole, 13.25% sodium sulfate, |
| | 13.25% leucine, 3.5% oleic acid |

### B. Powder Characterization

The bulk particle size characteristics for the two formulations are found in Table 19. The span at 1 bar of 1.94 and 1.81 for Formulations VII and VIII, respectively, indicates a relatively narrow size distribution. The 1 bar/4 bar dispersibility ratio of 1.22 and 1.11 for Formulations VII and VIII respectively, indicate that they are relatively independent of dispersion energy, a desirable characteristic which allows similar particle dispersion across a range of dispersion energies.

TABLE 19
Bulk particle size

| | 0.5 bar | | 1 bar | | 4 bar | | |
|---|---|---|---|---|---|---|---|
| | Dv[50] | | Dv[50] | | Dv[50] | | 1 bar:4 bar |
| Formulation | (μm) | Span | (μm) | Span | (μm) | Span | Dv[50] ratio |
| VII | 2.44 | 1.88 | 2.22 | 1.94 | 1.81 | 1.91 | 1.22 |
| VIII | 2.77 | 1.72 | 2.50 | 1.81 | 2.26 | 1.96 | 1.11 |

The geometric particle size and capsule emitted powder mass (CEPM) measured and/or calculated at 60 liters per minute (LPM) and 20 LPM simulated patient flow rates were measured for the two formulations and reported in Table 20. The small changes in CEPM and geometric size from 60 LPM to 20 LPM indicates that the dry powders are relatively independent of patient inspiratory flowrate, indicating that patients breathing in at varying flow rates would receive a relatively similar therapeutic dose.

TABLE 20
Emitted particle size

| | |
|---|---|
| 20 LPM | 60 LPM |

| | CEPM | Dv[50] | CEPM | Dv[50] |
|---|---|---|---|---|
| Formulation | (%) | (μm) | (%) | (μm) |
| VII | 97.2 | 3.08 | 97.6 | 2.36 |
| VIII | 95.6 | 3.21 | 98.2 | 2.68 |

The aerodynamic particle size, fine particle fractions and fine particle doses measured and/or calculated with an eight-stage Anderson Cascade Impactor (ACI-8) are reported in Table 21. The fine particle dose for Formulation VII and VIII both indicate a high percentage of the nominal dose which is filled into the capsule reaches the impactor stages (56.0% and 52.6%, respectively) and so would be predicted to be delivered to the lungs. The MMAD of Formulation VII and VIII were 2.77 microns and 3.08 microns, respectively, indicating deposition in the central and conducting airways.

TABLE 21
Aerodynamic particle size

| | MMAD | FPD <5 μm |
|---|---|---|
| Formulation | (μm) | (% nominal dose) |
| VII | 2.77 | 56.0 |
| VIII | 3.08 | 52.6 |

The weight loss of Formulations VII and VIII were measured via TGA and were found to be 0.47% and 0.33%, respectively.

The itraconazole content of Formulations VII and VIII were measured with HPLC-UV and are 101.5% and 101.4%, respectively.

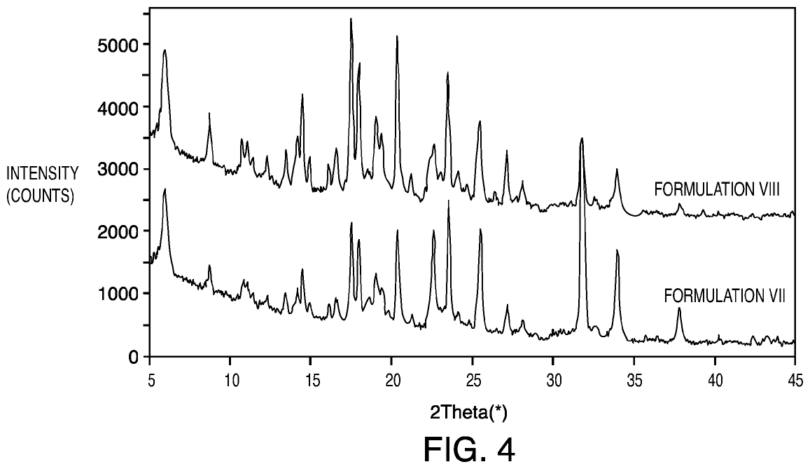

The crystallinity of Formulations VII and VIII were assessed via XRD. The diffraction pattern of itraconazole is observed in both formulations, suggesting the milling or spray drying process does not affect the solid-state of itraconazole. Additional peaks observed in the patterns correspond to the additional excipients in the formulations. (FIG. 4)

Formulations VII and VIII were determined to be stable after being stored for six months at 2-8° C. and 25° C./60% RH.

### Example 5. Reference Liquid Nanocrystalline and Microcrystalline Itraconazole Formulations

Liquid formulations of crystalline particulate itraconazole were prepared.

Formulation IX is a micro-suspension of itraconazole with polysorbate 80. The itraconazole concentration in the liquid is 5 mg/mL. The ratio of itraconazole to polysorbate 80 is 10:1 (wgt/wgt). The median size of the itraconazole crystals is 1600 nanometers.

Formulation X is a nano-suspension of itraconazole with polysorbate 80. The itraconazole concentration in the liquid is 5 mg/mL. The ratio of itraconazole to polysorbate 80 is 10:1 (wgt/wgt). The median size of the itraconazole crystals is 132 nanometers.

### Example 6. Dry Powder Formulation of Oleic Acid-Stabilized Nanocrystalline Itraconazole Comprising Sodium Sulfate/Leucine

### A. Powder Preparation

The nanocrystalline itraconazole was prepared by compounding 30.374 g of itraconazole (Neuland ITI0714011) in 87.018 g of water, 1.519 g of oleic acid (Croda 000705097), and 2.585 g ammonium hydroxide (Acros B0522464). 129.625 g of 500 μm polystyrene milling media (Dow Chemical, Midland MI) was then added to the suspension, and the suspension was milled at 1800 rpm for two hours before being collected. The final median particle size (Dv(50)) of the milled suspension was 124 nm.

A feedstock solution was prepared and used to manufacture a dry powder composed of nanocrystalline itraconazole, oleic acid and other additional excipients. A drug load of 50 wt % itraconazole, on a dry basis, was targeted. The feedstock solution that was used to spray dry particles were made as follows. The required quantity of water was weighed into a suitably sized glass vessel. The excipients were added to the water and the solution allowed to stir until visually clear. The itraconazole-containing suspension was then added to the excipient solution and stirred until visually homogenous. The feedstock was then spray-dried. Feedstock mass was 1219.4 g, which supported a manufacturing campaign of approximately 3.5 hours. Table 22 lists the components of the feedstock used in preparation of the dry powder.

TABLE 22
Feedstock composition

| | | | Oleic | Sodium | | Ammonium | Total |
|---|---|---|---|---|---|---|---|
| | Water | Itraconazole | acid | sulfate | Leucine | hydroxide | mass |
| Formulation | (g) | (g) | (g) | (g) | (g) | (g) | (gm) |
| XI | 1181.9273 | 18.3 | 0.915 | 12.8071 | 4.517 | 1.5577 | 1220.08 |

A dry powder of Formulation XI was manufactured from this feedstock by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Büchi nozzle with 1.5 mm cap and 0.7 liquid tip. The aspirator of the system was adjusted to maintain the system pressure at −2.0″ water column.

The following spray drying conditions were followed to manufacture the dry powder. For Formulations XI, the liquid feedstock solids concentration was 3.0%, the process gas inlet temperature was 129° C. to 132° C., the process gas outlet temperature was 60° C., the drying gas flowrate was 17.0 kg/hr, the atomization gas flowrate was 30.4 g/min, and the liquid feedstock flowrate was 6.0 mL/min. The resulting dry powder is reported in Table 23.

TABLE 23
Dry powder composition, dry basis

| Formulation | Dry Powder Composition (w/w), dry basis |
|---|---|
| XI | 50% itraconazole, 35% sodium sulfate, |
| | 12.5% leucine, 2.5% oleic acid |

### B. Powder Characterization

The bulk particle size characteristics for the formulation are found in Table 24. The span at 1 bar of 2.77 for Formulations XI, indicates a relatively narrow size distribution. The 1 bar/4 bar dispersibility ratio of 1.28 for Formulations XI, indicates the particle size is relatively independent of dispersion energy, a desirable characteristic which allows similar dispersion across a range of dispersion energies.

TABLE 24
Bulk particle size

| | 0.5 bar | | 1 bar | | 4 bar | | |
|---|---|---|---|---|---|---|---|
| | Dv[50] | | Dv[50] | | Dv[50] | | 1 bar:4 bar |
| Formulation | (μm) | Span | (μm) | Span | (μm) | Span | Dv[50] ratio |
| XI | 2.96 | 2.53 | 2.46 | 2.77 | 1.92 | 2.48 | 1.28 |

The geometric particle size and capsule emitted powder mass (CEPM) measured and/or calculated at 60 liters per minute (LPM) and 20 LPM simulated patient flow rates were measured for the formulation and reported in Table 25. The small changes in CEPM and geometric size from 60 LPM to 20 LPM indicates that the dry powder is relatively independent of patient inspiratory flowrate, indicating that patients breathing in at varying flow rates would receive a relatively similar therapeutic dose.

TABLE 25
Emitted particle size

| | |
|---|---|
| 20 LPM | 60 LPM |

| | CEPM | Dv[50] | CEPM | Dv[50] |
|---|---|---|---|---|
| Formulation | (%) | (μm) | (%) | (μm) |
| XI | 98.6 | 4.37 | 99.1 | 3.62 |

The aerodynamic particle size, fine particle fractions and fine particle doses measured and/or calculated with a Next Generation Impactor (NGI) are reported in Table 26. The fine particle dose for Formulation XI indicates a high percentage of the nominal dose which is filled into the capsule reaches the impactor stages (42%) and so would be predicted to be delivered to the lungs. The MMAD of Formulation XI was 3.37 microns, indicating deposition in the central and conducting airways.

TABLE 26
Aerodynamic particle size

| | MMAD | FPD <5 μm |
|---|---|---|
| Formulation | (μm) | (% nominal dose) |
| XI | 3.37 | 42.0 |

The weight loss of Formulation XI was measured via TGA and was found to be 0.31%.

The itraconazole content of Formulation XI was measured with HPLC-UV and is 99.7%.

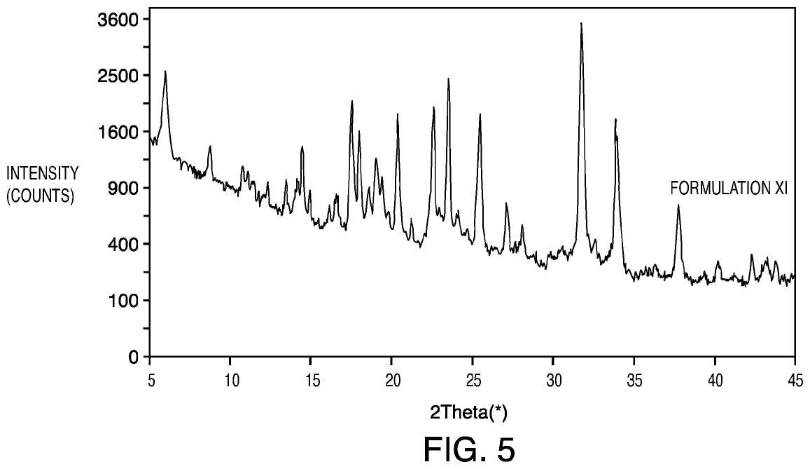

The crystallinity of Formulation XI was assessed via XRD. The diffraction pattern of itraconazole is observed in the formulation, suggesting the milling or spray drying process does not affect the solid-state of itraconazole. Additional peaks observed in the pattern correspond to the additional excipients in the formulations. (FIG. 5)

### Example 7. Dry Powder Formulations of Polysorbate 80-Stabilized Crystalline Itraconazole of Varying Particle Sizes Comprising Sodium Sulfate/Leucine

### A. Powder Preparation

The nanocrystalline itraconazole for Formulation XII was prepared by compounding 30.090 g of itraconazole (Neuland ITI0114005 and ITI0714011) in 87.262 g of water and 3.009 g of polysorbate 80. 129.625 g of 500 μm polystyrene milling media (Dow Chemical, Midland MI) was then added to the suspension, and the suspension was milled at 1800 rpm for one hour before being collected. The final median particle size (Dv (50)) of the milled suspension was 132 nm. This process is called the “Wet milling process #1”, hereafter.

The nanocrystalline itraconazole for Formulation XIII was prepared as a suspension comprising 10 wt % itraconazole and 1.0 wt % polysorbate 80 in deionized water. The polysorbate 80 was dissolved in 89.0% DI water via magnetic stir bar, then the itraconazole was slowly added also with a magnetic stir bar. Once all of the itraconazole was suspended the formulation was processed on the M-110P Microfluidizer processor at 30,000 psi for 120 passes using an ice water cooling coil to cool the material during processing. The final median particle size (Dv(50)) of the milled suspension was 198 nm. This process is called the “Microfluidics process #1”, hereafter.

The nanocrystalline itraconazole for Formulation XIV was prepared by compounding 30.090 g of itraconazole (Neuland ITI0114005) in 87.26195 g of water and 3.009 g of polysorbate 80. 129.625 g of 500 μm polystyrene milling media (Dow Chemical, Midland MI) was then added to the suspension, and the suspension was milled at 1000 rpm for 30 minutes before being collected. The final median particle size (Dv(50)) of the milled suspension was 258 nm. This process is called the “Wet milling process #2”, hereafter.

The microcrystalline itraconazole for Formulation XV was prepared using a Qualification Micronizer jet mill (Sturtevant, Hanover, MA USA). The feed pressure was set to 90 psig and the grind pressure was set to 40 psig. Itraconazole was continuously fed into the mill until 60.3 g of itraconazole was milled. The final median particle size (Dv(50)) of the milled API was 1600 nm. This process is called the “Jet milling process #1”, hereafter. The micronized itraconazole for Formulation XV was then compounded into a suspension consisting of 10 wt % itraconazole and 1.0 wt % polysorbate 80 in deionized water. The batch size was 200 g. The polysorbate 80 was dissolved in 89.0% DI water via magnetic stir bar, then the itraconazole was slowly added and allowed to mix until the suspension was observed to be visually dispersed and homogeneous.

Feedstock solutions were prepared and used to manufacture dry powders composed of crystalline itraconazole, polysorbate 80 and other additional excipients. A drug load of 50 wt % itraconazole, on a dry basis, was targeted. The feedstock solutions that were used to spray dry particles were made as follows. The required quantity of water was weighed into a suitably sized glass vessel. The excipients were added to the water and the solution allowed to stir until visually clear. The itraconazole-containing suspension was then added to the excipient solution and stirred until visually homogenous. The feedstocks were then spray-dried. Feedstocks were stirred while spray dried. Feedstock masses were 166.67 g to 1219.4 g, which supported manufacturing campaigns of 30 minutes to 3.5 hours. Table 27 lists the components of the feedstocks used in preparation of the dry powders.

TABLE 27
Feedstock compositions

| | | | | Sodium | | |
|---|---|---|---|---|---|---|
| | Water | Itraconazole | Polysorbate | sulfate | Leucine | Total mass |
| Formulation | (g) | (g) | 80 (g) | (g) | (g) | (gm) |
| XII | 1183.17 | 18.3 | 1.83 | 12.8009 | 3.662 | 1219.7629 |
| XIII | 161.654 | 2.501 | 0.250 | 1.57553 | 0.67525 | 166.6555 |
| XIV | 1080.13 | 16.70 | 1.670 | 11.69625 | 3.34343 | 1113.5397 |
| XV | 1079.619 | 16.71 | 1.671 | 11.69165 | 3.34353 | 1113.035 |

Dry powders of Formulations XII-XV were manufactured from these feedstocks by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Büchi nozzle with 1.5 mm cap and 0.7 liquid tip. The aspirator of the system was adjusted to maintain the system pressure at −2.0″ water column.

The following spray drying conditions were followed to manufacture the dry powder. For Formulations XII, XIV, and XV, the liquid feedstock solids concentration was 3%, the process gas inlet temperature was 127° C. to 140° C., the process gas outlet temperature was 60° C., the drying gas flowrate was 17.0 kg/hr, the atomization gas flowrate was 30.0 g/min, and the liquid feedstock flowrate was 6.0 mL/min. The resulting dry powders are reported in Table 28.

The following spray drying conditions were followed to manufacture the dry powder. For Formulation XIII, the liquid feedstock solids concentration was 3%, the process gas inlet temperature was 134° C. the process gas outlet temperature was 60° C. the drying gas flowrate was 17.0 kg/hr, the atomization gas flowrate was 30.4 g/min, and the liquid feedstock flowrate was 6.0 mL/min. The resulting dry powders are reported in Table 28.

TABLE 28
Dry powder composition, dry basis

| | | Dry Powder Composition (w/w), |
|---|---|---|
| Formulation | Description | dry basis |
| XII | Wet milling | 50% itraconazole, 35% sodium sulfate, |
| | process #1 | 10% leucine, 5% polysorbate 80 |
| XIII | Microfluidics | 50% itraconazole, 35% sodium sulfate, |
| | process #1 | 10% leucine, 5% polysorbate 80 |
| XIV | Wet milling | 50% itraconazole, 35% sodium sulfate, |
| | process #2 | 10% leucine, 5% polysorbate 80 |
| XV | Jet milling | 50% itraconazole, 35% sodium sulfate, |
| | process #1 | 10% leucine, 5% polysorbate 80 |

### B. Powder Characterization

The bulk particle size characteristics for the four formulations are found in Table 29. The span at 1 bar of less than 2.10 for Formulations XII-XV indicates a relatively narrow size distribution. The 1 bar/4 bar dispersibility ratio less than 1.25 for Formulations XII-XV indicate that they are relatively independent of dispersion energy, a desirable characteristic which allows similar particle dispersion across a range of dispersion energies.

TABLE 29
Bulk particle size

| | 0.5 bar | | 1 bar | | 4 bar | | |
|---|---|---|---|---|---|---|---|
| | Dv[50] | | Dv[50] | | Dv[50] | | 1 bar:4 bar |
| Formulation | (μm) | Span | (μm) | Span | (μm) | Span | Dv[50] ratio |
| XII | 3.61 | 2.01 | 3.46 | 2.08 | 3.05 | 2.15 | 1.14 |
| XIII | 2.78 | 1.73 | 2.56 | 1.90 | 2.43 | 1.86 | 1.05 |
| XIV | 4.11 | 2.03 | 3.94 | 2.04 | 3.23 | 2.21 | 1.22 |
| XV | 3.61 | 2.02 | 3.44 | 2.06 | 2.96 | 2.10 | 1.16 |

The geometric particle size and capsule emitted powder mass (CEPM) measured and/or calculated at 60 liters per minute (LPM) and 30 LPM simulated patient flow rates were measured for Formulations XII, XIV, and XV and reported in Table 30. The small changes in CEPM and geometric size from 60 LPM to 30 LPM indicates that the dry powders are relatively independent of patient inspiratory flowrate, indicating that patients breathing in at varying flow rates would receive a relatively similar therapeutic dose. Emitted particle size testing was not performed for Formulation XIII due to lack of sufficient material quantities.

TABLE 30
Emitted particle size

| | |
|---|---|
| 30 LPM | 60 LPM |

| | CEPM | Dv[50] | CEPM | Dv[50] |
|---|---|---|---|---|
| Formulation | (%) | (μm) | (%) | (μm) |
| XII | 99.3 | 4.35 | 99.8 | 3.97 |
| XIII | NT | NT | NT | NT |
| XIV | 99.2 | 5.37 | 99.8 | 4.98 |
| XV | 99.1 | 4.82 | 99.6 | 4.34 |

NT = Not tested

The aerodynamic particle size, fine particle fractions and fine particle doses measured and/or calculated with an eight-stage Anderson Cascade Impactor (ACI-8) or Next Generation Impactor (NGI) are reported in Table 31. The fine particle dose for Formulation XII through XV all indicate that greater than 30% of the nominal dose reaches the impactor stages and so would be predicted to be delivered to the lungs. The MMAD of Formulation XII through XV range from 3.42 to 4.76, indicating deposition in the central and conducting airways.

TABLE 31
Aerodynamic particle size

| | Test | MMAD | FPD <5 μm |
|---|---|---|---|
| Formulation | Method | (μm) | (% nominal dose) |
| XII | NGI | 4.22 | 38.3 |
| XIII | NGI | 3.42 | 48.2 |
| XIV | ACI-8 | 4.73 | 30.6 |
| XV | NGI | 4.76 | 33.5 |

The weight loss of Formulations VII and VIII were measured via TGA and are detailed in Table 32.

TABLE 32
Weight loss (%) via TGA

| Formulation | Weight loss via TGA (%) |
|---|---|
| XII | 0.10 |
| XIII | 0.18 |
| XIV | 0.08 |
| XV | 0.06 |

The itraconazole content of Formulations VII and VIII were measured with HPLC-UV and are detailed in Table 33.

TABLE 33
Itraconazole content

| | Itraconazole content |
|---|---|
| Formulation | (% label claim) |
| XII | 100.4 |
| XIII | 101.5 |
| XIV | 101.1 |
| XV | 97.9 |

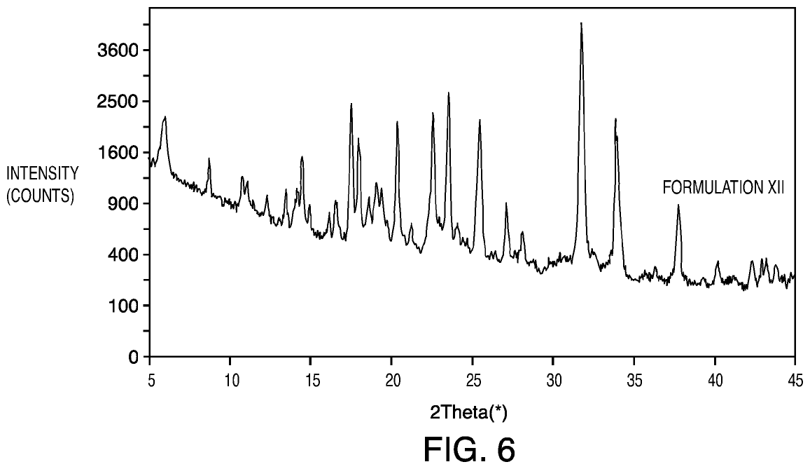

The crystallinity of Formulation XII was assessed via XRD. The diffraction pattern of itraconazole is observed in the formulation, suggesting the milling or spray drying process does not affect the solid-state of itraconazole. Additional peaks observed in the pattern correspond to the additional excipients in the formulations. (FIG. 6)

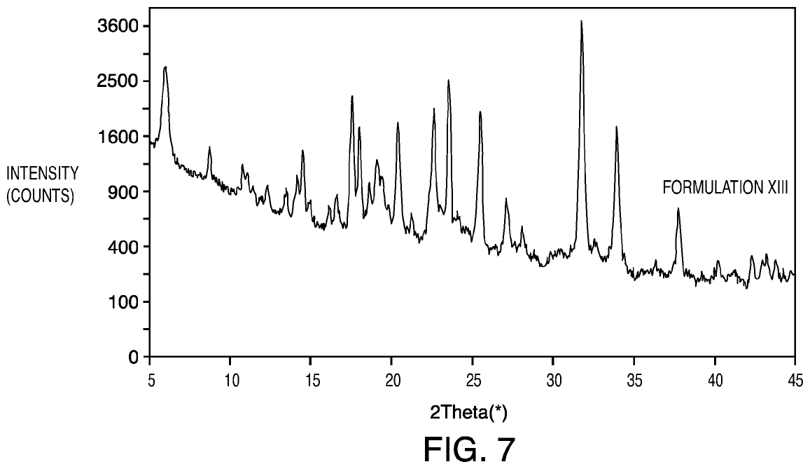

The crystallinity of Formulation XIII was assessed via XRD. The diffraction pattern of itraconazole is observed in the formulation, suggesting the milling or spray drying process does not affect the solid-state of itraconazole. Additional peaks observed in the pattern correspond to the additional excipients in the formulations. (FIG. 7)

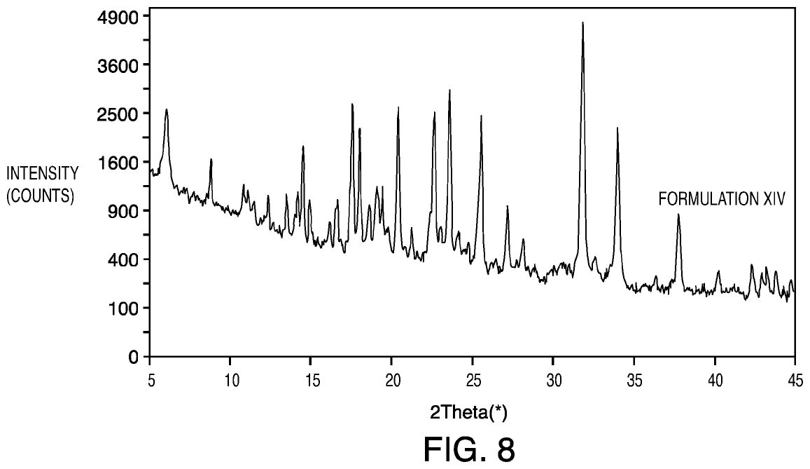

The crystallinity of Formulation XIV was assessed via XRD. The diffraction pattern of itraconazole is observed in the formulation, suggesting the milling or spray drying process does not affect the solid-state of itraconazole. Additional peaks observed in the pattern correspond to the additional excipients in the formulations. (FIG. 8)

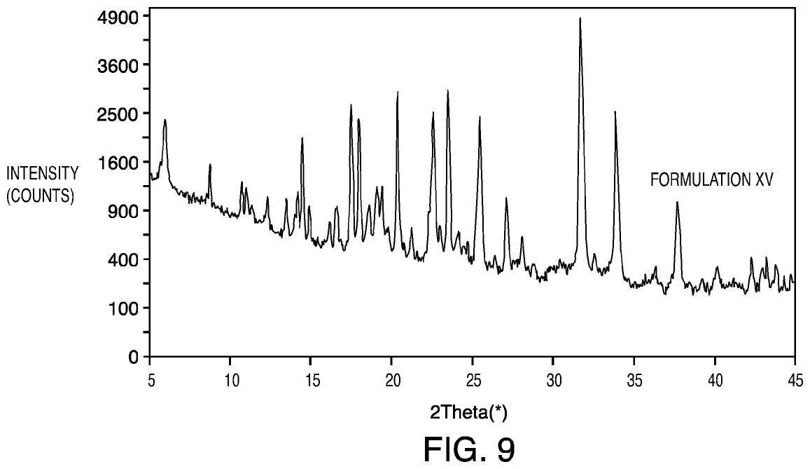

The crystallinity of Formulation XV was assessed via XRD. The diffraction pattern of itraconazole is observed in the formulation, suggesting the milling or spray drying process does not affect the solid-state of itraconazole. Additional peaks observed in the pattern correspond to the additional excipients in the formulations. (FIG. 9)

### Example 8. Dry Powder Formulation of Polysorbate 80-Stabilized Crystalline Itraconazole Comprising Sodium Sulfate/Leucine and Reduced Levels of Polysorbate 80

### A. Powder Preparation

The microcrystalline itraconazole for Formulation XVI was prepared using a Qualification Micronizer jet mill (Sturtevant, Hanover, MA USA). The feed pressure was set to 90 psig and the grind pressure was set to 40 psig. Itraconazole (SMS Pharma, Lot ITZ-0715005) was continuously fed into the mill until about 60 g of itraconazole was milled. The final median particle size (Dv(50)) of the milled API was about 1510 nm.

The microcrystalline itraconazole for Formulation XVI was then compounded into a suspension consisting of 10 wt % itraconazole and 0.25 wt % polysorbate 80 in deionized water. The batch size was 440 g. The polysorbate 80 was dissolved in 89.75% DI water via magnetic stir bar, then the micronized itraconazole was slowly added and allowed to mix until the suspension was observed to be visually dispersed and homogeneous.

A feedstock solution was prepared and used to manufacture a dry powder composed of nanocrystalline itraconazole, polysorbate 80 and other additional excipients. A drug load of 50 wt % itraconazole, on a dry basis, was targeted. The feedstock solution that was used to spray dry particles were made as follows. The required quantity of water was weighed into a suitably sized glass vessel. The excipients were added to the water and the solution allowed to stir until visually clear. The itraconazole-containing suspension was then added to the excipient solution and stirred until visually homogenous. The feedstock was then spray-dried. The feedstock volume was 3000 g, which supported a manufacturing campaign of approximately one hour. Table 34 lists the components of the feedstock used in preparation of the dry powder.

TABLE 34
Feedstock composition

| | | | | Sodium | | |
|---|---|---|---|---|---|---|
| | Water | Itraconazole | Polysorbate | sulfate | Leucine | Total mass |
| Formulation | (g) | (g) | 80 (g) | (g) | (g) | (gm) |
| XVI | 2964.2310 | 18.0225 | 0.4500 | 12.6 | 5.1 | 3000.3 |

A dry powder of Formulation XVI was manufactured from this feedstock by spray drying on the Niro Mobile Minor spray dryer (GEA Process Engineering Inc., Columbia, MD) with bag filter collection. The system was run in open-loop (single pass) mode using nitrogen as the drying and atomization gas. Atomization of the liquid feed utilized a Schlick 940-0 atomizer with a 1.0 mm liquid insert. The aspirator of the system was adjusted to maintain the system pressure at −2.0″ water column.

The following spray drying conditions were followed to manufacture the dry powder. For Formulations XVI, the liquid feedstock solids concentration was 1.2%, the process gas inlet temperature was 181° C. to 185° C., the process gas outlet temperature was 65° C., the drying gas flowrate was 80 kg/hr, the atomization gas flowrate was 250 g/min, the atomization gas backpressure at the atomizer inlet was 30.4 psig to 31.4 psig and the liquid feedstock flowrate was 50 mL/min. The resulting dry powder is reported in Table 35.

TABLE 35
Dry powder composition, dry basis

| Formulation | Dry Powder Composition (w/w), dry basis |
|---|---|
| XVI | 50% itraconazole, 35% sodium sulfate, |
| | 13.75% leucine, 1.25% polysorbate 80 |

### B. Powder Characterization

The bulk particle size characteristics for the formulation are found in Table 36. The span at 1 bar of 1.93 for Formulation XVI, indicates a relatively narrow size distribution. The 1 bar/4 bar dispersibility ratio of 1.03 for Formulation XVI, indicates the particle size is relatively independent of dispersion energy, a desirable characteristic which allows similar dispersion across a range of dispersion energies.

TABLE 36
Bulk particle size

| | 0.5 bar | | 1 bar | | 4 bar | | |
|---|---|---|---|---|---|---|---|
| | Dv[50] | | Dv[50] | | Dv[50] | | 1 bar:4 bar |
| Formulation | (μm) | Span | (μm) | Span | (μm) | Span | Dv[50] ratio |
| XVI | 2.13 | 1.77 | 2.02 | 1.93 | 1.96 | 1.87 | 1.03 |

The weight loss of Formulation XVI was measured via TGA and was found to be 0.37%.

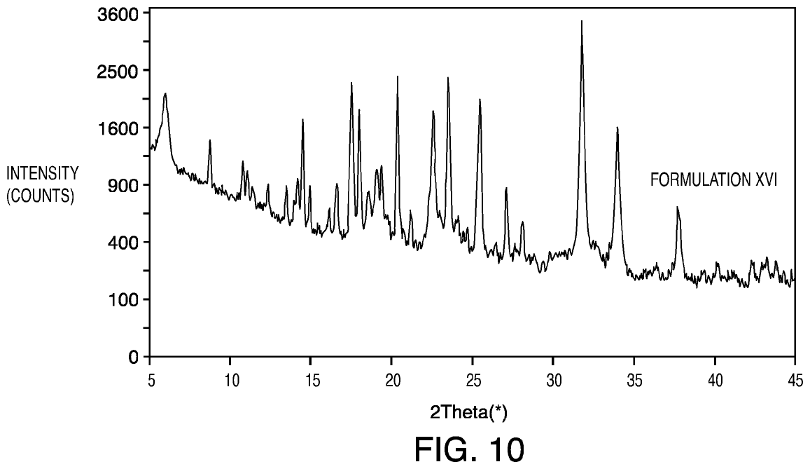

The crystallinity of Formulation XVI was assessed via XRD. The diffraction pattern of itraconazole is observed in the formulation, suggesting the milling or spray drying process does not affect the solid-state of itraconazole. Additional peaks observed in the pattern correspond to the additional excipients in the formulations. (FIG. 10)

### Example 9. Spray-Dried Dry Powder Formulation of Itraconazole, Sodium Sulfate and Leucine

### A. Powder Preparation

A feedstock solution utilizing a water-tetrahydrofuran (THF) co-solvent system was prepared and used to manufacture a dry powder composed of itraconazole, sodium sulfate and leucine. A drug load of 50 wt % itraconazole, on a dry basis, was targeted. The feedstock solution that was used to spray dry particles was made as follows. The required quantity of water was weighed into a suitably sized glass vessel. The excipients were added to the water and the solution allowed to stir until visually clear. The required amount of THE was weighed into a suitably sized glass vessel. The itraconazole was added to the THF and the solution allowed to stir until visually clear. The itraconazole-containing THE solution was then added to the excipient solution and stirred until visually homogenous. The feedstock was then spray-dried. The feedstock volume was 5 L, which supported a manufacturing campaign of approximately 8.5 hours. Table 37 lists the components of the feedstock used in preparation of the dry powder.

TABLE 37
Feedstock composition

| | | | | Sodium | | |
|---|---|---|---|---|---|---|
| | Water | Tetrahydrofuran | Itraconazole | sulfate | Leucine | Total |
| Formulation | (g) | (g) | (g) | (g) | (g) | mass (gm) |
| XIX | 2246.1 | 2444.3 | 30.1 | 21.0 | 9.0 | 4750.5 |

A dry powder of Formulation XIX was manufactured from this feedstock by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Buchi nozzle with 1.5 mm cap and 0.7 mm liquid tip. The aspirator of the system was adjusted to maintain the system pressure at −2.0″ water column.

The following spray drying conditions were followed to manufacture the dry powder. For Formulations XIX, the liquid feedstock solids concentration was 12.0 g/L, the process gas inlet temperature was 92° C. to 103° C., the process gas outlet temperature was 40° C., the drying gas flowrate was 17.0 kg/hr, the atomization gas flowrate was 2830 g/min, and the liquid feedstock flowrate was 10.0 mL/min. The resulting dry powder is reported in Table 38.

TABLE 38
Dry powder composition, dry basis

| Formulation | Dry Powder Composition (w/w), dry basis |
|---|---|
| XIX | 50% itraconazole, 35% sodium sulfate, 15% leucine |

### B. Powder Characterization

The bulk particle size characteristics for the formulation are found in Table 39. The span at 1 bar of 2.32 for Formulations XIX, indicates a relatively narrow size distribution. The 1 bar/4 bar dispersibility ratio of 1.12 for Formulations XIX, indicates the particle size is relatively independent of dispersion energy, a desirable characteristic which allows similar dispersion across a range of dispersion energies.

TABLE 39
Bulk particle size

| | 0.5 bar | | 1 bar | | 4 bar | | |
|---|---|---|---|---|---|---|---|
| | Dv[50] | | Dv[50] | | Dv[50] | | 1 bar:4 bar |
| Formulation | (μm) | Span | (μm) | Span | (μm) | Span | Dv[50] ratio |
| XIX | 2.67 | 2.28 | 2.39 | 2.32 | 2.14 | 2.25 | 1.12 |

The geometric particle size and capsule emitted powder mass (CEPM) measured and/or calculated at 60 liters per minute (LPM) and 30 LPM simulated patient flow rates were measured for the formulation and reported in Table 40. The small changes in CEPM and geometric size from 60 LPM to 20 LPM indicates that the dry powder is relatively independent of patient inspiratory flowrate, indicating that patients breathing in at varying flow rates would receive a relatively similar therapeutic dose.

TABLE 40
Emitted particle size

| | |
|---|---|
| 30 LPM | 60 LPM |

| | CEPM | Dv[50] | CEPM | Dv[50] |
|---|---|---|---|---|
| Formulation | (%) | (μm) | (%) | (μm) |
| XIX | 98.1 | 3.94 | 99.2 | 3.21 |

The aerodynamic particle size, fine particle fractions and fine particle doses measured and/or calculated with a Next Generation Impactor (NGI) are reported in Table 41. The fine particle dose for Formulation XIX indicates a high percentage of the nominal dose, which is filled into the capsule reaches the impactor stages (41.1%), and so would be predicted to be delivered to the lungs. The MMAD of Formulation XIX was 3.80 microns, indicating deposition in the central and conducting airways.

TABLE 41
Aerodynamic particle size

| | MMAD | FPD <5 μm |
|---|---|---|
| Formulation | (μm) | (% nominal dose) |
| XIX | 3.80 | 41.1 |

The weight loss of Formulation XIX was measured via TGA and was found to be 0.37%.

The itraconazole content of Formulation XIX was measured with HPLC-UV and is 99.0%.

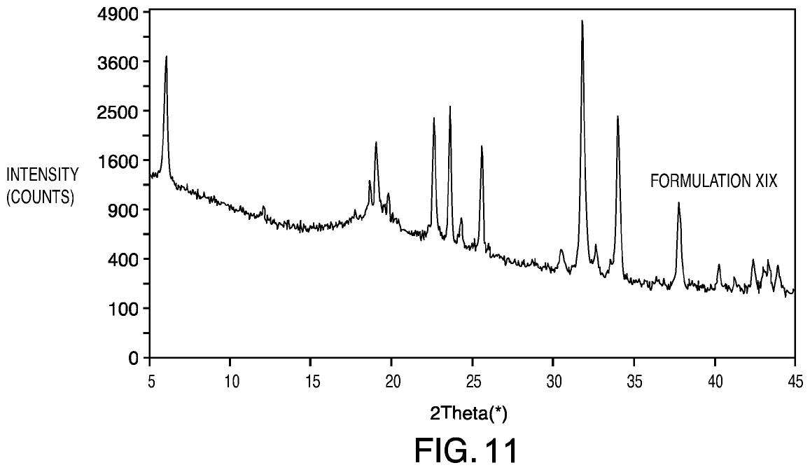

The crystallinity of Formulation XIX was assessed via XRD (FIG. 11). No itraconazole peaks are observed, indicating no appreciable levels of itraconazole are present in the formulation. As shown, all peaks observed in the formulation correspond to the excipients. The solid state of the itraconazole in Formulation XIX can therefore be characterized as amorphous.

### Example 10. In Vitro Dissolution Study of Dry Powder Formulations Comprising Itraconazole

### A. In Vitro Dissolution Study

An in vitro model was utilized to provide a predictive test to understand the dissolution of itraconazole. Drug dissolution is a prerequisite for cellular uptake and/or absorption via the lungs. Hence, the dissolution kinetics of itraconazole plays a key role in determining the extent of its absorption from the respiratory tract. For dry particles containing itraconazole that are delivered to the respiratory tract as an aerosol, the fate of the itraconazole in those particles is dependent on their physicochemical properties. For the itraconazole in the aerosolized dry particles to exert a local effect in the lung, the dry particle must first undergo dissolution for the itraconazole to be present in the lung fluid and tissue. However, once dissolution of the itraconazole into the lung fluid has occurred, the itraconazole may further become available for permeation and systemic absorption. The rate of dissolution of itraconazole was predicted to be proportional to its solubility, concentration in surrounding liquid film and area of solid-liquid interface. Solubility is dependent on compound, formulation and physical form of the drug. The total liquid volume in the lung is 10-30 mL with a lining fluid volume corresponding to ca. 5 μL/cm², which may compromise the solubilization and subsequent absorption of poorly soluble molecules such as itraconazole.

The following in vitro dissolution model was used to understand the dissolution properties of itraconazole containing dry powder aerosols. The aerosol particles were collected at well-defined aerosol particle size distribution (APSD) cut-offs using the Next Generation Impactor (NGI) (Copley Scientific, UK), and then the dissolution behavior simulated using model lung fluid.

A UniDose™ (Nanopharm, Newport, United Kingdom) aerosol dose collection system combined with a modified next generation impactor (NGI) was used to uniformly deposit the impactor stage mass (ISM), which is defined as the dose collected on and below stage 2 of a next generation impactor, onto a suitable filter for subsequent dissolution studies in a USP V—Paddle over disk (POD) apparatus.

### B. Materials and Methods for the In Vitro Dissolution Study

The materials used in the study are shown in Table 42. The powder formulations, capsules and packaging materials were equilibrated at 22.5±2.5° C. and 30±5% RH. Formulations were encapsulated into size 3 HPMC capsules under the same conditions. The fill weight for the powder preparations was 10 mg. The formulations were aerosolized from capsules in a unit-dose, capsule-based DPI device (RS01, Plastiape, Osnago, Italy).

One capsule of each formulation was aerosolized at 60 L/min (4 L inhaled volume) using the Plastiape RS01 dry powder inhaler (DPI). The aerosol dose was collected in the UniDose system. One milliliter of the suspension formulations was aerosolized into the cNGI at 15 L/min using a Micro Mist™ Nebuliser (Hudson RCI, Temecula, CA, USA). The UniDose collection system was used to uniformly deposit the whole impactor stage mass (i.e., below stage 2 of an NGI) onto a glass microfiber filter membrane, which can be seen as where the circles (representing particles or droplets) deposit. The filter was placed into a disk cassette and dissolution studies were undertaken using 500 ml PBS pH 7.4+2.0% SDS in a USP Apparatus II POD (Paddle Over Disk, USP V) at 37° C. For all studies, sink conditions were maintained within the vessel. Samples were taken at specified time points and tested for drug content on an Agilent (Santa Clara, CA, USA) 1260 Infinity series HPLC. Data has been presented as raw cumulative mass and cumulative mass percentage (%) at 240 minutes (mins).

TABLE 42
Formulations tested.

| Formulation | Description of Itraconazole |
|---|---|
| XIX | Dry powder formulation (amorphous itraconazole) |
| XI | Nano formulation with Oleic Acid (Wet milling |
| | process #1) - Dry Powder Formulation |
| XII | Nano formulation with Polysorbate 80 (Wet milling |
| | process #1) - Dry Powder Formulation |
| X | Liquid Nanosuspension Formulation |
| XIII | Nano formulation with Polysorbate 80 (Microfluidics |
| | process #1) - Dry Powder Formulation |
| XIV | Nano formulation with Polysorbate 80 (Wet milling |
| | process #2) - Dry Powder Formulation |
| XV | Micro formulation with polysorbate 80 (Jet milling |
| | process #1) - Dry Powder Formulation |
| IX | Liquid Microsuspension Formulation |
| Pure ITZ | 100% API of itraconazole as-received from manufacturer |

### C. Results of the UniDose POD Dissolution Studies of the Impactor Stage Mass (ISM) of the Formulations

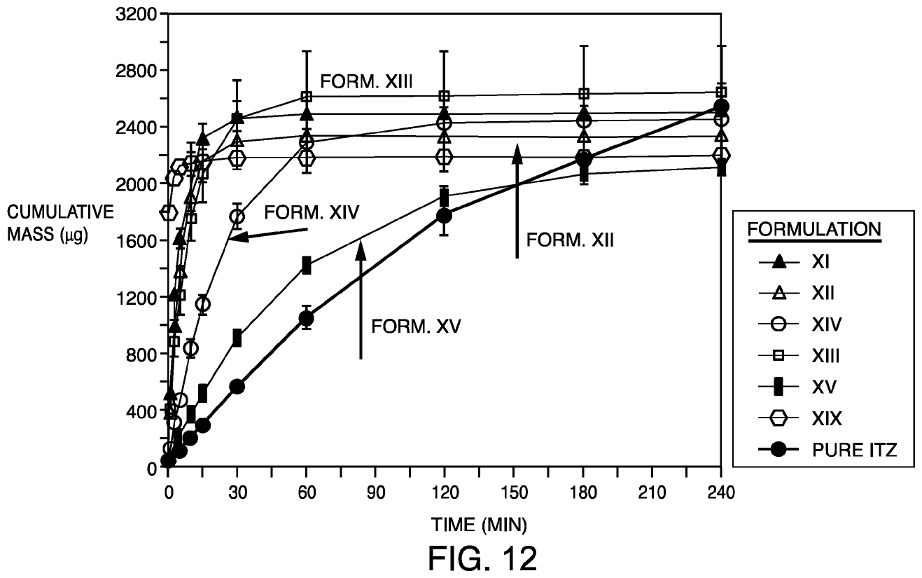

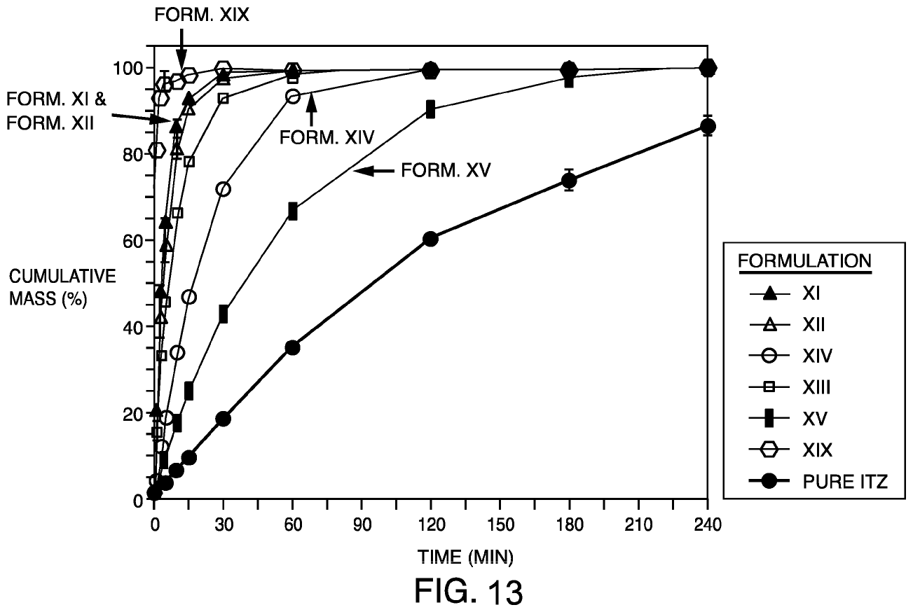

The raw cumulative mass and percentage cumulative mass dissolution plots of the ISM of formulations are shown in FIGS. 12 and 13, respectively. The UniDose ISM and dissolution half-life of each dry powder is summarized in Table 42. Particle size of the itraconazole crystal in suspension and the specific surface area (SSA) of the itraconazole crystals estimated using the measured particle size distributions are also shown in Table 43.

Based on the cumulative mass data, the collected ISM of the formulations ranged between 2.1-2.6 mg itraconazole. These data suggested that the aerosolization efficiency of the formulations was approximately 50% based on the nominal dose, since the itraconazole loading in each particle was 50% and the nominal dose was 5 mg of itraconazole (10 mg of powder).

The rate of dissolution of Formulation XIX was the fastest and more than 80% of the drug had dissolved within the first time-point. Due to the rapid dissolution kinetics of formulation XIX it was not possible to calculate the dissolution half-life. The dissolution half-life of the other dry powders showed the following rank order in their dissolution kinetics:

$$XI > XII > XIII > XIV > XV > Pure ITZ$$

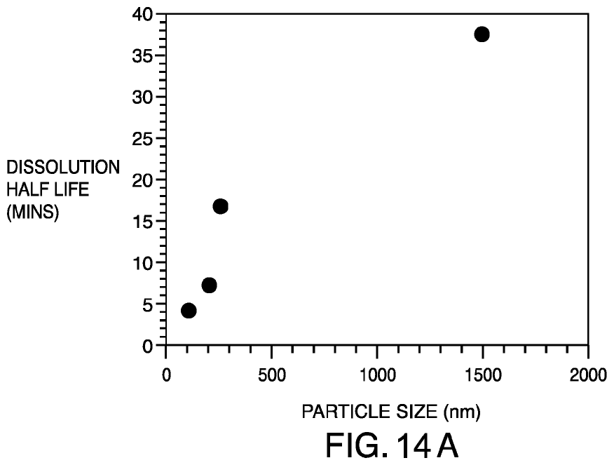

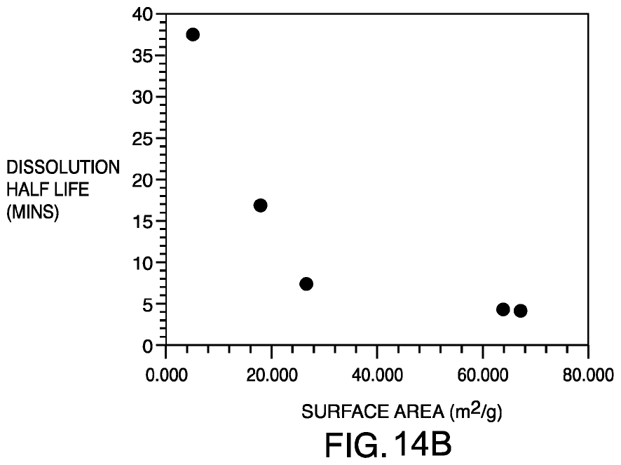

The data shown in FIGS. 12 and 13 was also evaluated for the relationship between the particle size of formulations XI, XII, XIII, XIV, and XV and their respective dissolution half-life, as shown in FIG. 14A. These data suggest a good correlation between the particle size of the itraconazole crystal and the dissolution half-life. FIG. 14B shows the relationship between specific surface area of the itraconazole crystals and dissolution half-life. These data suggest that as the surface area of the particles in the formulation increases that dissolution half-life shortens. These data highlight that that the particle size and thus surface area of the drug substance affects the dissolution behavior of the formulation.

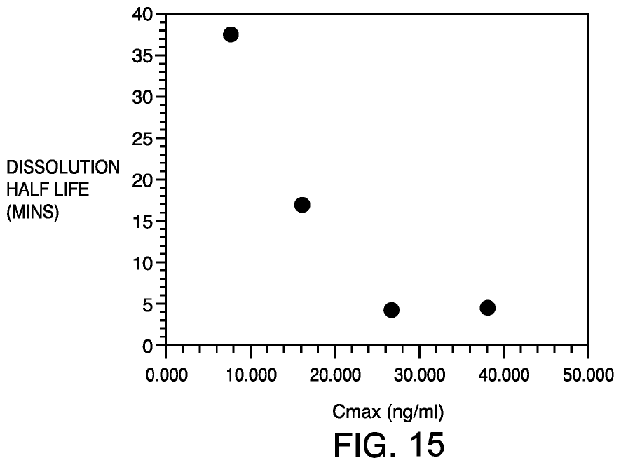

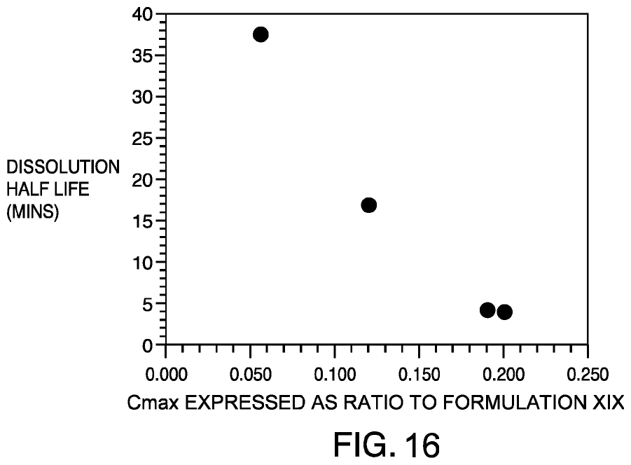

Based on the pharmacokinetic data shown in Example 14, Formulation XIX had the highest systemic exposure. This correlated with the dissolution data, which suggested that this formulation had rapid dissolution kinetics. The relationship between the dissolution half-life of the other dry powders and Cₘₐₓ or Cₘₐₓ expressed as a ratio of the Cₘₐₓ response of Formulation XIX are shown in FIGS. 15 and 16, respectively. There was an inverse relationship between the dissolution half-life and Cₘₐₓ, which suggested that a faster rate of dissolution resulted in higher systemic exposure. The correlation between the Cₘₐₓ ratio to the systemic response of Formulation XIX with dissolution half-life was stronger. These data suggest that the systemic exposure responses of itraconazole formulations are modulated by their dissolution behavior and in turn the physicochemical properties of the formulations.

TABLE 43
Particle size, UniDose ISM and dissolution
half-life of each formulation listed below.

| | PSD of | SSA of | | |
|---|---|---|---|---|
| | Itraconazole | Itraconazole | UniDose | Dissolution |
| | Crystal | Particle | ISM | Half-Life |
| Formulations | (nm) | (m²/g) | (mg) | (mins) |
| XIV | 258 | 18.0 | 2.45 | 16.84 |
| XI | 126 | 67.1 | 2.51 | 4.13 |
| XII | 132 | 64.1 | 2.35 | 4.35 |
| XIII | 198 | 26.6 | 2.65 | 7.39 |
| Pure ITZ | Not Known | Not Known | 2.55 | 101.79 |
| XV | 1600 | 3.86 | 2.12 | 37.53 |
| XIX | Not Applicable | Not Applicable | 2.20 | NA |

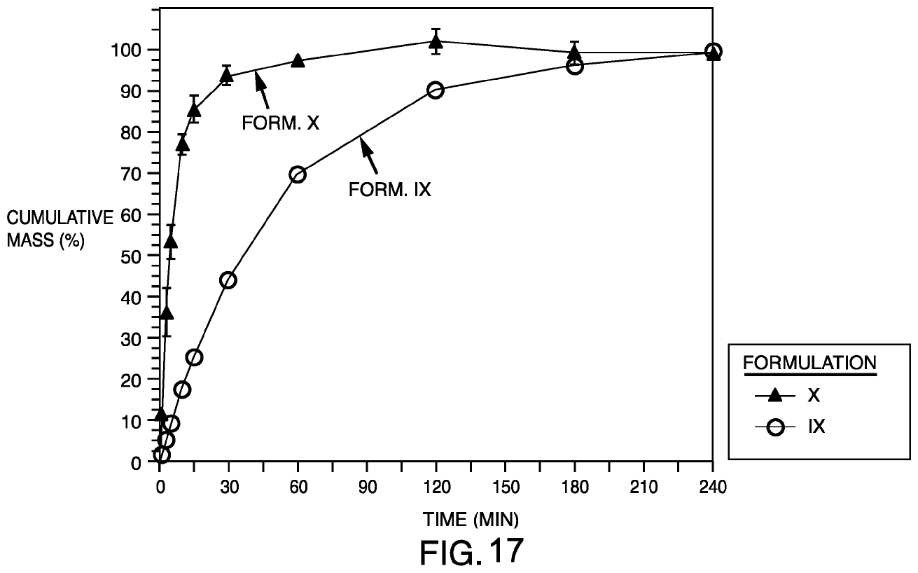

The raw cumulative mass percentage dissolution plots of the ISM of Nano-suspension Formulation and the Micro-suspension Formulation determined by UniDose POD is shown in FIG. 17.

The rate of dissolution of Nano-suspension Formulation was faster than the Micro-suspension Formulation. The dissolution half-life of the Nano-suspension Formulation and the Micro-suspension Formulation were 5.3 and 35.5 mins, respectively.

### Example 11. In Vitro Dissolution and Permeability Study of Dry Powder Formulations Comprising Crystalline Itraconazole

### A. In Vitro Dissolution and Permeability Study

A bio-relevant dissolution testing system was used based on mimicking the air-liquid interface at the respiratory epithelium interface using a cell-based in vitro method. A modified next generation impactor that incorporated cell culture plates onto collection stages (cNGI) was used to uniformly deposit materials onto the cell cultures. Dissolution and permeation of the drug through the epithelial cell monolayer was measured.

### B. Materials and Methods for the In Vitro Dissolution and Permeability Study

Epithelial cell monolayers grown at the air-liquid interface in Snapwell™ (Corning Costar, Massachusetts, USA) permeable insert were integrated into the cNGI. Calu-3 cell line (ATCC, LGC Standards, Teddington, UK) (passage 32-50) were grown in minimum essential medium (MEM) supplemented with non-essential amino acids, 10% (v/v) fetal bovine serum, 1% (v/v) penicillin-streptomycin and 1% (v/v) Fungizone antimycotic and maintained in a humidified atmosphere of 95%/5% Air/CO₂, respectively, at 37° C. Cells were seeded on to Snapwell inserts at a density of 5×10⁵ cells.cm⁻² and cultured under air-interfaced conditions from day 2 in culture for 12 days. The transepithelial electrical resistance (TEER) was measured using an EVOM2 chopstick electrode connected to an EVOM2 Epithelial Voltohmmeter (World Precision Instruments, Hitchin, United Kingdom) and monolayers with a TEER above 450 Ω·cm² were deemed confluent.

Snapwells containing Calu-3 ALI cells were transferred to a modified NGI cup and placed into stage 4 of the NGI (Copley Scientific, Nottingham, UK). A single capsule of the dry powder was aerosolized into the cNGI at 60 L/min for 4 seconds. One milliliter of the suspension formulations was aerosolized into the cNGI at 15 L/min using a Micro Mist Nebulizer (Hudson RCI, Temecula, CA, USA).

The materials used in the study are shown in Table 41. The dry powders, capsules and packaging materials were equilibrated at 22.5±2.5° C. and 30±5% RH. Formulations were encapsulated into size 3 HPMC capsules under the same conditions. The fill weight for the powder preparations was 10 mg. The formulations were aerosolized from capsules in a unit-dose, capsule-based DPI device (RS01™, Plastiape, Osnago, Italy). One capsule of each formulation was aerosolized at 60 L/min (4 L inhaled volume) using the Plastiape RS01™ dry powder inhaler (DPI).

Post-dosing of the dose on to the Snapwells from stage 4, the Snapwells were transferred to 6-well plates, which contained 2 mL of PBS pH 7.4+2.0% SDS maintained at 37° C. Basolateral samples were taken at different time points and drug content was measured on an Agilent (Santa Clara, CA, USA) 1260 Infinity series HPLC. Total dose delivered to the cells was measured from the total amount of drug dissolved over the time-course and from lysing cells post experimentation.

### C. Results of the cNGI Integrated Dissolution and Permeability Studies of Powder Formulations of Itraconazole

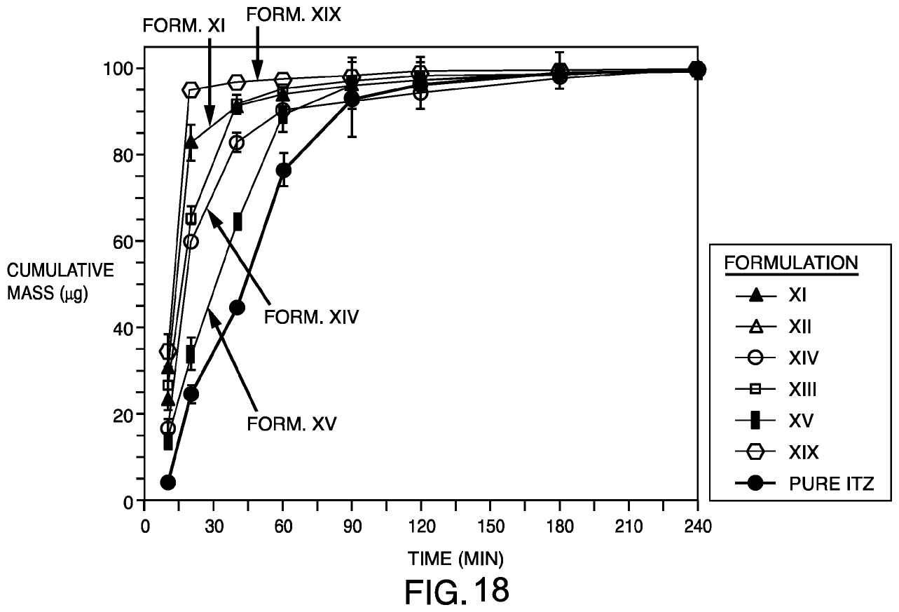

The cumulative mass percent (%) of the total recovered dose plots of the dry powders comprising itraconazole delivered to the cells on stage 4 are shown in FIG. 18. These data suggested differences between the dissolution and permeability kinetics of the different formulations. The as-received pure itraconazole had slower dissolution and permeability kinetics than the other formulations, whilst Formulation XIX had the fastest dissolution and permeability kinetics.

To understand the cNGI data for the different formulations, we utilised the data to calculate the rate of diffusion of the drug substance by taking into consideration loaded dose differences. This was done using the following equation:

$$Rate of Diffusion = \frac{J}{{AC}_{0}}$$

where J is the flux (gradient of the cNGI dissolution/permeability profile), A is the area of the barrier and C₀ is the loaded dose. These data are summarised in Table 44, which shows that the rate of diffusion for the formulations followed the rank order:

$$XIX > XI > XII > XIII > XIV > XV > Pure ITZ$$

TABLE 44
Particle size and rate of diffusion of exemplary dry powders

| | PSD of Itraconazole | Rate of |
|---|---|---|
| Formulations | Crystal (nm) | Diffusion (cm/s) |
| XI | 126 | 6.11 |
| XII | 132 | 4.88 |
| XIII | 198 | 4.82 |
| Pure ITZ | Not Known | 1.81 |
| XV | 1600 | 2.52 |
| XIX | Not Known | 7.01 |

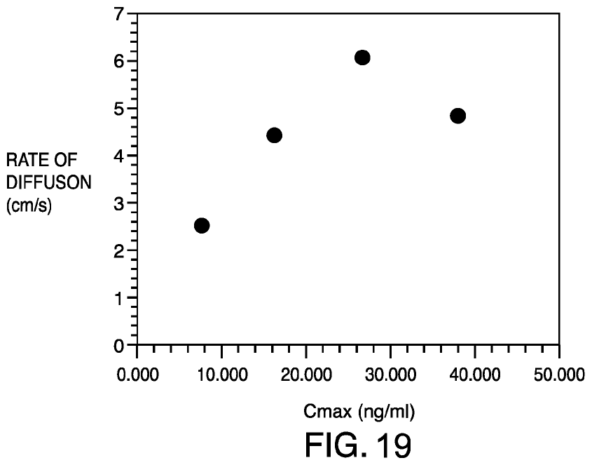

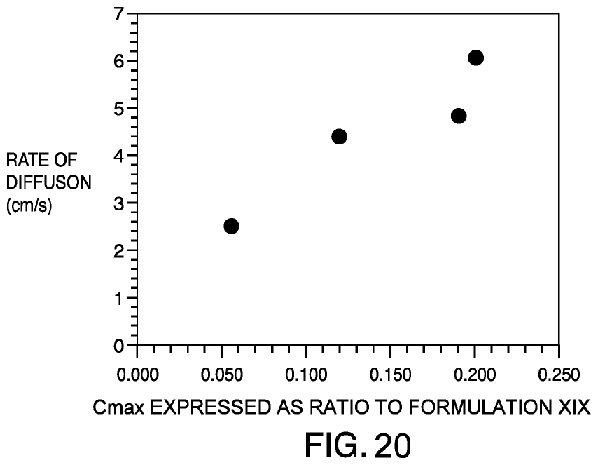

Based on the pharmacokinetic data shown in Example 14, Formulation XIX had the highest systemic exposure. This correlated with the rate of diffusion of this formulation, which suggested that this formulation had rapid dissolution and permeation kinetics. The relationship between the rate of diffusion of the other dry powders and Cₘₐₓ or Cₘₐₓ expressed as a ratio of the Cₘₐₓ response of Formulation XIX are shown in FIGS. 19 and 20, respectively. There was a relationship between the rate of diffusion and Cₘₐₓ, which suggested that a faster rate of diffusion resulted in higher systemic exposure. The correlation between the Cₘₐₓ ratio to the systemic response of Formulation XIX with the rate of diffusion was stronger.

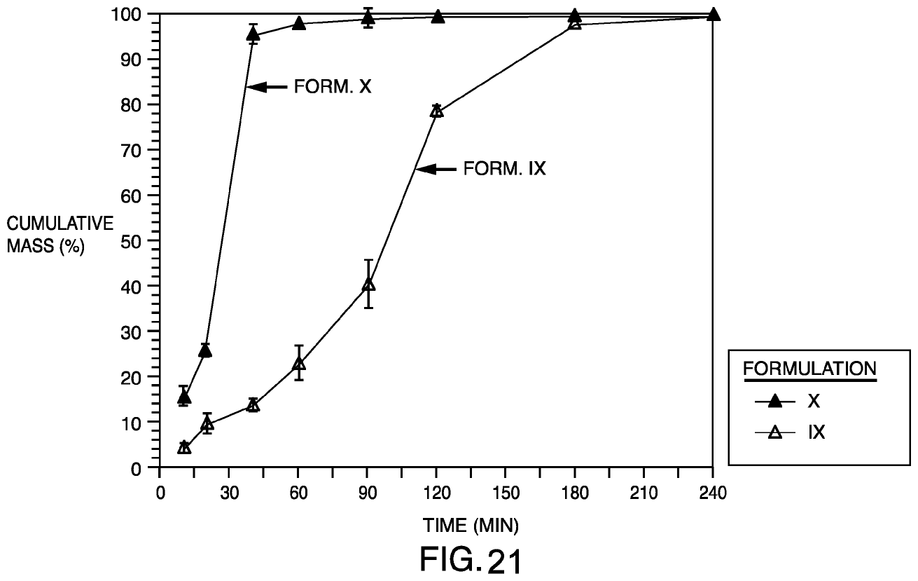

The raw cumulative mass percentage dissolution plots of the ISM of Nano-suspension Formulation and the Micron-suspension Formulation determined by cNGI is shown in FIG. 21. The cNGI data suggests that the rate of diffusion of the Nano-suspension Formulation was faster than the Micro-suspension Formulation.

### Example 12. Single Dose Inhalation PK Study in Rats

### A. Materials and Methods

Blood and lung tissue samples were taken from rats following a single inhalation administration of each of five different itraconazole formulations over a 60-minute exposure period in order to assess the systemic exposure of male rats to itraconazole and its metabolite, hydroxy-itraconazole, at a nominal dose level of 5 mg/kg. Plasma concentrations of itraconazole and hydroxy-itraconazole in samples taken at the end of the exposure period, and up to 96 hours after the end of exposure were measured by validated LC-MS/MS methods.

### B. Results—Plasma

Maximum mean plasma concentrations (Cₘₐₓ) of itraconazole and the areas under the mean plasma concentration-time curves estimated up to the time of the last quantifiable sample (AUCₗₐₛₜ) are summarized in Table 45.

TABLE 45
Plasma Cₘₐₓ and AUCₗₐₛₜ

| | Itraconazole | | Hydroxy-itraconazole | |
|---|---|---|---|---|
| | Cₘₐₓ | AUCₗₐₛₜ | Cₘₐₓ | AUCₗₐₛₜ |
| Formulation | (ng/mL) | (ng · h/mL) | (ng/mL) | (ng · h/mL) |
| XIX | 123 | 1170 | 231 | 4950 |
| XII | 38.1 | 921 | 95.1 | 3550 |
| XI | 26.7 | 529 | 101 | 2770 |
| XIV | 16.2 | 353 | 56.2 | 1630 |
| XV | 7.65 | 157 | 38.5 | 1390 |

The ratios of the maximum mean plasma concentrations (Cₘₐₓ) and areas under the mean plasma concentration-time curves (AUCₗₐₛₜ) in each group relative to the Cₘₐₓ and AUCₗₐₛₜ values for the group receiving Formulation XIX, based on Cₘₐₓ and AUCₗₐₛₜ values corrected for the differences in the doses received, are presented in Table 46.

TABLE 46
Plasma Cₘₐₓ and AUCₗₐₛₜ, both relative to Formulation XIX.

| | Itraconazole | | Hydroxy-itraconazole | |
|---|---|---|---|---|
| | Cₘₐₓ | AUCₗₐₛₜ | Cₘₐₓ | AUCₗₐₛₜ |
| Formulation | (ng/mL) | (ng · h/mL) | (ng/mL) | (ng · h/mL) |
| XIX | 1 | 1 | 1 | 1 |
| XII | 0.19 | 0.49 | 0.25 | 0.44 |
| XI | 0.20 | 0.42 | 0.41 | 0.52 |
| XIV | 0.12 | 0.28 | 0.23 | 0.31 |
| XV | 0.056 | 0.12 | 0.15 | 0.25 |

The rate (Cₘₐₓ) and extent (AUCₗₐₛₜ) of systemic exposure of rats to itraconazole were highest following exposure to Formulation XIX. Cₘₐₓ and AUCₗₐₛₜ were similar following exposure to Formulation XII and Formulation XI and were slightly lower following exposure to Formulation XIV. Cₘₐₓ and AUCₗₐₛₜ were lowest following exposure to Formulation XV. A similar pattern was observed for the rate and extent of systemic exposure to hydroxy-itraconazole, although Cₘₐₓ and AUCₗₐₛₜ values following exposure to Formulation XII were lower than those following exposure to Formulation XI and slightly higher than following exposure to Formulation XIV.

### C. Results—Lung Tissue

Maximum mean lung tissue concentrations (Cₘₐₓ) of itraconazole and the areas under the mean lung tissue concentration-time curves estimated up to the time of the last quantifiable sample (AUCₗₐₛₜ) are summarized in Table 47.

TABLE 47
Lung Tissue Cₘₐₓ and AUCₗₐₛₜ

| | |
|---|---|
| Itraconazole | Hydroxy-itraconazole |

| | Cₘₐₓ | AUCₗₐₛₜ | Cₘₐₓ | AUCₗₐₛₜ |
|---|---|---|---|---|
| Formulation | (ng/g) | (ng · h/g) | (ng/g) | (ng · h/g) |
| XIX | 23600 | 86900 | 733 | 13400 |
| XII | 91300 | 2400000 | 260 | 10100 |
| XI | 66800 | 1090000 | 285 | 6720 |
| XIV | 53500 | 1810000 | 144 | 3970 |
| XV | 36400 | 1600000 | 114 | 3310 |

The ratios of the maximum mean lung tissue concentrations (Cₘₐₓ) and areas under the mean lung tissue concentration-time curves (AUCₗₐₛₜ) in each group relative to the Cₘₐₓ and AUCₗₐₛₜ values for the group receiving Formulation XIX, based on Cₘₐₓ and AUCₗₐₛₜ values corrected for the differences in the doses received, are presented in Table 48.

TABLE 48
Lung Tissue Cₘₐₓ and AUCₗₐₛₜ, both relative to Formulation XIX.

| | Itraconazole | | Hydroxy-itraconazole | |
|---|---|---|---|---|
| Formulation | Cₘₐₓ ratio | AUCₗₐₛₜ ratio | Cₘₐₓ ratio | AUCₗₐₛₜ ratio |
| XIX | 1 | 1 | 1 | 1 |
| XII | 2.4 | 17.0 | 0.22 | 0.46 |
| XI | 2.6 | 11.7 | 0.36 | 0.47 |
| XIV | 2.6 | 19.4 | 0.36 | 0.28 |
| XV | 1.4 | 16.5 | 0.14 | 0.22 |

The rate (Cₘₐₓ) and extent (AUCₗₐₛₜ) of local exposure of the lungs of rats to itraconazole were lowest following exposure to Formulation XIX. Cₘₐₓ and AUCₗₐₛₜ were generally similar following exposure to Formulation XII, Formulation XI and Formulation XIV, although AUCₗₐₛₜ following exposure to Formulation XII was somewhat lower than that following exposure to the other two formulations. Following exposure to Formulation XV, Cₘₐₓ was only slightly higher than that following exposure to Formulation XIX and was lower than the values for the other formulations, while AUCₗₐₛₜ was higher than that following exposure to Formulation XIX and was broadly similar to that following exposure to the other formulations. The Cₘₐₓ and AUCₗₐₛₜ values for hydroxy-itraconazole were highest following exposure to Formulation XIX, and were lower following exposure to Formulation XII, Formulation XI, Formulation XIV and Formulation XV, but were broadly similar for all four of these formulations.

The ratios of the AUCₗₐₛₜ values in lung to those the corresponding values in plasma are presented in Table 49.

TABLE 49
Ratios of AUCₗₐₛₜ for lung tissue to plasma tissue

Lung tissue:plasma ratio

| Formulation | Itraconazole | Hydroxy-itraconazole |
|---|---|---|
| XIX | 74 | 2.7 |
| XII | 2600 | 2.8 |
| XI | 2100 | 2.4 |
| XIV | 5100 | 2.4 |
| XV | 10000 | 2.4 |

The lung tissue; plasma ratios for itraconazole were lowest following exposure to Formulation XIX, were similar following exposure to Formulation XII and Formulation XI and were somewhat higher following exposure to Formulation XIV. The highest ratio was observed following exposure to Formulation XV. The lung tissue; plasma ratios for hydroxy-itraconazole were similar following exposure to each formulation and were much lower than the ratios observed for itraconazole.

### Conclusions

The systemic exposure of rats to itraconzole was highest following administration of Formulation XIX. Systemic exposure was similar following inhalation administration of Formulation XII and Formulation XI and was slightly lower following administration of Formulation XIV. Systemic exposure was lowest following administration of Formulation XV. A similar pattern was observed for systemic exposure to hydroxy-itraconazole, systemic exposure following administration of Formulation XII was lower than that following administration of Formulation XI and slightly higher than that following administration of Formulation XIV.

The local exposure of the lungs of rats to itraconzole was lowest following exposure to Formulation XIX. Local exposure was generally similar following administration of Formulation XII, Formulation XI and Formulation XIV. Following administration of Formulation XV, the maximum concentrations were only slightly higher than those following administration of Formulation XIX and were lower than the values for the other formulations, while AUCₗₐₛₜ values were higher than that following exposure to Formulation XIX and were broadly similar to those following exposure to the other formulations. Local exposure to hydroxy-itraconazole was highest following administration of Formulation XIX, and was lower following administration of Formulation XII, Formulation XI, Formulation XIV and Formulation XV, but was broadly similar for all four of these formulations.

### Example 13. Dry Powder Formulations of Amorphous Itraconazole Prepared for Use in 28-Day Toxicity Studies

### A. Powder Preparation

A feedstock solution utilizing a water-tetrahydrofuran (THF) co-solvent system was prepared and used to manufacture a dry powder composed of itraconazole, sodium sulfate and leucine. A drug load of 50 wt % itraconazole, on a dry basis, was targeted. The feedstock solution that was used to spray dry particles was made as follows. The required quantity of water was weighed into a suitably sized glass vessel. The excipients were added to the water and the solution allowed to stir until visually clear. The required amount of THE was weighed into a suitably sized glass vessel. The itraconazole was added to the THF and the solution allowed to stir until visually clear. The itraconazole-containing THE solution was then added to the excipient solution and stirred until visually homogenous. The feedstock was then spray-dried. The individual feedstock volume was 9.5625 L. Fourteen of these feedstocks were prepared for a total of 133.875 L which supported a manufacturing campaign of approximately 30 hours. Table 50 lists the components of each feedstock used in preparation of the dry powder.

TABLE 50
Feedstock composition

| | | | | Sodium | | |
|---|---|---|---|---|---|---|
| | Water | Tetrahydrofuran | Itraconazole | sulfate | Leucine | Total |
| Formulation | (g) | (g) | (g) | (g) | (g) | mass (gm) |
| XX | 4295.379 | 4676.636 | 57.375 | 40.163 | 17.213 | 9086.766 |

A dry powder of Formulation XX was manufactured from this feedstock by spray drying on the Niro Mobile Minor spray dryer (GEA Process Engineering Inc., Columbia, MD) with bag filter collection. The system was run in open-loop (single pass) mode using nitrogen as the drying and atomization gas. Atomization of the liquid feed utilized a Niro atomizer with a 1.0 mm liquid insert. The aspirator of the system was adjusted to maintain the system pressure at −2.0″ water column.

The following spray drying conditions were followed to manufacture the dry powder. For Formulation XX, the liquid feedstock solids concentration was 12 g/L, the process gas inlet temperature was 120° C. to 140° C., the process gas outlet temperature was 40° C., the drying gas flowrate was 80 kg/hr, the atomization gas flowrate was 352.2 g/min, the atomization gas backpressure at the atomizer inlet was 45 psig to 57 psig and the liquid feedstock flowrate was 75 mL/min. The resulting dry powder is reported in Table 51. The itraconazole in the formulation was amorphous.

TABLE 51
Dry powder composition, dry basis

| Formulation | Dry Powder Composition (w/w), dry basis |
|---|---|
| XX | 50% itraconazole, 35% sodium sulfate, 15% leucine |

### B. Powder Characterization

The bulk particle size characteristics for the formulation are found in Table 52. The span at 1 bar of 1.83 for Formulation XX, indicates a relatively narrow size distribution. The 1 bar/4 bar dispersibility ratio of 1.06 for Formulation XX, indicates the particle size is relatively independent of dispersion energy, a desirable characteristic which allows similar dispersion across a range of dispersion energies.

TABLE 52
Bulk particle size

| | 0.5 bar | | 1 bar | | 4 bar | | |
|---|---|---|---|---|---|---|---|
| | Dv[50] | | Dv[50] | | Dv[50] | | 1 bar:4 bar |
| Formulation | (μm) | Span | (μm) | Span | (μm) | Span | Dv[50] ratio |
| XX | 1.84 | 1.83 | 1.58 | 1.81 | 1.50 | 1.82 | 1.06 |

The weight loss of Formulation XX was measured via TGA and was found to be 0.34%.

The itraconazole content of Formulation XX was measured with HPLC-UV and is 100.9% of nominal.

### Example 14. Dry Powder Formulations of Crystalline Itraconazole Prepared for Use in 28-Day Toxicity Studies

### A. Powder Preparation

The nanocrystalline itraconazole for Formulation XXI was prepared as a suspension comprising 25 wt % itraconazole (SMS Pharma lot ITZ-0715005) and 2.5 wt % polysorbate. The polysorbate 80 was dissolved in 72.5% deionized water via magnetic stir bar, then the itraconazole was added and suspensded by stirring with a magenetic stir bar. Once all of the itraconazole was suspended, the formulation was processed on the Netzsch MiniCer using 0.2 mm grinding media (TOSOH, Tokyo, Japan) with 90% chamber fill. The following conditions were used to manufacture the itraconazole suspension. The mill speed was 3000 RPM, the inlet pump speed was 100 RPM, the recirculating chiller was 10° C., the inlet air pressure was 4.5 bar, and run time was 30-40 minutes. Eight suspensions were processed this way and combined to make the final suspension lot. The final median particle size (Dv(50)) of the milled suspension was 130 nm.

The nanocrystalline itraconazole for Formulation XXII was prepared as a suspension comprising 10 wt % itraconazole and 0.7 wt % oleic acid, 1.5% ammonium hydroxide in deionized water. The oleic acid was dissolved in 87.8 deionized water via magnetic stir bar and then the ammonium hydroxide was added and dissolved via magnetic stir bar. Finally, the itraconazole was added and mixed with a magnetic stir bar to form a suspension. Once all of the itraconazole was suspended, the formulation was processed on the Netzsch MiniCer using 0.5 mm grinding media (TOSOH. Tokyo, Japan) with 90% chamber fill. The following conditions were used to manufacture the itraconazole suspension The mill speed was 3000 RPM, the inlet pump speed was 100 RPM, the recirculating chiller was 10° C. the inlet air pressure was 4.5 bar, and run time was 200-240 minutes. Eight suspensions were processed this way and combined to make the final suspension lot. The final median particle size (Dv(50)) of the milled suspension was 115 nm.

The microcrystalline itraconazole for Formulation XXIII was prepared using a Qualification Micronizer jet mill (Sturtevant, Hanover, MA USA). The feed pressure was set to 85 psig and the grind pressure was set to 45 psig. Itraconazole was continuously fed into the mill until 480.0 g of itraconazole was milled. The final median particle size (Dv(50)) of the milled API was 1640 nm. The micronized itraconazole for Formulation XXIII was then compounded into a suspension consisting of 10 wt % itraconazole and 0.25 wt % polysorbate 80 in deionized water. The batch size was 4800 g. The polysorbate 80 was dissolved in 88.75% deionized water via magnetic stir bar, then the itraconazole was slowly added and allowed to mix until the suspension was observed to be visually dispersed and homogeneous.

Feedstock suspensions were prepared and used to manufacture dry powders composed of crystalline itraconazole, and other additional excipients. A drug load of 50 wt % itraconazole, on a dry basis, was targeted. The feedstock suspensions that were used to spray dry particles were made as follows. The required quantity of water was weighed into a suitably sized glass vessel. The excipients were added to the water and the solution was allowed to stir until visually clear. The itraconazole-containing suspension was then added to the excipient solution and stirred until visually homogenous. The feedstocks were then spray-dried. Feedstocks were stirred while spray dried. The individual feedstock masses for Formulation XXI were 7.5 kg each. Six of these feedstocks were spray dried, which supported a manufacturing campaign of fifteen hours. The individual feedstock masses for Formulation XXII were 6.0 kg each. Three of these feedstocks were spray dried, which supported a manufacturing campaign of six hours. The individual feedstock masses for Formulation XXIII were 8.0 kg each. Four of these feedstocks were spray dried, which supported a manufactured campaign of approximately 11 hours. Table 53 and 54 list the components of the feedstocks used in preparation of the dry powders.

TABLE 53
Feedstock compositions for formulations comprising polysorbate 80

| | | | | Sodium | | |
|---|---|---|---|---|---|---|
| | Water | Itraconazole | Polysorbate | sulfate | Leucine | Total mass |
| Formulation | (g) | (g) | 80 (g) | (g) | (g) | (gm) |
| XXI | 7275.0 | 112.5 | 11.25 | 78.75 | 22.5 | 7500.0 |
| XXIII | 7904.0 | 48.0 | 1.2 | 33.6 | 13.2 | 8000.0 |

TABLE 54
Feedstock compositions for formulations comprising oleic acid

| | | | Oleic | Ammonium | Sodium | | Total |
|---|---|---|---|---|---|---|---|
| | Water | Itraconazole | acid | hydroxide | sulfate | Leucine | mass |
| Formulation | (g) | (g) | (g) | (g) | (g) | (g) | (gm) |
| XXII | 5806.74 | 90.00 | 6.18 | 13.38 | 63.00 | 20.82 | 6000.12 |

Dry powders of Formulations XXI-XXIII were manufactured from these feedstocks by spray drying on the Niro Mobile Minor spray dryer (GEA Process Engineering Inc., Columbia, MD) with bag filter collection. The system was run in open-loop (single pass) mode using nitrogen as the drying and atomization gas. Atomization of the liquid feed utilized a Niro two fluid nozzle atomizer with a 1.0 mm liquid insert. The aspirator of the system was adjusted to maintain the system pressure at −2.0″ water column.

The following spray drying conditions were followed to manufacture the dry powders. For Formulations XXI and XXII, the liquid feedstock solids concentration was 3%, the process gas inlet temperature was 170° C. to 190° C., the process gas outlet temperature was 65° C., the drying gas flowrate was 80.0 kg/hr, the atomization gas flowrate was 250.0 g/min, and the liquid feedstock flowrate was 50.0 g/min. For Formulation XXIII, the liquid feedstock solids concentration was 1.2%, the process gas inlet temperature was 170-190° C., the process gas outlet temperature was 65° C., the drying gas flowrate was 80.0 kg/hr, the atomization gas flowrate was 250.0 g/min, and the liquid feedstock flowrate was 50.0 g/min. The resulting dry powders are reported in Table55.

TABLE 55
Dry powder composition, dry basis

| Formu- | | Dry Powder Composition |
|---|---|---|
| lation | Description | (w/w), dry basis |
| XXI | Nanocrsytalline, | 50% Itraconazole, 35% sodium sulfate, |
| | PS 80 stabilizer | 15% leucine, 5.0% polysorbate 80 |
| XXII | Nanocrystalline, | 50% itraconazole, 35% sodium sulfate, |
| | oleic acid stabilizer | 11.57% leucine, 3.43% oleic acid |
| XXIII | Microcrystalline, | 50% itraconazole, 35% sodium sulfate, |
| | PS80 stabilizer | 13.75% leucine, 1.25% polysorbate 80 |

### B. Powder Characterization

The bulk particle size characteristics for the three formulations are found in Table 56. The span at 1 bar of less than 2.05 for Formulations XXI-XXIII indicates a relatively narrow size distribution. The 1 bar/4 bar dispersibility ratio less than 1.25 for Formulations XXI-XXIII indicate that they are relatively independent of dispersion energy, a desirable characteristic which allows similar particle dispersion across a range of dispersion energies.

TABLE 56
Bulk particle size

| | 0.5 bar | | 1 bar | | 4 bar | | |
|---|---|---|---|---|---|---|---|
| | Dv[50] | | Dv[50] | | Dv[50] | | 1 bar:4 bar |
| Formulation | (μm) | Span | (μm) | Span | (μm) | Span | Dv[50] ratio |
| XXI | 2.35 | 1.86 | 2.10 | 1.97 | 1.94 | 2.10 | 1.08 |
| XXII | 2.33 | 1.90 | 2.13 | 2.01 | 2.00 | 2.07 | 1.07 |
| XXIII | 2.15 | 1.82 | 2.03 | 1.88 | 1.89 | 1.88 | 1.08 |

The weight loss of Formulations XXII-XXIII were measured via TGA and are detailed in Table 57.

TABLE 57
Weight loss (%) via TGA

| Formulation | Weight loss via TGA (%) |
|---|---|
| XXI | 0.45 |
| XXII | 0.32 |
| XXIII | 0.37 |

The itraconazole content of Formulations XXI-XXIII were measured with HPLC-UV and are detailed in Table 58.

TABLE 58
Itraconazole content

| | Itraconazole content |
|---|---|
| Formulation | (% label claim) |
| XXI | 98.50 |
| XXII | 100.50 |
| XXIII | 101.20 |

### Example 17. 28-Day Inhalation Toxicity Studies A and B in Rats

### A. Materials and Methods

In order to assess both the plasma and lung pharmacokinetics as well as the potential for local tissue toxicity, two separate 28-day studies were performed. In the first study, 28-Day Study A, 5 groups of animals were dosed daily for 28 days with either air or placebo controls or one of three doses of Formulation XX. In the second study, 28-Day Study B, 7 groups of rats were dosed with one of three formulations of crystalline nanoparticulate itraconazole, daily for 28 days, or in the case of one group, every three days. Groups and achieved doses are detailed in Tables 59 and 60.

TABLE 59
Dose Groups in 28-Day Study A

| Group | | Achieved Total Delivered API Dose Level |
|---|---|---|
| No. | Formulation | (mg/kg) |
| 1 | Air Control | 0 |
| 2 | Placebo Control | 0 |
| 3 | XX | 5.8 |
| 4 | XX | 22 |
| 5 | XX | 49 |

TABLE 60
Dose Groups in 28-Day Study B

| Group | | Achieved Total Delivered API Dose Level |
|---|---|---|
| No. | Formulation | (mg/kg) |
| 1 | XXI | 5.2 |
| 2 | XXI | 14.8 |
| 3 | XXI | 38.3 |
| 4 | XXI × 3 days | 14.8 |
| 5 | XXIII | 5.0 |
| 6 | XXIII | 14.5 |
| 7 | XXII | 14.7 |

In both studies, blood and lung tissue samples were taken from rats following the first and last inhalation administration of each formulations in order to assess the lung and systemic exposure and accumulation of itraconazole in male and female rats. In addition, pulmonary tissue samples, including the larynx, trachea, tracheal bifurcation (carina) and lungs, were collected from all animals 24 hours after the last dose in order to assess microscopic pathology changes resulting from the dosing. Plasma and lung concentrations of itraconazole in samples were measured by validated LC-MS/MS methods.

### B. Results—Plasma

Maximum mean plasma concentrations (Cₘₐₓ) of itraconazole and the areas under the mean plasma concentration-time curves estimated up to the time of the last quantifiable sample (AUC₀₋ₗₐₛₜ) on Days 1 and 28 in male and female rats from 28-Day Study A, with amorphous itraconazole, are summarized in Table 61 and from 28-Day Study B, with crystalline itraconazole, are summarized in Table 62.

TABLE 61
Plasma Cₘₐₓ and AUC₀₋ₗₐₛₜ for Itraconazole

| | | Cₘₐₓ (ng/mL) | | | | AUC_{0-Tlast} (ng · h/mL) | | | |
|---|---|---|---|---|---|---|---|---|---|
| | Dose level | Day 1 | | Day 28 | | Day 1 | | Day 28 | |
| Formulation | (mg/kg/day) | Males | Females | Males | Females | Males | Females | Males | Females |
| XX | 5.8 | 188 | 283 | 117 | 430 | 1100 | 3840 | 914 | 6310 |
| XX | 22.0 | 348 | 620 | 335 | 1310 | 2710 | 11200 | 2110 | 22200 |
| XX | 49.0 | 510 | 928 | 602 | 2270 | 4990 | 17800 | 3800 | 35200 |

TABLE 62
Plasma Cₘₐₓ and AUC₀₋ₗₐₛₜ for Itraconazole

| | | Cₘₐₓ (ng/mL) | | | | AUC_{0-Tlast} (ng · h/mL) | | | |
|---|---|---|---|---|---|---|---|---|---|
| | Dose level | Day 1 | | Day 28 | | Day 1 | | Day 28 | |
| Formulation | (mg/kg/day) | Males | Females | Males | Females | Males | Females | Males | Females |
| XXI | 5.2 | 17.1 | 56.4 | 25.6 | 299 | 263 | 1080 | 376 | 5600 |
| XXI | 14.8 | 32.9 | 119 | 90.4 | 726 | 471 | 2310 | 1540 | 12700 |
| XXI | 38.3 | 110 | 292 | 261 | 1290 | 2020 | 4660 | 5260 | 23900 |
| XXI* | 14.8 | 49.0 | 36.3 | 317 | 339 | 785 | 718 | 2830 | 4920 |
| XXIII | 5.0 | 13.8 | 28.3 | 98.1 | 247 | 204 | 514 | 1020 | 5020 |
| XXIII | 14.5 | 39.6 | 134 | 110 | 550 | 473 | 2390 | 2140 | 11000 |
| XXII | 14.7 | 39.1 | 76.9 | 164 | 526 | 560 | 1330 | 2900 | 11700 |

*Dosed every three days

Despite differences in the absolute achieved doses between the various formulations, it is clear that the peak (Cₘₐₓ) and total (AUC₀₋ₗₐₛₜ) systemic exposure for Formulation XXI is higher than that for any of the crystalline formulations, both after a single dose (Day 1) and repeat dosing (Day 28). Table 63 below summarizes the dose-normalized average Cₘₐₓ and AUC₀₋ₗₐₛₜ for itraconazole for each of the formulations from both 28-day studies using the target dose of 15 mg/kg/day from each study. Normalization was achieved by dividing the exposures measured by the actual achieved dose for each study on each day.

TABLE 63
Dose-normalized plasma Cₘₐₓ and AUC₀₋ₗₐₛₜ, for each of the crystalline formulations.

| | Day 1 | | | | Day 28 | | | |
|---|---|---|---|---|---|---|---|---|
| | Cₘₐₓ (ng/mL/) | | AUC₀₋ₗₐₛₜ (ng · h/mL) | | Cₘₐₓ (ng/mL) | | AUC₀₋ₗₐₛₜ (ng · h/mL) | |
| Formulation | M | F | M | F | M | F | M | F |
| XX | 17.40 | 28.18 | 135.50 | 509.09 | 18.61 | 68.95 | 117.22 | 1168.42 |
| XXI | 2.51 | 8.62 | 35.95 | 167.39 | 6.19 | 46.54 | 105.48 | 814.10 |
| XXI × 3 days | 3.77 | 2.65 | 60.38 | 52.41 | 20.58 | 20.42 | 183.77 | 296.39 |
| XXIII | 1.96 | 5.08 | 28.15 | 134.27 | 7.68 | 43.47 | 138.06 | 658.68 |
| XXII | 2.72 | 5.03 | 38.89 | 86.93 | 10.51 | 31.69 | 185.90 | 704.82 |

The dose normalized Cₘₐₓ and AUC₀₋ₗₐₛₜ for itraconazole systemic exposure in rats on Day 1 were highest following exposure to Formulation XX. By Day 28, Formulation XX still generally showed higher dose normalized Cₘₐₓ and AUC₀₋ₗₐₛₜ relative to the crystalline formulation XXI, particularly in females, though the difference was less pronounced with a slightly higher value for AUC₀₋ₗₐₛₜ in males for some formulations. These data demonstrate that, for a given achieved delivered lung dose, the systemic exposure that results from inhalation of crystalline formulations is generally less than that for Formulation XX. However, given that the systemic exposure is dependent upon both dissolution rate of the material in the lung and the permeability of the lung tissue, it also demonstrates that, over time, the crystalline formulations do show adequate dissolution and permeation of the tissue to narrow or abolish the difference providing confidence that the crystalline formulations are not simply insoluble deposits in the lung.

### C. Results—Lung Tissue

The Days 1 and 28 trough mean lung tissue concentrations (23 hours after the end of the previous dose) in each group expressed as ratio to the corresponding mean plasma concentration values at the same time point are presented in Table 64.

TABLE 64
Lung:plasma concentration ratio for each formulation.

| | |
|---|---|
| Day 1 Trough | Day 28 Trough |

| Formulation | M | F | M | F |
|---|---|---|---|---|
| XX | 15.9 | 3.7 | 421.6 | 13.8 |
| XXI | 1400 | 818 | 3349 | 1348 |
| XXI × 3 d | 897 | 611 | 3783 | 596 |
| XXIII | 15262 | 1500 | 10651 | 3926 |
| XXII | 3543 | 248 | 1201 | 155 |

The lung tissue; plasma ratios for itraconazole were lowest following exposure to Formulation XX and were consistently much higher for all of the crystalline formulations on both Days 1 and 28. These data indicate that the crystalline formulations provide substantially higher lung exposure with less systemic exposure at the doses tested, increasing the exposure at the site of action while minimizing the potential for unwanted effects of systemic exposure.

### D. Results—Lung Pathology

In 28-Day study A, Formulation XX-related microscopic findings were present in respiratory tissues at ≥5 mg/kg/day. Minimal to slight granulomatous inflammation was present at all doses and macrophages and multinucleated giant cells frequently contained intracytoplasmic spicules. At the highest dose, where a 28-day recovery period was included, these only partially recovered. The pathology recorded was considered adverse at all doses due to its dispersed presentation and the fact that it did not fully resolve during the recovery period. The spicular formations noted in the pathology would appear to be itraconazole that, we theorize, are formed when the amorphous material supersaturates the lung lining fluid and interstitial space leading to crystallization of the API after multiple doses. Shorter duration exposure studies with the same formulation showed no such findings.

In 28-Day Study B, Formulations XXI and XXIII were associated with minimal adverse accumulations of foamy macrophages in the lungs only at 40 mg/kg/day with Formulation XXI, the only formulation dosed at that level. There was no clear difference in the incidence and severity of findings between rats dosed with Formulations XXI-XXIII at comparable dose levels. Overall, the No Observed Adverse Effect Level (NOAEL) was approximately 15 mg/kg/day for all three of the crystalline formulations tested.
Pathological findings related to the amorphous compositions in the respiratory tract of rats had a different character from those induced by crystalline formulations, with findings in the latter group more related to a clearance response to accumulated material in the lumen of the airway versus granulomatous inflammation within the mucosa. In addition, amorphous formulation-related findings involved more regions in the respiratory tract and were adverse at a lower dose.

### Conclusions

The systemic exposure, i.e., plasma levels of rats to itraconazole was highest following administration of Formulation XX. Systemic exposure was generally less following inhalation administration of Formulations XXI-XXIII, though by Day 28 of dosing the differences were less than after a single dose. Lung exposure, however, was markedly and consistently higher with Formulations XXI-XXIII relative to Formulation XX. When comparing lung and systemic exposure, the ratio for Formulation XX favored lung exposure over systemic. However, the lung; plasma ratio was substantially greater for each of the crystalline formulations, XXI-XXIII. These data indicate that the crystalline formulations provide substantially higher local concentrations of itraconazole, while resulting in the same or less systemic exposure as Formulation XX.

The amorphous nature of the itraconazole in Formulation XX leads to increased solubility and rapid transit through the lung to the systemic circulation as evidenced by the significantly higher systemic exposure on Day 1. Formulation XX dosing also resulted in local toxicity in the form of spicular deposits in the mucosa leading to granulomatous inflammation that was adverse at all doses tested, and as low as 5 mg/kg/day. With the use of crystalline nanoparticles in Formulations XXI-XXIII, the lung retention was substantially greater, leading to higher local exposure than the amorphous formulations with generally the same or less systemic exposure. This change in exposure profile has the advantage of increasing efficacy in the lung with the unwanted effects of systemic itraconazole exposure no worse and possibly minimized further relative to Formulation XX. In addition, Formulations XXI-XXIII showed much lower potential for adverse microscopic pathology findings, despite the substantially higher local exposure.

### Summary In-Vitro and In-Vivo Example Summary

The investigation of the effects of the physical form of itraconazole within the dry powder involved an iterative progression through in-vitro dissolution and permeability studies and in-vivo single and multiple dose pharmacokinetic and toxicity studies. The in-vitro dissolution studies demonstrated that the physical form of itraconazole, as well as the size of crystalline particles within the formulation, play an important role in determining the rate of dissolution as well as the rate at which the delivered material would be expected to pass through the lung and into the systemic circulation. These data demonstrate the ability to control key aspects of both the lung and systemic exposure to allow the modulation of both efficacy as well as potentially the modulation of adverse findings. These in-vitro findings were tested in an in-vivo, single dose inhalation PK study, confirming that, when delivered via inhalation, the powders with crystalline itraconazole nanoparticles resulted in longer lung retention, leading to a higher lung to plasma ratio, as well as reduced peak and total systemic exposure relative to a formulation containing amorphous itraconazole after a single dose. The example summarizing the 28-day inhalation toxicity studies further demonstrated that the different exposure kinetics with amorphous and crystalline itraconazole in the dry powders, in terms of lung and systemic exposure, are retained over multiple days of dosing. In addition, when examining the microscopic pathology effects of the amorphous and crystalline materials after multiple days of dosing, it is clear that differences exist in both the nature and severity of these findings, with the crystalline material showing fewer adverse findings and only at higher lung exposures.

### Example 18. Human Simulation; Oral Inhalation and Oral Solution Administration

Certain assumptions were made for this human simulation. Pulmonary systemic absorption rates estimated using a rat model were used as input in the human simulations. Pulmonary solubility values from the rat model were used as the starting point for human simulations. Particle size distribution using Alberta Idealized Throat (MMAD and GSD) data was used along with ICRP66 model in GastroPlus™ to estimate deposition fraction in humans. An actual dose incorporating approximately 56% deposited in lung and approximately 12.6% in throat was used; the remaining percentage of the drug was assumed to be retained in apparatus.

Single dose pharmacokinetic parameters for Formulation XII was simulated over fourteen days of repeated exposure. A dose proportional increase in both total lung and plasma concentration was predicted from 5 mg to 20 mg. A similar half-life was predicted between lung and plasma

TABLE 65
Single Dose PK Parameters

| | | | | | | |
|---|---|---|---|---|---|---|
| Dose | Tₘₐₓ | Cₘₐₓ | DNCₘₐₓ | AUCₜ | AUC_{inf} | |
| (mg) | (h) | (ng/ml) | (ng/mL/mg) | (h × ng/mL) | (h × ng/mL) | T_{1/2} (h) |

Plasma Exposure Parameters for Inhaled Dose

| | | | | | | |
|---|---|---|---|---|---|---|
| 5 | 80.7 | 0.275 | 0.0551 | 68.4 | 87.2 | 128 |
| 10 | 81.5 | 0.549 | 0.0549 | 136 | 174 | 128 |
| 20 | 81.9 | 1.09 | 0.0547 | 272 | 347 | 128 |

Total Lung Exposure Parameters for Inhaled Dose

| | | | | | | |
|---|---|---|---|---|---|---|
| 5 | 0.00 | 2020 | 403 | 199000 | 222000 | 104 |
| 10 | 0.00 | 4030 | 403 | 398000 | 444000 | 104 |
| 20 | 0.00 | 8070 | 403 | 796000 | 888000 | 104 |

AUC_{inf}: area under the concentration-time curve from the time of drug administration (time 0) extrapolated to infinity; AUCₜ: area under the concentration-time curve from the time of drug administration (time 0) to a specific time (336 hours); Cₘₐₓ: maximum observed drug concentration; DNCmax: dose normalized Cmax; t_{1/2}: half-life; Tₘₐₓ: time to maximum observed concentration.

Dose proportional increases in the plasma and lung are predicted after multiple doses. After seven days of dosing, the model predicted accumulation in lung and larger accumulation in the plasma. Based on human predictions, some accumulation of undissolved drug within the alveolar interstitial region with subsequent doses was anticipated. Plasma concentration after oral solution administration was higher than plasma concentrations at either 5- or 20-mg oral inhalation dose levels. However, total lung concentration was higher after oral inhalation administration. As such, total lung; plasma ratio was significantly higher for oral inhalation administration when compared to oral solution administration.

TABLE 66
Multiple Dose PK Parameters

| | Day 1 | | Day 7 | | | | |
|---|---|---|---|---|---|---|---|
| Dose | Cₘₐₓ | AUCₜ^{a} | Cₘₐₓ | AUCₜ | AUC₀₋₂₄ | | |
| (mg) | (ng/mL) | (h × ng/mL) | (ng/mL) | (h × ng/mL) | (h × ng/mL) | AR Cmax | AR AUC₀₋₂₄ |

Plasma Exposure Parameters

| | | | | | | | |
|---|---|---|---|---|---|---|---|
| 5-mg | 0.230 | 4.11 | 1.78 | 409 | 41.3 | 7.71 | 10.1 |
| Inhalation | | | | | | | |
| Dose | | | | | | | |
| 20-mg | 0.902 | 16.3 | 7.05 | 1630 | 164 | 7.82 | 10.1 |
| Inhalation | | | | | | | |
| Dose | | | | | | | |

Total Lung Exposure Parameters

| | | | | | | | |
|---|---|---|---|---|---|---|---|
| 5-mg | 2010 | 36900 | 7190 | 904000 | 151000 | 3.58 | 4.10 |
| Inhalation | | | | | | | |
| Dose | | | | | | | |
| 20-mg | 8030 | 148000 | 28800 | 3620000 | 605000 | 3.58 | 4.10 |
| Inhalation | | | | | | | |
| Dose | | | | | | | |

^{A}AUC₀₋ₜ is AUC₀₋₂₄ for single dose.
Abbreviations: AR: accumulation ratio; AUC₀₋₂₄: area under the plasma concentration-time curve from time 0 to 24 hours; AUC_{inf}: area under the plasma concentration-time curve from the time of drug administration (time 0) extrapolated to infinity; AUCₜ: area under the concentration-time curve from the time of drug administration (time 0) to a specific time (24 hours for single dose and 360 hours for multiple dose); Cₘₐₓ: maximum observed drug concentration.

Table 67 show modelled human clinical data with Formulation XII inhaled (oral inhalation) at 5 or 20 mg doses or oral SPORANOX® (oral solution) at 200 mg, after a single dose. The lung; plasma ratios compare the AUC data in the lung and the plasma over the 7 day period for each dose. The ratios are substantially higher with an inhaled dose than with an oral dose. Even though the oral dose may achieve lung levels that might result in therapeutic lung levels, it would require a greater total dose delivered, as well as greater systemic exposure. Without wishing to be bound by theory, it is believed that the same lung exposure using 0.2 mg inhaled would be achieved with 200 mg orally).

TABLE 67
Oral versus Inhaled Single dose (AUC over 7 days)

| | | | Lung | Plasma | |
|---|---|---|---|---|---|
| | Dose | Dosing | AUC₀₋₁₆₈ | AUC₀₋₁₆₈ | Lung:Plasma |
| Formulation | (mg) | interval | (ng × h/mL) | (ng × h/mL) | Ratio |
| Oral | 5 | QD | 151000 | 41.4 | 3650 |
| Inhalation | | | | | |
| Oral | 20 | QD | 605000 | 164 | 3690 |
| Inhalation | | | | | |
| Oral | 200 | QD | 6370 | 2200 | 2.90 |
| Solution | | | | | |

Table 68 shows exposure over a 24-hour period at ‘steady state’ on Day 21. Dosing daily via inhalation was compared with possible dosing every other day (EOD) via inhalation. The EOD dosing option appeared to be half the daily dose, so it may be possible to refine the exposure kinetics based on regimen. Even with EOD dosing, the exposure in the lung is significantly higher compared to that seen after 200 mg oral dose daily.

TABLE 68
Oral versus Inhaled Steady State

| | | Lung | Plasma | |
|---|---|---|---|---|
| | Dosing | AUC₀₋₂₄ | AUC₀₋₂₄ | Lung:Plasma |
| Formulation | Interval | (ng × h/mL) | (ng × h/mL) | Ratio |
| 20-mg Inhalation | QD | 810000 | 281 | 2880 |
| Dose | | | | |
| 20-mg Inhalation | EOD | 451000 | 145 | 3110 |
| Dose | | | | |
| Standard Oral | QD | 6590 | 2280 | 2.90 |
| Solution Dose | | | | |

Abbreviations: AUC₀₋₂₄: area under the plasma concentration-time curve from time 0 to 24 hours; EOD: every other day; QD: once daily.

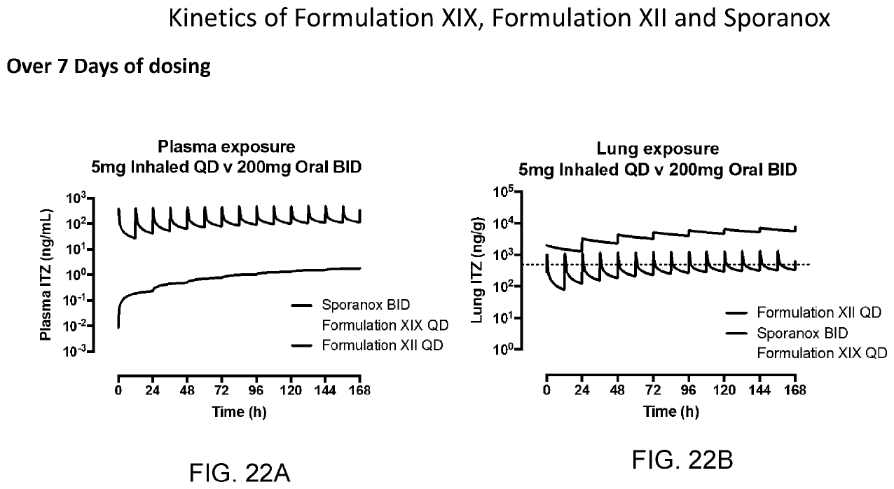

FIGS. 22A and 22B show the kinetics of three itraconazole-containing formulations at a 5 mg dose. On the left (FIG. 22A), the graph shows plasma exposure with the normal clinical SPORANOX® twice daily dosing regimen versus once daily Formulation XIX or Formulation XII dosing. Very clearly, the inhaled doses resulted in much lower systemic exposure and the Formulation XII formulation, though it ultimately does reach a similar trough exposure level as Formulation XIX, it does so with lower daily variability and much lower Cₘₐₓ.

On the right (FIG. 22B) is the lung exposure for the same doses and regimen—the dotted line approximates the Aspergillus MIC (˜500 ng/g or ng/ml). With oral dosing, the lung levels reach above the MIC, but for only short periods during the twice daily dosing and the majority of the exposure period the exposure is below the ‘efficacious’ level for treating a fungal infection. A very similar exposure profile in the lung was seen with 5 mg of Formulation XIX and SPORANOX®. However, the exposure profile, even at the lowest dose of 5 mg Formulation XII, resulted in lung exposure above the MIC and SPORANOX® for the entire 24 hour period, even on Day 1, and consistently across the 7 days of dosing. The antifungal efficacy of itraconazole is based on AUC/MIC, meaning the exposure above the MIC, in terms of both total exposure and time, are the critical factors determining efficacy. Clearly, the Formulation XII formulation achieves theoretical exposures much more conducive to antifungal efficacy than either the Formulation XIX formulation or oral SPORANOX® and all with greatly reduced systemic exposure. Without wishing to be bound by theory, it is believed these results demonstrate that dry powders disclosed herein containing angiogenesis inhibitor (e.g., itraconazole) can be used to achieve and/or maintain therapeutically effective concentrations of the angiogenesis inhibitor (e.g., itraconazole) in lung tissue, which is useful for treating a cancer, e.g., lung cancer such as NSCLC. In particular, studies of treating NSCLC in humans using high dose oral itraconazole have demonstrated a direct and significant correlation between reduction in tumor volume and tumor perfusion, decrease in the proangiogenic cytokines IL1b and GM-CSF, and reduction in tumor microvessel density (Gerber et al. Clin. Cancer Res. (2020) 26:6017-6027).

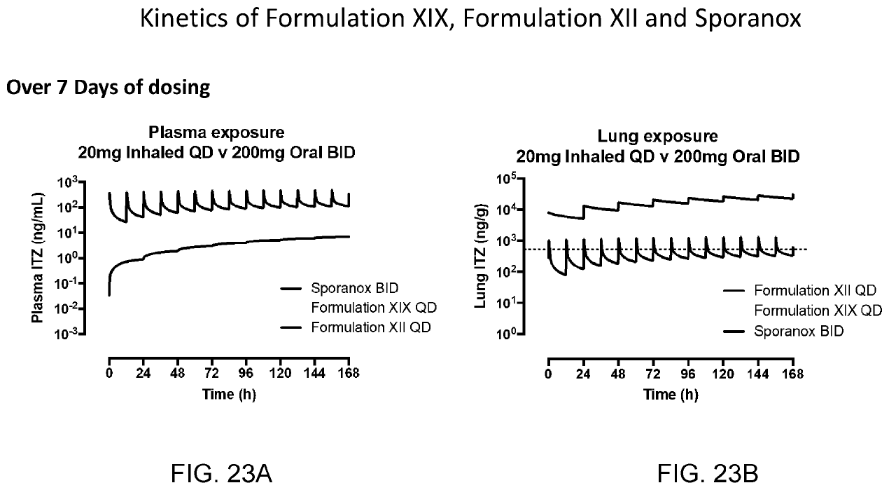

FIG. 23A and FIG. 23B show the kinetics of three itraconazole formulations at a 20 mg dose. The results and interpretation are similar to the 5 mg inhalation doses described above for FIG. 22A and FIG. 22B, except that a higher dose was administered and the corresponding lung and plasma exposure are increased for the inhaled doses. Using the higher dose, Formulation XIX achieves lung exposure above MIC over a 24 hour period and greater lung exposure than SPORANOX®. Conversely, the plasma exposure remains significantly below that of SPORANOX®. The 20 mg exposure of Formulation XII results in higher lung exposure than the 5 mg dose, that remains consistently above the MIC and the exposure of SPORANOX® throughout the time course.

### Example 19. Phase 1/1b; Safety-Tolerability Study

A safety, tolerability, and PK study in Healthy Volunteers and Asthmatics highlights the lung and plasma PK advantages over oral SPORANOX®. In part 1 of the study, a single ascending dose (5 mg, 10 mg, 25 mg, and 35 mg) of Formulation XII was administered to normal healthy volunteers (n=6/cohort). In part 2 of the study, multiple ascending doses (10) mg, 20 mg) of Formulation XII were administered to healthy volunteers (n=6/cohort), with an optional 3^{rd} cohort receiving up to 35 mg dose. The safety and tolerability of Formulation XII was assessed during the administration of Formulation XII up to 14 days at doses that were expected to provide more than five times higher lung exposure than oral SPORANOX®, and more than five times lower itraconazole plasma levels than observed with oral SPORANOX®.

Part 3 of the study assessed the safety and tolerability of Formulation XII or oral SPORANOX® administered as a single dose to asthmatics (n=16) in a cross-over design. Patients receiving 200 mg of oral SPORANOX® in the first period received a 20 mg dose of Formulation XII in the second period, while patients receiving 20 mg of Formulation XII in the first period received a 200 mg oral dose of SPORANOX® in the second period. Itraconazole levels in sputum and plasma were measured to assess lung and plasma exposure. This study confirmed that lung exposure for the Formulation XII resulted in lung concentrations that are greater than the minimum inhibitory concentration level (MIC) for A. fumigatus and higher than those achieved with oral SPORANOX®. The plasma exposure of itraconazole following administration of Formulation XII was more than 5× lower than that observed with oral SPORANOX® dosing. Without wishing to be bound by theory, it is believed these results also demonstrate that dry powders disclosed herein containing crystalline itraconazole can be used to achieve and maintain therapeutically effective concentrations of itraconazole in lung tissue to treat a lung cancer (e.g., NSCLC), whilst minimizing plasma concentrations and lowering risk of toxicity and side-effects.

### Example 20; Comparison of Respiratory Tract Findings from Two Rat and Three Dog Studies with Inhalation Exposures to Inhaled Itraconazole Formulations XIX and XII

Studies were conducted using inhaled dry powders comprising itraconazole, formulated using spray drying, in rats and dogs at two testing facilities. All studies included the same active pharmaceutical ingredient, but the formulation excipients in some cases and, in particular, the physiochemical properties of itraconazole in the particles varied. The studies and their results are summarized below.

### Rat Studies

### A 28-Day Inhalation Study with Formulation XIX in Rats Followed by a 28-Day Recovery Period

Rats were exposed to air, placebo, or itraconazole formulated as Formulation XIX at target doses of 5, 20, or 44 mg/kg/day, with itraconazole being 50% of the formulation concentration, for 28 days. Formulation XIX-related microscopic findings were present in the lungs and bronchi, larynx, and tracheal bifurcation at ≥5 mg/kg/day and in the trachea at ≥20 mg/kg/day. In the lungs and bronchi, minimal to slight granulomatous inflammation was present at itraconazole doses ≥5 mg/kg/day. The granulomatous inflammation was characterized by clusters of macrophages and multinucleated cells within the bronchiolar mucosa, often forming papillary outfoldings of the mucosa in the lumen. Macrophages and multinucleated giant cells frequently contained intracytoplasmic spicules. Alveolar macrophage aggregates were also present in the lungs at an incidence above background in rats dosed at ≥20 mg/kg/day. These macrophages were vacuolated, which gave the cytoplasm a foamy appearance.

In the larynx and tracheal bifurcation, minimal to slight granulomatous inflammation was present at itraconazole doses ≥5 mg/kg/day. As in the lung, this inflammation was characterized by clusters of macrophages and multinucleated giant cells with intracytoplasmic spicules within the mucosa. Similar minimal granulomatous inflammation was present in the tracheal mucosa of rats dosed at ≥20 mg/kg/day.

At the end of the 28-day recovery period, bronchiolar granulomatous inflammation was still present in rats dosed at 44 mg/kg/day; thus, the bronchiolar finding at this dose did not resolve during the recovery period. Granulomatous inflammation was not observed in the larynx, tracheal bifurcation, or trachea at the end of the recovery period, suggesting complete resolution in these tissues during the recovery period.

In summary, the main Formulation XIX-related finding was granulomatous inflammation characterized by mucosal macrophages and multinucleated giant cells with cytoplasmic spicules. This finding, which occurred in rats dosed at ≥5 mg/kg/day itraconazole was considered adverse at all doses because it occurred throughout the conducting airways from larynx to small bronchioles and did not resolve in the bronchioles during the recovery period in rats dosed at 44 mg/kg/day (other dose groups not examined at the end of the recovery period). Aggregates of alveolar macrophage with foamy cytoplasm were also present in the lungs at an incidence above background in rats dosed at ≥20 mg/kg/day at the terminal sacrifice.

### A 28-Day Inhalation Study with Formulation XII or Formulation XV in Rats

Rats were exposed to itraconazole formulated as Formulation XII at target doses of 5, 15, or 40 mg/kg/day or to Formulation XV at doses of 5 or 15 mg/kg/day for 28 days. In both cases, the itraconazole was 50% of the total formulation concentration. In addition, one group of rats was dosed at 15 mg/kg itraconazole as Formulation XII every three days. Formulation XII and Formulation XV-related minimal to mild accumulations of foamy macrophages were present in the lungs at 15 mg/kg/day with a higher incidence and severity in rats dosed with Formulation XII at 40 mg/kg/day. There may have been a minimal accumulation of foamy macrophages in the lung at 5 mg/kg/day Formulation XII or Formulation XV or 15 mg/k Formulation XII dosed every 3 days, but due to the lack of air or placebo control rats, it was not possible to determine if there was a test item-related effect at these doses. Mild subacute inflammation, which was considered test item related and adverse, was present in rats dosed at 40 mg/kg/day Formulation XII. It was not clear whether minimal subacute inflammation, which occurred in rats dosed at 15 mg/kg/day with Formulation XII or Formulation XV, was test item related. There was no clear difference in the incidence and severity of macrophage accumulation or subacute inflammation between male rats dosed with Formulation XII and Formulation XV at comparable dose levels. There was a suggestion of a higher severity and/or incidence of these findings in female rats dosed at 15 mg/kg/day with Formulation XV compared with Formulation XII.

### Dog Studies

### A 7-Day Inhalation Study of Formulation XIX in Dogs with a 14-Day Recovery Period

Dogs were exposed to itraconazole formulated as Formulation XIX at target doses of 5, 10, or 20 mg/kg/day for 7 days. The itraconazole formulation concentration was 50% of the total. A 14-day recovery group was included for dogs that were exposed at 5 mg/kg/day. Minimal to mild Formulation XIX-related acute inflammation, which was considered adverse, was present in both dogs (one male; one female) dosed at 20 mg/kg/day and minimal acute inflammation was present in the female dog dosed at 10 mg/kg/day. The acute inflammation was characterized by the presence of neutrophils, macrophages, and a few multinucleated giant cells that appeared to contain spicules in their cytoplasm (observed in a post-study slide review). Thus, the acute inflammation exhibited features of granulomatous inflammation. There were no test item-related findings in dogs dosed at 5 mg/kg/day at the terminal or recovery sacrifices.

### A 28-Day Inhalation Study of Formulation XIX in Dogs with a 28-Day Recovery Period

Dogs were exposed to air, placebo, or itraconazole formulated at target doses of 5, 10, or 20 mg/kg/day for 28 days. The itraconazole formulation concentration was 50% of the total. Minimal to mild, Formulation XIX-related, bronchiolar/peribronchiolar granulomatous inflammation was present in males and females at ≥5 mg/kg/day. Incidence and severity of this finding increased with dose in males at ≥5 mg/kg/day and females at 20 mg/kg/day. The granulomatous inflammation was within and surrounding terminal and respiratory bronchioles and was characterized by aggregates of macrophages and multinucleated giant cells with abundant, eosinophilic cytoplasm. Mild granulomatous inflammation, which occurred at ≥10 mg/kg/day was considered adverse. Granulomatous inflammation completely resolved during the recovery period.

### A 14-Day Inhalation Study of Formulation XII and Formulation XV in Dogs

Dogs were exposed to placebo or to itraconazole formulated as Formulation XII at target doses of 2, 6, or 20 mg/kg/day or Formulation XV at target doses of 6 or 20 mg/kg/day for 14 days. In addition, one group of dogs was dosed at a target dose of 6 mg/kg itraconazole as Formulation XII every three days. The itraconazole formulation concentration was 50% of the total in all cases. Test item-related respiratory tract findings were present in dogs administered 20 mg/kg/day Formulation XII or Formulation XV. Test item-related, mild, intra-alveolar, mixed cell inflammation was present in all dogs dosed with 20 mg/kg/day Formulation XII. Test item-related, mild carinal and tracheal mucosal lymphocytic inflammation was present in 2 of 3 dogs dosed with 20 mg/kg/day Formulation XII. In additional, minimal, intra-alveolar, mixed cell inflammation was present in 1 of 3 dogs dosed with 20 mg/kg/day Formulation XV. Therefore, the location of findings varied somewhat between Formulation XII and Formulation XV. The variability complicates comparison of Formulation XII to Formulation XV, although the dose level at which clearly test item-related findings occurred (20 mg/kg/day) was the same for both test items. Mild mixed cell inflammation was present in 1 of 3 dogs dosed with 6 mg/kg Formulation XII every three days and 1 of 3 dogs dosed with 6 mg/kg/day Formulation XV. Due to the low incidence of this finding in each of these groups and the lack of findings in other areas of the respiratory tract at these doses, the relationship of inhalation of Formulation XII or Formulation XV was unclear.

### Comparison of Formulation XIX to Formulation XII and Formulation XV in Rat Studies

Formulation XIX-related findings in the respiratory tract of rats had a different character from those induced by Formulation XII and Formulation XV. In addition, Formulation XIX-related findings involved more regions (tissues) in the respiratory tract and likely were adverse at a lower dose. Recovery was not evaluated in the rat studies with Formulation XII or Formulation XV. However, based on experience with other inhaled materials, it is likely that granulomatous inflammation within the bronchiolar mucosa, which was present after exposure to Formulation XIX, would resolve more slowly than increased alveolar macrophages or subacute inflammation included by Formulation XII or Formulation XV.

Granulomas or granulomatous inflammation composed of macrophages and multinucleated giant cells are common responses to materials that are not readily solubilized within cytoplasmic lysosomes, including aspirated foreign bodies. The presence of these cells in the mucosa at multiple levels in the respiratory tract after inhalation of Formulation XIX suggests that test item impacted and either a) penetrated the epithelium and was phagocytosed by macrophages, or b) dissolved, penetrated the epithelium, and recrystallized, in the interstitium where it was phagocytosed by macrophages, or b) dissolved, penetrated the epithelium, and recrystallized, in the interstitium where it was phagocytosed by macrophages. The lack of complete resolution during the recovery period is not unexpected for a material in a poorly soluble form.

Formulation XII dosed at 40 mg/kg/day exposure resulted in mild subacute inflammation, which was considered test item related and adverse. This subacute inflammation occurred in the alveolar parenchyma and was morphologically different from the granulomatous mucosal inflammation that occurred with Formulation XIX exposure. It was not clear whether minimal subacute inflammation, which occurred in rats dosed at 15 m/kg/day with Formulation XII or Formulation XV, was test item related. In comparing Formulation XII to Formulation XV, there was no clear difference in the incidence and severity of macrophage accumulation or subacute inflammation among male rats dosed with these test items at comparable dose levels. There was a suggestion of a higher severity and/or incidence of these findings in female rats dosed at 15 mg/kg/day with Formulation XV compared with Formulation XII. Recovery was not investigated in the studies with Formulation XII or Formulation XV. However, minimal to mild subacute inflammation and minimal to mild macrophage accumulation are considered reversible findings and would generally be expected to resolve in a 28-day recovery period.

It is difficult to compare alveolar macrophage accumulation across these rat studies because the Formulation XII or Formulation XV study did not include air or placebo controls. Different groups of rats at different testing facilities can have different background incidences of minimal alveolar macrophage accumulation. However, the data from these studies suggest that minimal to mild alveolar macrophage accumulation may have occurred at higher incidences and/or lower doses in rats dosed with Formulation XII or Formulation XV, perhaps indicating greater dispersal in the alveoli of Formulation XII or Formulation XV in a form that was phagocytosed by macrophages.

### Comparison of Formulation XIX to Formulation XII and Formulation XV in Dog Studies

Formulation XIX-related findings in the respiratory tract of dogs had a different character from those induced by Formulation XII and Formulation XV.

Formulation XIX-related acute inflammation, which was considered adverse, was present in the 7-day dog study at 10 mg/kg/day. There were no Formulation XIX-related findings at 5 mg/kg/day in the 7-day study. Formulation XIX-related granulomatous inflammation was present at ≥5 mg/kg/day in the 28-day dog study and it reached a mild severity where it was considered adverse at ≥10 mg/kg/day. Retrospectively, the acute inflammation observed in the 7-day study could be described as acute, granulomatous inflammation. Thus, the findings were similar across the two dog studies with Formulation XIX, but with differences reflecting the length of the studies. In both studies, the findings were considered adverse at 10 mg/kg/day. The location of the inflammation was primarily bronchiolar/peribronchiolar, as opposed to within alveoli. This location indicates a conducting airway orientation and thus it is somewhat similar to the location of the Formulation XIX-related finding in rats, although it did not exhibit the mucosal location and was not as discreet as the rat finding. Granulomatous inflammation was similar, but not morphologically identical in the rat and dog.

Formulation XII or Formulation XV induced test item-related findings at 20 mg/kg/day in the 14-day dog study. Test item-related, mild, intra-alveolar, mixed cell inflammation was present in all dogs dosed with 20 mg/kg/day Formulation XII. Adversity was not addressed in the 14-day study report, but mild mixed cell inflammation would likely be considered adverse. Findings at 6 mg/kg/day were not clearly test item related due to the low incidence. Dogs were not exposed to Formulation XII or Formulation XV at 10 mg/kg/day, so a direct comparison to Formulation XIX, which was adverse at 10 mg/kg/day cannot be made. However, it might be more appropriate to compare lung tissue levels as opposed to doses when comparing adversity after inhalation exposure and these were substantially higher in animals dosed with Formulation XII and Formulation XV formulations. Granulomatous inflammation associated with Formulation XIX completely resolved during the 28-day recovery period. The 14-day dog study with Formulation XII and Formulation XV did not include a recovery period. Formulation XII or Formulation XV-related mixed cell inflammation in the dog was morphologically somewhat similar to Formulation XII or Formulation XV-related subacute inflammation in the rat in that it involved the alveoli and was not granulomatous.

### Example 21; Phase 1 Open-label Study to Assess Safety, Tolerability and Pharmacokinetics of Single and Multiple Doses of Itraconazole Administered as a Dry Powder for Inhalation in Healthy Subjects

### Clinical Pharmacokinetics of Itraconazole Oral Solution Based on Historical Data

Itraconazole is metabolized in liver by the cytochrome P450 3A4 isoenzyme system to the major metabolize hydroxy-itraconazole. Itraconazole is highly bound by plasma protein. 99.8% and 99.6%, oral solution and capsules, respectively.

The pharmacokinetics of oral itraconazole have been studied in both single and multiple dose studies in humans. The pharmacokinetics differ between the two presentations (solution and capsules), with higher exposure observed with the oral solution. It is recommended that the oral solution and capsules not be used interchangeably.

The absolute bioavailability of the oral solution is 55% in healthy volunteers, and increases by 30% when taken under fasted conditions. Under fasted conditions, the steady-state AUC₀₋₂₄ₕ is 131±30% of the exposure under fed conditions and it is recommended that the oral solution be administered fasted. At steady-state, under fasted conditions the mean Cₘₐₓ, tₘₐₓ and AUC₀₋₂₄ₕ of a 200 mg daily dose of itraconazole was 1,963±601 ng/ml, 2.5±0.8 h and 29,271±10,285 ng·h/mL, respectively. The half-life of itraconazole at steady state was 39.7±13 h.

### Pharmacokinetic Data from Part 1. SAD in Healthy Volunteers

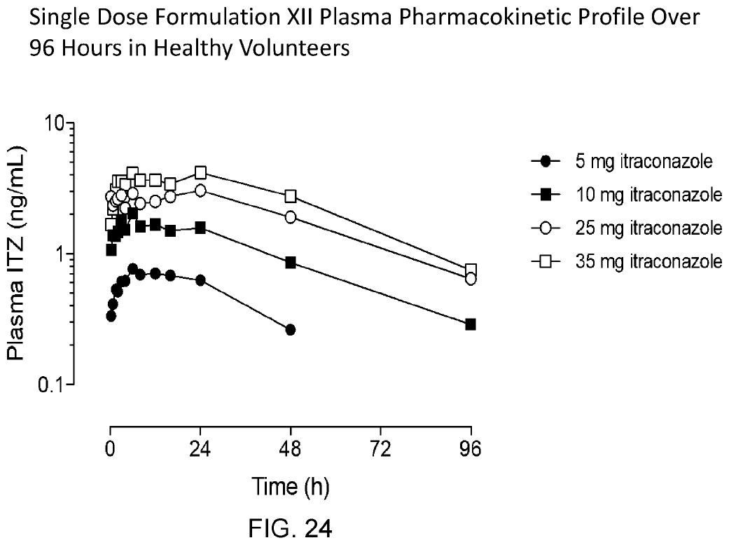

Summary data for systemic pharmacokinetics after a single inhaled dose of Formulation XII are summarized in Table 69 and concentration-time profiles are shown in FIG. 24. The pharmacokinetic data is important because of the effect it has on the safety profile of Formulation XII compared to oral dosing. The data confirms that inhaled dosing with Formulation XII results in low systemic exposure. Doses ranged from 5 mg to 35 mg of itraconazole. Itraconazole was rapidly absorbed into the systemic circulation, with all subjects having detectable plasma exposure at the earliest sampling timepoint of 15 minutes. Exposure was generally maintained during the first 18-24 hours indicating a prolonged absorption. Beyond 24 hours after dosing, plasma concentrations generally declined in a steady mono-exponential manner with the rate of decay similar across all cohorts (range Kel geometric means; 0.021-0.032 1/h). Exposure (Cₘₐₓ and AUC) increased monotonically and were generally less than dose-proportional.

TABLE 69
Pharmacokinetic data following single, inhaled
doses of Formulation XII in healthy volunteers

| Nominal | | | | | |
|---|---|---|---|---|---|
| Itraconazole | | | | | |
| Dose | AUC_{0-24 h} | AUC₀₋ₗₐₛₜ | AUC_{0-inf} | Cₘₐₓ | tₘₐₓ* |
| (mg) | (h · ng/mL) | (h · ng/mL) | (h · ng/mL) | (ng/mL) | (h) |
| 5 | 15.9 | 26.1 | 30.2^{a} | 0.87 | 6 |
| 10 | 38.8 | 93.5 | 110.4^{b} | 2.28 | 6 |
| 25 | 64.64 | 179.4 | 211.3 | 3.9 | 3 |
| 35 | 86.76 | 224.5 | 182.7^{c} | 4.58 | 18 |

Data shown are the geometric mean values for each cohort (n = 5 for the 5 mg dose; n = 6 for the 10, 25 and 35 mg doses).
*median values.

### Pharmacokinetic Data from Part 2, MAD in Healthy Volunteers

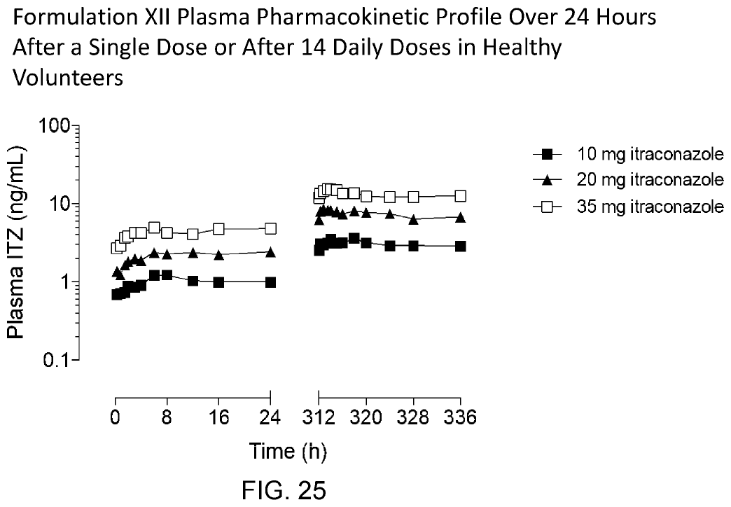

Summary data for systemic pharmacokinetics after a single inhaled dose and 14 days of daily inhalation of Formulation XII are summarized in Table 70 and concentration-time profiles are shown in FIG. 25. Doses were either 10 mg, 20 mg or 35 mg of itraconazole. As in Part 1, itraconazole was rapidly absorbed into the systemic circulation with all subjects having detectable plasma exposure at the earliest sampling timepoint of 15 minutes. Exposure was generally maintained during the first 18-24 hours with median Tₘₐₓ estimates between 7 hours and 18 hours across cohorts.

Median plasma concentration increased with each repeat dose, with concentrations close to steady state by Day 14. Compared to steady-state plasma levels of itraconazole reported after dosing with the oral solution, exposure following inhalation was 100- to 400-fold lower based on AUC₀₋₂₄ₕ. Between Day 1 and Day 14 itraconazole accumulation was approximately 3-fold for both Cₘₐₓ and AUC₀₋₂₄ₕ and similar for each dose. As in Part 1, at the end of dosing, plasma concentrations declined in a steady mono-exponential manner suggesting the absence of any exaggerated lung accumulation that would result in a prolonged systemic exposure.

TABLE 70
Pharmacokinetic data following single and multiple, inhaled
doses of Formulation XII in healthy volunteers

| Nominal | DAY 1 | | | DAY 14 | | | | |
|---|---|---|---|---|---|---|---|---|
| Itraconazole | AUC_{0-24 h} | Cₘₐₓ | tₘₐₓ* | AUC_{0-24 h} | Cₘₐₓ | tₘₐₓ* | Accumulation | |
| Dose (mg) | (h · ng/mL) | (ng/mL) | (h) | (h · ng/mL) | (ng/mL) | (h) | AUC_{0-24 h} | Cₘₐₓ |
| 10 | 24.4 | 1.29 | 7 | 73.2 | 3.77 | 5 | 3.0 | 2.9 |
| 20 | 53.0 | 2.64 | 18 | 174.4 | 8.98 | 4 | 3.3 | 3.4 |
| 35 | 102.4 | 5.23 | 7 | 276.5 | 15.2 | 0.8 | 2.7 | 2.9 |

Data shown are the geometric mean values for each cohort (n = 6 for the 10 mg and 20 mg dose; n = 6 for day 1 and n = 5 for day 14 for the 35 mg dose).
*median values.

### Pharmacokinetic Data from Part 3, Single Doses in Adult Subjects with Mild-to-Moderate Stable Asthma

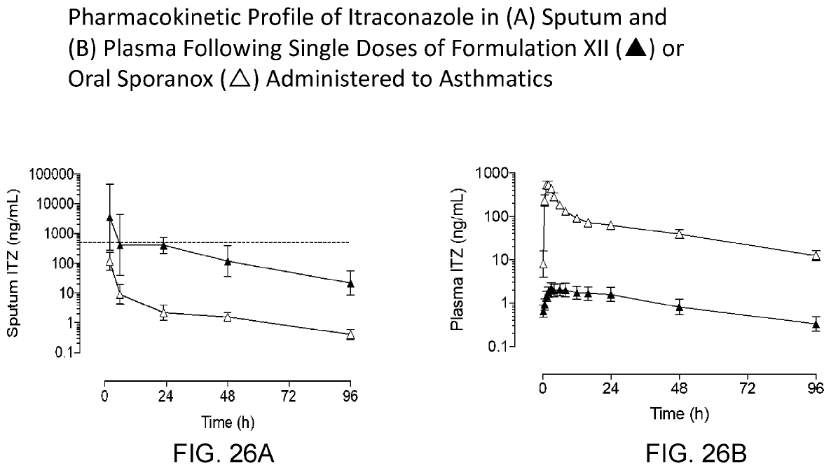

Summary data for systemic pharmacokinetics after a single inhaled or oral dose in asthma patients are summarized in Table 71 and concentration time-profiles are shown in FIG. 26A and FIG. 26B. Doses were either 20 mg itraconazole inhaled as Formulation XII or 200 mg of itraconazole administered as SPORANOX® oral solution. For both oral and inhaled doses, itraconazole was quickly absorbed into the systemic circulation with median Tₘₐₓ estimates of 4.0 hours and 1.5 hours for Formulation XII and SPORANOX® respectively. Following Formulation XII administration, itraconazole plasma exposure generally increased and/or was maintained over the first 24 hours indicating a prolonged absorption. In contrast, orally administered itraconazole was rapidly absorbed and eliminated, such that exposure peaked soon after dosing, but rapidly declined to levels that are 17% of Cₘₐₓ 12 hours after dosing. Total systemic exposure over 24 hours (AUC₀₋₂₄ₕ), was approximately 85-fold lower after Formulation XII relative to exposure after oral dosing and maximum exposure (Cₘₐₓ), was approximately 250-fold lower after Formulation XII relative to exposure after oral dosing.

TABLE 71
Pharmacokinetic data following single doses of Formulation
XII or oral SPORANOX ® in asthma patients

| | Nominal | | | | | |
|---|---|---|---|---|---|---|
| | Dose | AUC_{0-24 h} | AUC₀₋ₗₐₛₜ | AUC_{0-inf} | Cₘₐₓ | tₘₐₓ* |
| | (mg) | (h · ng/mL) | (h · ng/mL) | (h · ng/mL) | (ng/mL) | (h) |
| Formulation | 20 | 41.8 | 90.5 | 105.2 | 2.33 | 4 |
| XII | | | | | | |
| Sporanox | 200 | 3618 | 6118 | 6649 | 581 | 1.5 |

Data shown are the geometric mean values for each cohort (n = 14 for Formulation XII and n = 15 for SPORANOX ®).
*median values.

Induced sputum was collected 2 hours, 6 hours, and 24 hours after dosing and used to measure itraconazole concentrations using a validated liquid chromatography-mass spectrometry/mass spectrometry (LC-MS/MS) method with a LLOQ (lower limits of quantification) of 0.1 ng/mL. Sputum itraconazole levels were higher with Formulation XII dosing relative to oral SPORANOX® dosing, with a geometric mean Cₘₐₓ after inhalation of 5381 ng/mL compared to a Cₘₐₓ of 116.3 ng/mL after oral dosing (FIG. 26A). High lung exposure following Formulation XII was maintained over a 24 hour period, whereas sputum concentrations of itraconazole decreased between 2 hours and 6 hours after a single 200 mg oral itraconazole dose. These data confirm that inhaled dosing with Formulation XII results in high and sustained lung exposure, higher than what is achieved with oral dosing, while maintaining low systemic exposure. Based on geometric mean Cₘₐₓ data in lung and plasma, Formulation XII resulted in a lung:ratio of approximately 2300:1 and oral dosing resulted in a lung; plasma ratio of 1:5.

### Example 22. Dry Powder Formulations Comprising Itraconazole in Crystalline Particulate Form at Varying Drug Loads

The nanocrystalline itraconazole for Formulations XXXXI-XXXXVI was prepared as a suspension comprising 35.0 wt % itraconazole (SMS Pharma lot ITZ-0715005) and 2.92 wt % polysorbate 80, comprising a 12:1 ratio (wt:wt) of itraconazole to polysorbate 80. The polysorbate 80 was dissolved in 62.1% deionized water via magnetic stir bar, then the itraconazole was added and suspended by stirring with a magnetic stir bar. Once all of the itraconazole was suspended, the formulation was processed on the Netzsch MiniCer using 0.2 mm grinding media (TOSOH, Tokyo, Japan) with 90% chamber fill. The following conditions were used to manufacture the itraconazole suspension. The mill speed was 3000 RPM, the inlet pump flow rate was 220 mL/min, the recirculating chiller was 10° C., and the run time was 37 minutes. The final median particle size (Dv(50)) of the milled suspension was 141 nm.

Feedstock suspensions were prepared and used to manufacture dry powders comprising itraconazole in crystalline particulate form and additional excipients. Drug loads of 50, 60, 70 and 80 wt % itraconazole, on a dry basis, were targeted. The feedstock suspensions that were used to spray dry particles were made as follows. The required quantity of water was weighed into a suitably sized glass vessel. The excipients were added to the water and the solution was allowed to stir until visually clear. The itraconazole-containing nano-suspension was then added to the excipient solution and stirred until visually homogenous. The feedstocks were then spray-dried. Feedstocks were stirred while spray dried. Table 72 lists the components of the feedstocks used in preparation of the dry powders.

TABLE 72
Feedstock compositions for Formulations XXXXI-XXXXVI

| | | | | Sodium | | |
|---|---|---|---|---|---|---|
| | Water | Itraconazole | Polysorbate | sulfate | Leucine | Total mass |
| Formulation | (g) | (g) | 80 (g) | (g) | (g) | (g) |
| XXXXI | 67.891 | 1.050 | 0.087 | 0.749 | 0.216 | 69.992 |
| XXXXII | 67.918 | 1.260 | 0.105 | 0.573 | 0.162 | 70.019 |
| XXXXIII | 67.911 | 1.470 | 0.122 | 0.395 | 0.114 | 70.012 |
| XXXXIV | 67.910 | 1.680 | 0.140 | 0.218 | 0.062 | 70.010 |
| XXXXV | 67.896 | 1.680 | 0.140 | 0.139 | 0.141 | 69.996 |
| XXXXVI | 67.897 | 1.680 | 0.140 | 0.062 | 0.218 | 69.996 |

Dry powders of Formulations XXXXI-XXXXVI were manufactured from the corresponding feedstocks in Table 72 by spray drying on the Büchi B-290 Mini Spray Dryer (BÜCHI Labortechnik AG, Flawil, Switzerland) with cyclone powder collection. The system was run in open-loop (single pass) mode using nitrogen as the drying and atomization gas. Atomization of the liquid feed utilized a Schlick 970-1 nozzle. The aspirator of the system was adjusted to maintain the system pressure at −2.0″ water column.

The following spray drying conditions were followed to manufacture the dry powders. The liquid feedstock solids concentration was 30 g/kg, the drying gas flowrate was 17.0 kg/hr, the atomization gas flowrate was 19.6 g/min, and the liquid feedstock flowrate was 3.0 mL/min. The process gas inlet temperature was varied to keep the outlet temperature constant at 65° C. The resulting dry powder formulations are reported in Table 73.

TABLE 73
Formulation XXXXI-XXXXVI compositions, dry basis

| Formulation | Dry Powder Composition (w/w), dry basis |
|---|---|
| XXXXI | 50.0% itraconazole, 35.7% sodium sulfate, |
| | 10.2% leucine, 4.16% polysorbate 80 |
| XXXXII | 60.0% itraconazole, 27.2% sodium sulfate, |
| | 7.78% leucine, 5.00% polysorbate 80 |
| XXXXIII | 70.0% itraconazole, 18.8% sodium sulfate, |
| | 5.37% leucine, 5.83% polysorbate 80 |
| XXXXIV | 80.0% itraconazole, 10.4% sodium sulfate, |
| | 2.96% leucine, 6.67% polysorbate 80 |
| XXXXV | 80.0% itraconazole, 6.67% sodium sulfate, |
| | 6.66% leucine, 6.67% polysorbate 80 |
| XXXXVI | 80.0% itraconazole, 2.96% sodium sulfate, |
| | 10.4% leucine, 6.67% polysorbate 80 |

### B. Powder Characterization

The bulk particle size characteristics for the six formulations are found in Table 74. The 1 bar/4 bar dispersibility ratio less than 1.1 and 0.5 bar/4 bar dispersibility ratio less than 1.25 for Formulations XXI-XXXXVI indicate that they are relatively independent of dispersion energy, a desirable characteristic which allows similar particle dispersion across a range of dispersion energies.

TABLE 74
Formulation XXXXI-XXXXVI Bulk particle size

| | 1 bar | 1 bar:4 bar | 0.5 bar:4 bar |
|---|---|---|---|
| | Dv[50] | Dv[50] | Dv[50] |
| Formulation | (μm) | ratio | ratio |
| XXXXI | 2.47 | 1.01 | 1.11 |
| XXXXII | 2.73 | 1.08 | 1.15 |
| XXXXIII | 2.65 | 1.08 | 1.20 |
| XXXXIV | 2.61 | 1.04 | 1.14 |
| XXXXV | 2.85 | 1.00 | 1.00 |
| XXXXVI | 2.40 | 1.02 | 1.15 |

The weight loss of Formulations XXXXI-XXXXVI was measured via TGA and is detailed in Table 7.

TABLE 75
Formulation XXXXI-XXXXVI weight loss via TGA

| Formulation | Weight loss via TGA (%) |
|---|---|
| XXXXI | 0.23 |
| XXXXII | 0.69 |
| XXXXIII | 0.18 |
| XXXXIV | 0.33 |
| XXXXV | 0.64 |
| XXXXVI | 0.37 |

The aerodynamic particle size, fine particle fractions, and fine particle doses measured and/or calculated with a Next Generation Impactor (NGI) for Formulations XXXXI, XXXXIV and XXXXVI are reported in Table 76. The fine particle doses for all formulations indicate a high percentage of the nominal dose which is filled into the capsule reaches the impactor stages (>45%) and so would be predicted to be delivered to the lungs. The MMADs of all formulations were <3.5 μm microns, indicating deposition in the central and conducting airways.

TABLE 76
Formulation XXXXI-XXXXVI aPSD via NGI

| | MMAD | FPD <5 μm |
|---|---|---|
| Formulation | (μm) | (% nominal dose) |
| XXXXI | 3.12 | 53.6 |
| XXXXIV | 3.18 | 48.8 |
| XXXXVI | 3.31 | 54.8 |

1. A method of treating cancer, comprising administering to the respiratory tract of a subject in need thereof a dry powder, wherein the dry powder comprises homogenous respirable dry particles that comprise itraconazole and one or more excipients.

2. The method of claim 1, wherein the cancer is lung cancer.

3. The method of claim 2, wherein the lung cancer is non-small cell lung cancer (NSCLC).

4-6. (canceled)

7. The method of claim 1, wherein the subject is receiving an additional therapeutic agent.

8. The method of claim 7, wherein the additional therapeutic agent is a chemotherapeutic drug, an immunotherapy drug, or a targeted cancer therapy drug.

9-14. (canceled)

15. The method of claim 1, wherein the dry powder is administered to the subject before surgery to remove cancerous tissue from the subject.

16. The method of claim 1, wherein the dry powder is administered to the subject before, concurrently with, or after performing radiation therapy.

17. The method of claim 1, wherein the itraconazole is crystalline.

18. The method of claim 1, wherein the dry powder further comprises a stabilizer.

19-21. (canceled)

22. The method of claim 1, wherein the itraconazole is present in the respirable dry particles as a crystalline sub-particle.

23. The method of claim 22, wherein the sub-particle has a Dv50 of about 50 nm to about 5,000 nm.

24-25. (canceled)

26. The method of claim 1, wherein the ratio of itraconazole:stabilizer (wt:wt) in the respirable dry particles is greater than or equal to 10:1.

27. The method of claim 18, wherein the stabilizer is present in the respirable dry particles in an amount of about 4% to about 10% by weight.

28-30. (canceled)

31. The method of claim 1, wherein the one or more excipients comprise a sodium salt and an amino acid.

32. The method of claim 31, wherein the sodium salt is selected from the group consisting of sodium chloride and sodium sulfate, and the amino acid is leucine.

33. The method of claim 18, wherein the stabilizer is polysorbate 80.

34. (canceled)

35. The method of claim 1, wherein the respirable dry particles have;
(i) a volume median geometric diameter (VMGD) of about 10 microns or less;
(ii) a tap density of between 0.2 g/cc and 1.0 g/cc;
(iii) a 1 bar/4 bar dispersibility ratio (1/4 bar) of less than about 1.5, as measured by laser diffraction; and/or
(iv) a 0.5 bar/4 bar dispersibility ratio (0.5/4 bar) of about 1.5 or less, as measured by laser diffraction.

36. The method of claim 1, wherein the dry powder has;
(i) a mass median aerodynamic diameter (MMAD) of between about 1 micron and about 5 microns; and/or
(ii) a fine particle fraction (FPF) of the total dose less than 5 microns of about 25% or more.

37. The method of claim 1, wherein the respirable dry particles have a capsule emitted powder mass of at least 80% when emitted from a passive dry powder inhaler that has a resistance of about 0.036 sqrt(kPa)/liters per minute under the following conditions; an inhalation flow rate of 30 LPM for a period of 3 seconds using a size 3 capsule that contains a total mass of 10 mg, said total mass consisting of the respirable dry particles, and wherein the volume median geometric diameter of the respirable dry particles emitted from the inhaler as measured by laser diffraction is 5 microns or less.

38. (canceled)

39. The method of claim 1, wherein the dry powder is administered in an amount effective to achieve:
a) a lung concentration of itraconazole in sputum of at least about 100 ng/mL;
b) a lung concentration of itraconazole in tissue of at least about 100 ng/g; or
c) a plasma concentration of itraconazole of no more than 25 ng/mL.

40-43. (canceled)
